(19) United States
(12) Patent Application Publication
(10) Pub. No.: US 2025/0127906 A1
(43) Pub. Date: Apr. 24, 2025

(54) SMALL-MOLECULE DEGRADERS OF CDK8 AND CDK19

(71) Applicants: University of South Carolina, Columbia, SC (US); Senex Biotechnology, Inc., Columbia, SC (US)

(72) Inventors: Igor RONINSON, Lexington, SC (US); Mengqian CHEN, Lexington, SC (US); Li ZHANG, Columbia, SC (US); Campbell MCINNES, Irmo, SC (US)

(21) Appl. No.: 18/707,847

(22) PCT Filed: Nov. 7, 2022
(86) PCT No.: PCT/US2022/049109

§ 371 Date: May 6, 2024

Related U.S. Application Data
(60) Provisional application No. 63/276,356, filed on Nov. 5, 2021.

(51) Int. Cl. A61K 47/55; A61P 35/00; A61P 35/02

(52) U.S. Cl. CPC A61K 47/55; A61P 35/00; A61P 35/02

(57) ABSTRACT

Disclosed herein are CDK8/19 degraders and methods of making and using the same. The CDK8/19 degrader is a heterobifunctional compound, or a pharmaceutically acceptable salt thereof, comprising a CDK8/19 targeting moiety, a ubiquitin ligase ligand moiety, and a linker linking the CDK8/19 targeting moiety and the ubiquitin ligase ligand moiety.

### CROSS-REFERENCE TO RELATED APPLICATIONS

This application claims priority to U.S. Provisional Application No. 63/276,356 filed on Nov. 5, 2021, the contents of which are incorporated by reference in its entirety.

### STATEMENT REGARDING FEDERALLY SPONSORED RESEARCH

This invention was made with government support under P20GM109091 and R44CA203184 awarded by the National Institutes of Health. The government has certain rights in the invention.

### BACKGROUND OF THE INVENTION

CDK8 and CDK19, two closely related transcription-regulating kinases, have become a burgeoning novel cancer drug target (Philip et al., 2018). In particular, CDK8/19 inhibitors were shown to be efficacious in castration-refractory prostate cancer (CRPC) (Chen, Roninson, U.S. Pat. No. 9,636,342), in acute myeloid leukemia (Pelish et al., 2015), in hepatic metastases of colon cancer (Liang et al., 2018), in estrogen receptor-positive breast cancer (McDermott et al., 2017) and in HER2-positive breast cancer (McDermott et al., International Patent Pub. No. WO 2016/018511). Many other cancers were suggested by informatic analysis to be potentially dependent on CDK8/19 (Roninson et al., 2019). Furthermore, CDK8/19 inhibitors prevent the induction of genes that promote metastasis and drug resistance in cancer cells of different tumor types, treated with conventional DNA-damaging chemotherapeutic agents or radiation (Porter et al., 2012), indicating the utility of CDK8/19 inhibitors for the treatment of different cancers when combined with a variety of DNA-damaging agents.

Aside from cancer, CDK8/19 inhibitors show promise in inflammation-associated diseases (US Patent Pub. No. 2014/0309224 to Porter, D.C.) (Johannessen et al., 2017); cardiovascular diseases (Walz et al., 2017) (International Patent Pub. No. WO 2016/100782 to Roninson, I. B.); ribosomopathies; conditions characterized by reduced number of hematopoietic stem cells and/or progenitor cells; and bone anabolic disorders (International Patent Pub. No. WO 2017/076968 to Flygare, A.) (Amirhosseini et al., 2019) and viral diseases (US Patent Pub. No. 2014/0309224 to Porter, D.C.) (Butler et al., 2020). CDK8/19 are required for embryonic development, a process driven by transcriptional reprogramming (Lynch et al., 2020; Westerling et al., 2007), but CDK8 knockout has no phenotypic effects in adult animals (Adler et al., 2012).

Selective CDK8/19 inhibitors have been disclosed, including those by some of the instant inventors (e.g., U.S. Pat. No. 8,598,344B2, U.S. Pat. No. 9,321,737B2, U.S. Pat. No. 9,409,873B2, U.S. Pat. No. 11,014,906; WO2020160537; and WO2020237014) and by others (reviewed in (Ma et al., 2020; Philip et al., 2018, Xi et al., 2019)); see also (Al-Sanea, 2020; Grandjean et al., 2020; Hofmann et al., 2020; Li et al., 2020; Martinez-González et al., 2020; Solum et al., 2020; Yu et al., 2021a; Yu et al., 2021b). Although systemic toxicity was reported for two Mediator kinase inhibitors (Clarke et al., 2016), this toxicity was later shown to be due to off-target effects of these compounds (Chen et al., 2019).

Heterobifunctional molecules, PROteolysis TArgeting Chimeras (PROTACs) (reviewed in (Samarasinghe and Crews, 2021)) are known to degrade proteins. CDK8-targeting PROTACs, based on a small-molecule kinase inhibitor JH-VIII-49, have been reported (Hatcher et al., 2018) (US 2021/0054020 to Nathanael S. Gray). The resulting compound JH-XI-10-02 (a.k.a. 29) strongly induced CDK8 protein degradation in Jurkat leukemia cell line after 24 hrs treatment at 1 μM and partially degraded CDK8 at 100 nM. However, JH-XI-10-02 induced no CDK19 degradation in Jurkat cells. In contrast to Jurkat cells, JH-XI-10-02 produced only a moderate decrease in CDK8 protein even at 5 μM in another leukemia cell line, Molt4.

There is a need to develop CDK8 PROTACs that are more potent that JH-XI-10-02 in inducing CDK8 degradation in different cell lines. It is also desirable to have PROTACs decrease the protein levels of CDK19, which can substitute for CDK8 in many biological functions. Such CDK8/19 degraders would provide valuable tools to distinguish between the kinase-dependent and kinase-independent activities of CDK8 and CDK19 and may also have a therapeutic value in suppressing kinase-independent disease-promoting activities of these Mediator kinases.

### BRIEF SUMMARY OF THE INVENTION

Disclosed herein are CDK8/19 degraders and methods of making and using the same. The CDK8/19 degrader is a heterobifunctional compound, or a pharmaceutically acceptable salt thereof, comprising a CDK8/19 targeting moiety, a ubiquitin ligase ligand moiety, and a linker linking the CDK8/19 targeting moiety and the ubiquitin ligase ligand moiety. The CDK8/19 targeting moiety may comprise

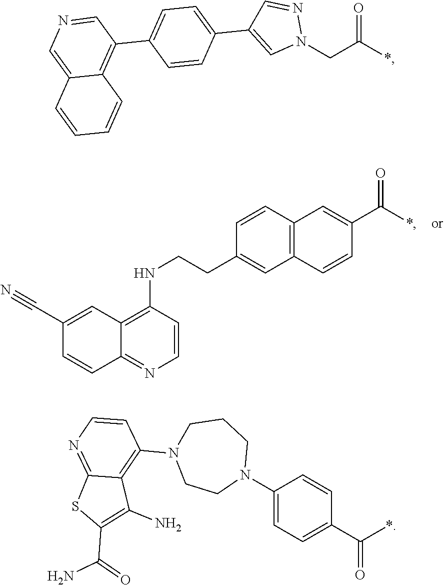

The * indicates a point of attachment of the CDK8/19 targeting moiety for the compound. In some embodiments, wherein the CDK8/19 ligand comprises a piperazine between the CDK8/19 targeting moiety and the linker. In some embodiments, the ubiquitin ligase ligand moiety comprises a pomalidomide moiety. In some embodiments, the pomalidomide ubiquitin ligase ligand moiety additionally comprises a methyl pivalate group.

Another aspect of the invention provides for a method for treating a subject. The method comprises administering to the subject an effective amount of the compounds described herein. In some embodiments, the subject is in need of a treatment for a cancer, such as a leukemia, blood, prostate, breast, colon, nervous system, or osteo cancer. In some embodiments, the subject is in need of an agent to reduce drug resistance to an anticancer agent. In some embodiments, the subject has a viral disease. In some embodiments, the subject has an inflammation-associated disease. In some embodiments, the subject has a ribosomopathy. In some embodiments, the subject has a condition characterized by reduced number of hematopoietic stem cells and/or progenitor cells. In some embodiments, the subject has a bone anabolic disorder.

Another aspect of the invention provides for a method for the degradation of CDK8 or CDK19. The method comprises contacting CDK8 or CDK19 with any of the compounds described herein in a proteolytic environment. In some embodiments, the method degrades both CDK8 and CDK19.

Another aspect of the invention provides for a method for the degradation of cyclin C. The method comprises contacting CDK8 or CDK19 with any of the compounds described herein in a proteolytic environment having cyclin C therein.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Chemical structure of representative PROTACs.

FIG. 2. Effects of 24-hr treatment with different PROTAC compounds on CDK8/CDK19/CCNC protein levels in HEK-293cells.

FIG. 3. Effects of co-treatment with MG132, Pomalidomide and Senexin C on HP8580-mediated CDK8 degradation in HEK-293cells.

FIG. 4. Effects of 24-hr treatment with different PROTAC compounds on CDK8/CDK19/CCNC protein levels in Jurkat cells.

FIG. 5. Effects of 24-hr treatment with different PROTAC compounds on CDK8/CDK19 protein levels in different human cell lines (A: 22Rv1; B: MV4-11; C: SJSA-1, HTB-185 and HAP-1; D: SAOS2).

FIG. 6. Effects of treatment with different PROTAC compounds on CDK8/CDK19 protein levels in non-human cell lines (A: murine CT26; B: canine Abrams).

FIG. 7. Time course of CDK8/CDK19/CCNC degradation by IA7886 in HEK-293 and CT26 cells.

FIG. 8. Effects of 24-hr treatment with different CDK8/19i and PROTACs on STAT1-S727 phosphorylation and CDK8 degradation.

FIG. 9. Effects of 3-day treatment with 200 nM BI-1347 and 200 nM IA7886 on CDK8/19-dependent gene expression in 293 parental (WT) and CDK8/19 double-knockout (dKO) cells.

FIG. 10. (A) Stability of transcriptional changes induced by BI-1347 (CDK8/19 kinase inhibitor) and IA7886 (CDK8/19 PROTAC) in the wash-off study in HEK-293cells. (B) Stability of pSTAT-S727 inhibition and CDK8/CDK19/CCNC degradation by IA7886 in 293 wash-off study.

FIG. 11. Long-term effects of CDK8/19 kinase inhibitor BI-1347 and CDK8/19 PROTAC IA7886 on growth of leukemia cells. (A) Jurkat cells were continuously treated with 0.1% DMSO, 300 nM BI-1347 or 300 nM IA7886 for 53 days. (B) MV4-11 cells were continuously treated with 300 nM BI-1347 or IA 7886 for 60 days. (C) Jurkat cells were pre-treated with 200 nM BI-1347 or IA7886 for 8 days before removal of drug-containing media and further cultured in media containing the same compound (at 200 nM) or in drug-free media for 8 days.

FIG. 12. DepMap analysis of the effects of CRISPR knockout (A) or RNAi knockdown (B) of CCNC on the proliferation of all cancer cell lines in DepMap database or multiple myeloma (MM) cell lines. MM cell lines used for experimental assays are indicated with arrows.

FIG. 13. Effects of CDK8/19 inhibitors and PROTACs on KMS-12-BM MM cells. (A) Effects of the indicated compounds on cell growth over 20 days. (B) Effects of different concentrations of BI-1347 CDK8/19 kinase inhibitor or IA7886 PROTAC on cell number after 7-day treatment. (C) Western blot analysis of the effects of indicated compounds at the same concentrations as in (A) on CDK8 levels (GAPDH: normalization control).

FIG. 14. Effects of CDK8/19 inhibitors and PROTACs on RPMI 8226 MM cells. (A) Effects of the indicated compounds on cell growth over 20 days. (B) Effects of different concentration of SNX631-6 CDK8/19 kinase inhibitor or IA7886 PROTAC on cell number after 7-day treatment. (C) Western blot analysis of the effects of indicated compounds at the same concentrations as in (A) on CDK8 levels (GAPDH: normalization control).

FIG. 15. Effects of the indicated CDK8/19 inhibitors and PROTACs on SK-MM-2 cell growth over 20 days.

FIG. 16. Levels of CDK8 protein and STAT1 S727 phosphorylation (A) and mRNA expression of the CDK8/19 dependent gene CCL12 (B) in CT26 tumors from Balb/c mice dosed orally with vehicle, 20 mg/kg IA7882 or 20 mg/kg IA7886. (C) Serum drug concentrations in female CD-1 mice administered with 15 mg/kg IA7882 or IA7886 via intraperitoneal injection.

FIG. 17. Effects of 24 hr treatment with different PROTACs (JH-XI-10-02, IA7843, IA7886) and CDK8/19 kinase inhibitors (Senexin C and BI-1347) in CCNC^{low} Loucy cells, Jurkat cells, HEK-293 parental (WT) or HEK-293-CCNC-knockout (CCNC—KO) cells.

### DETAILED DESCRIPTION OF THE INVENTION

Disclosed herein are CDK8/19 degraders and methods of making and using the same. CDK8 (ubiquitously expressed) and CDK19 (expressed in some cell types) (Tsutsui et al., 2011) are two isoforms of Mediator kinase, the enzymatic component of the CDK module that binds to the transcriptional Mediator protein complex. In addition to CDK8 or CDK19, the CDK module includes Cyclin C, MED12 and MED13 (Fant and Taatjes, 2019; Philip et al., 2018). Unlike better-known CDKs (such as CDK1, CDK2 or CDK4/6), CDK8/19 regulate transcription but not cell cycle progression. In contrast to other transcriptional CDKs, such as CDK7 or CDK9, Mediator kinase is not a part of the overall transcription machinery (Fant and Taatjes, 2019) but acts as a cofactor or modifier of several cancer-relevant transcription factors, including β-catenin/TCF/LEF (Firestein et al., 2008), SMADs (Alarcon et al.; Serrao et al.), Notch (Fryer et al., 2004), STATs (Bancerek et al., 2013), H1F1α (Galbraith et al., 2013), ER (McDermott et al., 2017), NFκB (Chen et al., 2017) and MYC (Adler et al., 2012; Andrysik et al., 2021; EV et al., 2015; Fukasawa et al., 2021). CDK8/19 Mediator kinase directly phosphorylates some transcription factors (SMADs, STATs, Notch) and in other cases acts through C-terminal phosphorylation of RNA polymerase II (Pol II), enabling the elongation of transcription. Importantly, CDK8/19 affect Pol II phosphorylation not globally but only in the specific context of newly induced genes (Chen et al., 2017; Donner et al., 2010; Galbraith et al., 2013), impacting primarily de novo-induced but not basal transcription (Chen et al., 2017; McDermott et al., 2017). Based on this unique activity, CDK8/19 were identified as regulators of transcriptional reprogramming (Chen et al., 2017; Fant and Taatjes, 2019; Steinparzer et al., 2019).

Some of the therapeutically relevant Mediator kinase activities, including effects on tumor cell proliferation, have been attributed to kinase-independent functions of CDK8 and CDK19. Kinase-independent effects have been reported for both CDK8 (Kapoor et al., 2010, Menzl et al, 2019) and CDK19 (Audetat et al., 2017; Steinparzer et al., 2019). Such kinase-independent activities would be insensitive to conventional kinase inhibitors but they could be overcome through CDK8/19 protein degradation.

PROTACs are heterobifunctional molecules consisting of a warhead that binds a protein of interest (POI), a linker, and a ligand that recruits an E3 ubiquitin ligase. By simultaneously binding to POI and E3 ligase, PROTACs can bring two proteins into proximity and facilitate ubiquitination for sequential proteasomal degradation of POI. As used herein, a proteolytic environment is a chemical environment having the necessary enzymes, co-factors, chemicals, substances, and conditions such that the proteolysis of a POI, such CDK8, CDK19, and cyclin C, is possible.

In contrast to the protein inhibition, the event-driven pharmacological mechanism of PROTACs is catalytic in nature. Lower than effective inhibition drug concentration would give sufficient and prolonged target degradation and avoid off-target toxicity caused by high-dose drugs.

### CDK8/19 Degraders

CDK8/19 degraders are heterobifunctional compounds that are capable of degrading one or both of CDK8 and CDK19 in a proteolytic environment. CDK8/19 degrader is a PROTAC that comprises three key regions: a CDK8/19 targeting moiety capable of binding to CDK8 and/or CDK19, a linker to link the CDK8/19 ligand to an E3 ligase ligand moiety, and the E3 ligase ligand moiety. The present technology utilizes a PROTAC strategy to degrade CDK8 and/or CDK19. Degrading the CDK8/19 impairs catalytic as well as non-catalytic proprieties of the protein. Degrading the CDK8 or CDK19 may be used in a therapeutic method to treat diseases, conditions, or disorders that depend on CDK8 or CDK19.

The CDK8/19 targeting moiety may comprise

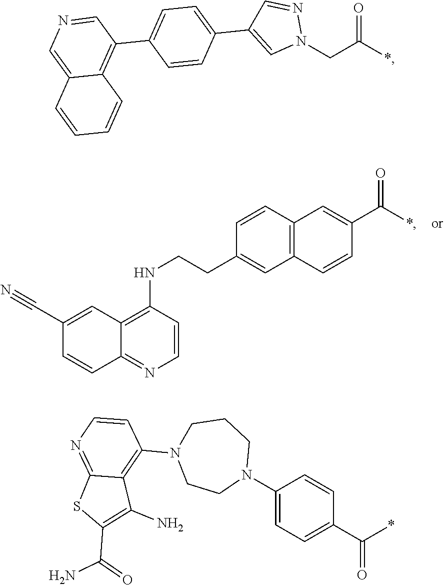

linked to the E3 ligase ligand moiety, wherein the * indicates a point of attachment for the CDK8/19 targeting moiety. As demonstrated in the Examples, the CDK8/19 degraders described herein are capable of degrading one or both of CDK8 and CDK19 in a proteolytic environment. Moreover, the CDK8/19 degraders are capable of degrading cyclin C in a proteolytic environment.

The compounds described herein recruit an E3 ligase. Suitably, the E3 ligase may include, without limitation, von Hippel-Lindau protein (VHL), CRBN, MDM2, cIAP, SKP2, and SPOP. In some embodiments, the E3 ligase is CRBN. In some embodiments, the ubiquitin ligase ligand moiety is a pomalidomide, thalidomide, lenalidomide, avadomide, or iberdomide group.

The protein of interest (POI) binding ligand and the ubiquitin ligase ligand may be chemically linked or coupled via a chemical linker (L). The linker should allow for appropriate formation of a target protein-ligase ternary complex. The linker group may comprise one or more structural units A, such as ethylene glycol (—CH₂CH₂O—) or alkylene (—CH₂) units.

In some embodiments, the linker may be a polyethylene glycol chain which may terminate (at either or both termini) in at least one of —P(O)(OH)O—, —S—, —N(R′)—, —C(O)—, —C(O)O—, —OC(O)—, OC(O)O—, —C(NOR)—, —C(O)N(R′)—, —C(O)N(R)C(O)—, —C(O)N(R)C(O)N(R′)—, —N(R)C(O)—, —N(R′)C(O)N(R)—, —N(R)C(O)O—, —OC(O)N(R)—, —C(NR′)—, —N(R)C(NR′)—, —C(NR′)N(R)—, —N(R)C(NR′)N(R)—, —S(O)₂—, —OS(O)—, —S(O)O—, —S(O)—, —OS(O)₂—, —S(O)₂O—, —N(R)S(O)₂—, —S(O)₂N(R)—, —N(R′)S(O)—, —S(O)N(R)—, —N(R)S(O)N(R′)—, —N(R′) S(O)N(R′)—, —S—S—, —O—Si(R)(R′)—O—, —C(═N)N(R)(R′)—, —C(R)═C(R′)—, —C≡C—, C3-12 carbocyclene, 3- to 12-membered heterocyclene, 5- to 12-membered heteroarylene or any combination thereof, wherein R is H, D, or C1-C50 alkyl, wherein the one or both terminating groups may be the same or different. In some embodiments, a polyethylene glycol chain may comprise 1-10 ethylene glycol units, 1-9 ethylene glycol units, 1-8 ethylene glycol units, 1-7 ethylene glycol units, 1-6 ethylene glycol units, 1-5 ethylene glycol units, 1-4 ethylene glycol units, 1-3 ethylene glycol units, 1-2 ethylene glycol units, or 1 ethylene glycol unit.

In some embodiments, the linker may be an alkylene chain or a bivalent alkylene chain, either of which may be interrupted by, and/or terminate (at either or both termini) in at least one of —P(O)(OH)O—, —O—, —S—, —N(R′)—, —C(O)—, —C(O)O—, —OC(O)—, —OC(O)O—, —C(NOR′)—, C (O)N(R′)—, —C(O)N(R′) C (O)—, —C(O)N(R)C(O)N(R′)—, —N(R)C(O)—, —N(R)C(O)N(R)—, —N(R)C(O)O)—, —OC(O)N(R)—, —C(NR)—, —N(R′)C(NR′)—, —C(NR′)N(R)—, —N(R′)C(NR)N(R′)—, —S(O)₂— —OS(O)—, —S(O)O—S(O)—, —OS(O)₂—, —S(O)₂O—, —N(R)S(O)₂—, —S(O)₂N(R)—, —N(R′) S(O)—, —S(O)N(R′)—, —N(R)S(O)₂N(R′)—, —N(R)S(O)N(R)—, —S—S—, —O—Si(R)(R′)—O—,)—, —C(═N)N(R)(R′)—, —C(R)═C(R′)—, —C≡C—, C1-C12 carbocyclene, 3- to 12-membered heterocyclene, 5- to 12-membered heteroarylene or any combination thereof, wherein R is H or C1-C50 alkyl, wherein the interrupting and the one or both terminating groups may be the same or different. In some embodiments, the alkylene chain may comprise 1-30 alkylene units, 1-25 alkylene units, 1-20 alkylene units, 1-15 alkylene units, 1-10 alkylene units, or 1-5 alkylene units.

In some embodiments, a group is positioned between the CDK8/19 targeting moiety and a linker described above. The group may be selected to facilitate bonding with the linker or to improve the activity of the CDK8/19 degrader. As demonstrated in the Examples, the choice of the group, such as one comprising piperazine, may improve activity. Exemplary groups used to prepare the CDK8/19 degraders included, without limitation,

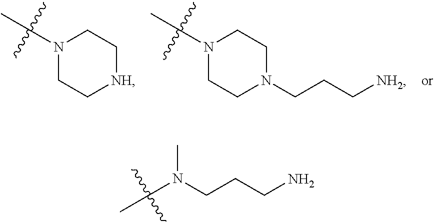

but other groups may also be used.

In some embodiments, the linker and ubiquitin ligase ligand moiety are selected together to optimize degradation. Each time the degrader recruits a different ligase, the conformational requirements that allow for ubiquitinoylation change and the selection of the linker may be chosen to increase the likelihood that the POI is ubiquitinoylated.

The degraders described herein may be a prodrug. Prodrugs are derivatives of an active drug designed to ameliorate an undesirable physical or biological property. The physical properties are usually solubility (too much or not enough lipid or aqueous solubility) or stability related, while problematic biological properties include too rapid metabolism or poor bioavailability which itself may be related to a physicochemical property. Prodrugs are usually prepared by: a) formation of ester, hemi esters, carbonate esters, nitrate esters, amides, hydroxamic acids, carbamates, imines, Mannich bases, phosphates, phosphate esters, and enamines of the active drug, b) functionalizing the drug with azo, glycoside, peptide, and ether functional groups, c) use of aminals, hemi-aminals, polymers, salts, complexes, phosphoramides, acetals, hemiacetals, and ketal forms of the drug. For example, see Andrejus Korolkovas's, “Essentials of Medicinal Chemistry”, John Wiley-Interscience Publications, John Wiley and Sons, New York (1988), pp. 97-118. As demonstrated in the Examples, a prodrug prepared with a methyl pivalate group was a highly potent degrader.

Exemplary CD8/19 degraders and methods for making the degraders are provided in the Examples.

The CDK8/19 degraders disclosed herein may be formulated as a pharmaceutical composition comprising an effective amount of one or more compounds and one or more pharmaceutically acceptable carriers, excipients, or diluents. The pharmaceutical composition may include the CDK8/19 degrader in a range of about 0.1 to 2000 mg (preferably about 0.5 to 500 mg, and more preferably about 1 to 100 mg). The pharmaceutical composition may be administered to provide the compound at a daily dose of about 0.1 to 100 mg/kg body weight (preferably about 0.5 to 20 mg/kg body weight, more preferably about 0.1 to 10 mg/kg body weight). In some embodiments, after the pharmaceutical composition is administered to a patient (e.g., after about 1, 2, 3, 4, 5, or 6 hours post-administration), the concentration of the compound at the site of action is about 1 nM to 1 μM.

The CDK8/19 degraders utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The CDK8/19 degraders utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The CDK8/19 degraders utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents.

Suitable diluents may include pharmaceutically acceptable inert fillers.

The CDK8/19 degraders utilized in the methods disclosed herein may be formulated as a pharmaceutical composition for delivery via any suitable route. For example, the pharmaceutical composition may be administered via oral, intravenous, intramuscular, subcutaneous, topical, and pulmonary route. Examples of pharmaceutical compositions for oral administration include capsules, syrups, concentrates, powders and granules.

The CDK8/19 degraders utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The CDK8/19 degraders employed in the compositions and methods disclosed herein may be administered as pharmaceutical compositions and, therefore, pharmaceutical compositions incorporating the compounds are considered to be embodiments of the compositions disclosed herein. Such compositions may take any physical form, which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures. The compounds for use according to the methods disclosed herein may be administered as a single compound or a combination of compounds.

As indicated above, pharmaceutically acceptable salts of the compounds are contemplated and also may be utilized in the disclosed methods. The term “pharmaceutically acceptable salt” as used herein, refers to salts of the compounds which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Pharmaceutically acceptable esters and amides of the compounds can also be employed in the compositions and methods disclosed herein.

In addition, the methods disclosed herein may be practiced using solvate forms of the compounds or salts, esters, and/or amides, thereof. Solvate forms may include ethanol solvates, hydrates, and the like.

### Methods of Treatment

Methods for treating subjects with a CDK8/19 degrader are provided. Suitably the method for treating a subject comprises administering to the subject an effective amount of a CDK8/19 degrader. As used herein the term “effective amount” refers to the amount or dose of the compound that provides the desired effect. In some embodiments, the effective amount is the amount or dose of the compound, upon single or multiple dose administration to the subject, which provides the desired effect in the subject under diagnosis or treatment. The desired effect may be treatment of a subject in need of a degrader for CDK8 and/or CDK19, degradation of CDK8 and/or CDK19, or having a disease disorder or condition that is dependent on CDK8 and/or CDK19.

In some embodiments, the effective amount is an amount or dose of the compounds that provides an anticancer effect. An anticancer effect may be inhibition of the growth or proliferation of a cancer cell. In some embodiments, the anticancer effect is the inhibiting the growth or proliferation of a blood (e.g., leukemia, multiple myeloma or lymphoma), prostate, breast, colon, nervous system, or osteosarcoma cancer cell.

In some embodiments, the effective amount is an amount or dose of compounds that prevents or reduces the induction of genes that promote metastasis.

In some embodiments, the effective amount is an amount or dose of compounds that prevents or reduces the induction of genes that promote drug resistance in cancer cells. In some embodiments, the subject is undergoing treatment with an anticancer agent, such as a DNA-damaging chemotherapeutic agent or radiation, or a drug targeting a signal transduction pathway critical for the cancer, and co-administration of any of the CDK8/19 degraders described here reduces resistance with the anticancer agent or sensitizes the cancer to the anticancer agent. Resistance to both conventional and targeted drugs involves metastable transcriptional changes that allow tumor cells to adapt and survive drug exposure. Transcriptional reprogramming is a key feature of tumor cell plasticity, which allows the cells to grow under adversarial conditions (treatment resistance) and in a heterologous environment (metastasis). This non-genetic resistance of tumor cell populations provides the background for subsequent selection of stable genetic variants that yield higher levels of resistance. Enhancement to anticancer therapy may be realized by administering a CDK8/19 degrader. In some embodiments, the subject is responsive to therapy with one or more of the CDK8/19 degraders disclosed herein in combination with one or more additional therapeutic agents. The additional therapeutic agent may be administered at the same time in a single composition or as separate compounds or composition. In other embodiments, the additional therapeutic agent may be administered at a different time as the CDK8/19 degrader.

In some embodiments, the effective amount is an amount that reduces inflammation in the subject having an inflammation-associated disease.

As used herein, a “subject” may be interchangeable with “patient” or “individual” and means an animal, which may be a human or non-human animal, in need of treatment. In particular embodiments, the subject is a human subject. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

A “subject in need of treatment” may include a subject having a disease, disorder, or condition that dependent of CDK8 and/or CDK19. A “subject in need of treatment” may include a subject having a disease, disorder, or condition that is dependent on CCNC.

In some embodiments, the disease, disorder, or condition is a cancer. In some embodiments, the cancer is a blood, prostate, breast, colon, nervous system, or osteo cancer. In some embodiments, the subject has multiple myeloma, castration-refractory prostate cancer (CRPC), acute myeloid leukemia, hepatic metastases of colon cancer, estrogen receptor-positive breast cancer, or HER2-positive breast cancer. In some embodiments, the subject has a cancer (e.g., multiple myeloma) that is dependent on Cyclin C.

In some embodiments, the subject has an inflammation-associated disease.

In some embodiments, the subject has a cardiovascular disease.

In some embodiments, the subject has a ribosomopathy.

In some embodiments, the subject has a condition characterized by reduced number of hematopoietic stem cells and/or progenitor cells.

In some embodiments, the subject has a bone anabolic disorder.

In some embodiments, the subject has a viral disease.

An effective amount can be readily determined by those of skill in the art, including an attending diagnostician, by the use of known techniques and by observing results obtained under analogous circumstances. In determining the effective amount or dose of compound administered, a number of factors can be considered by the attending diagnostician, such as: the species of the subject, its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent depending on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1. Synthesis and Structure-Activity Relationship (SAR) Analysis of PROTACs Targeting CDK8 and CDK19

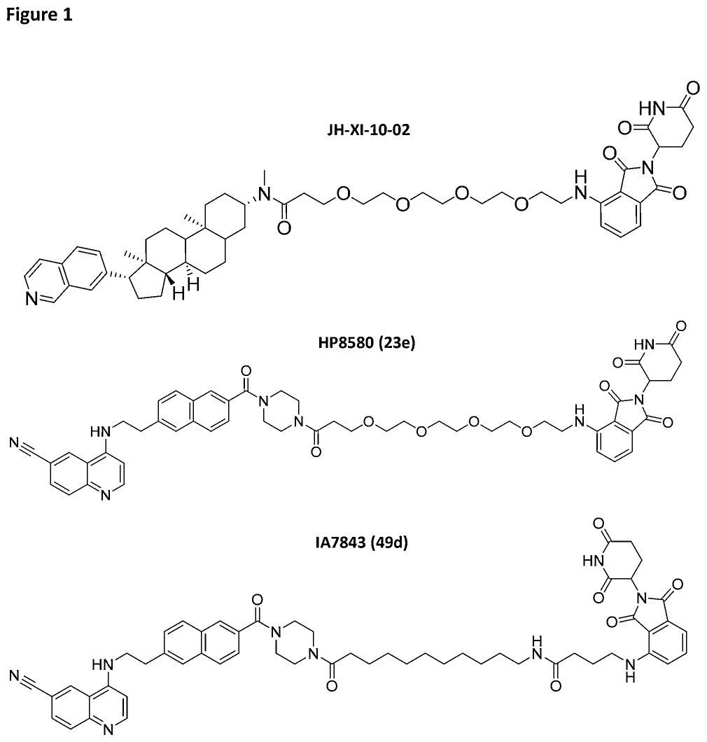

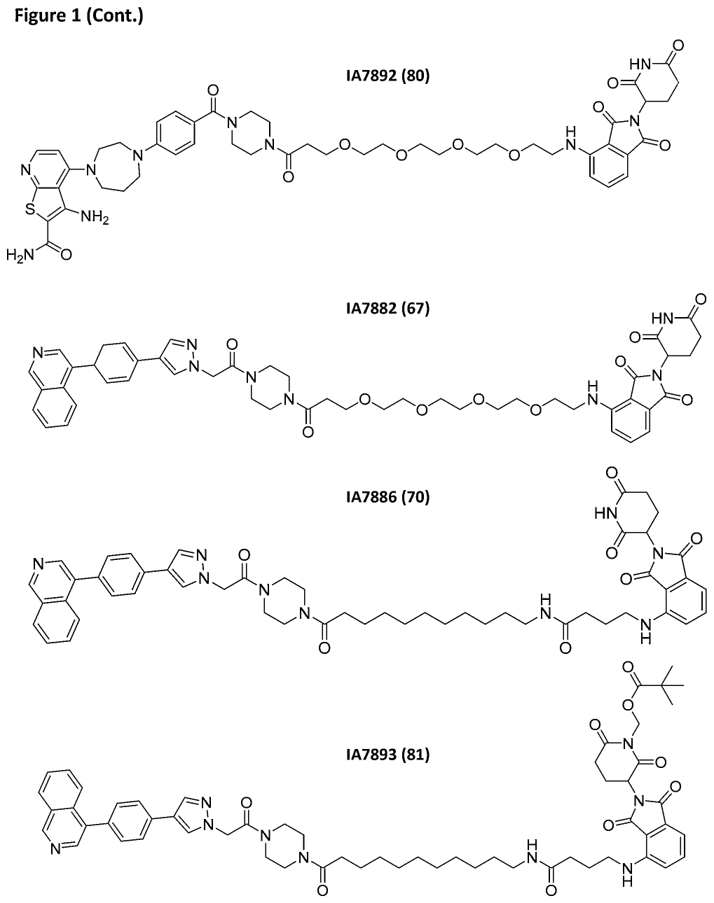

CDK8/19 degraders were prepared from chemically distinct CDK8/19 kinase inhibitors, 15u (PCT/US2020/033937), Senexin C (U.S. Pat. No. 11,014,906), and BI-1347 (Hofmann et al., 2020), all of which bind to the ATP pocket of CDK8. JH-XI-10-02 (obtained from MedChemExpress) was also tested, which is based on a different CDK8 ATP pocket binder, JH-VIII-49 (Hatcher et al., 2018). These CDK8-targeting moieties were connected with different ubiquitin E3 ligase targeting moieties (e.g., CRBN E3 ligase ligand pomalidomide, von Hippel-Lindau (VHL) VHL ligand VH032 or DCAF 16 E3 ligase ligand KB02) via different polyethylene glycol or long-chain alkane linkers. Structures of the most potent PROTACs based on different CDK8/19 kinase inhibitors, as well as the previously reported PROTAC JH-XI-10-02 (Hatcher et al., 2018) are shown in FIG. 1.

Table 1 presents the structures of 35 different chimeras that have been synthesized, together with their effects (at 1 μM or 0.3 μM concentrations) on CDK8 degradation in HEK-293 cells. The synthesis schemes of all the compounds are described below. CDK8 degradation was measured by Western blot analysis of RIPA lysates of HEK-293cells treated with or without the test compound for 24 hours, using antibodies against CDK8 (Santa Cruz sc-1521) and housekeeping protein GAPDH (Santa Cruz sc-32233) (loading control) Protein bands on immunoblotted PVDF membranes were visualized with Western Lighting Plus ECL detection reagent (Perkin Elmer, Waltham, MA, USA) using ChemiDoc Touch™ (Bio-Rad). Image processing and densitometry analysis were performed using ImageLab software (Bio-Rad) to quantify the CDK8 protein levels (normalized by GAPDH) and evaluate the effects of compound treatment.

TABLE 1
Structures and CDK8-degradation activities of CDK8/19 degraders.

| | | | Chemical | % CDK8 |
|---|---|---|---|---|
| ID | CMPD ID | Chemical Structure | formula | remaining |
| 23a | HP8581 | 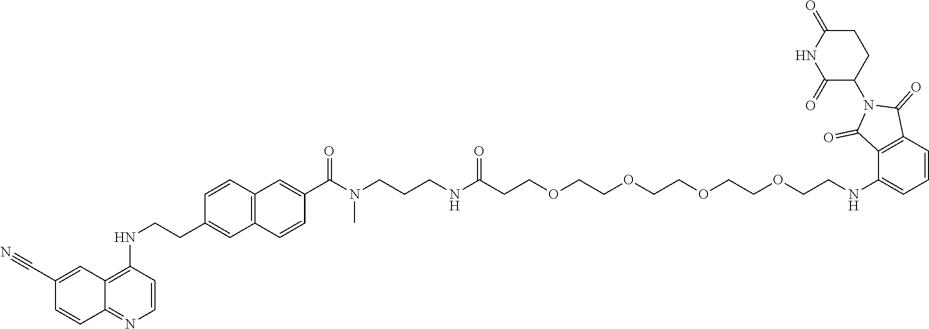 [SMILES: CN(CCCNC(=O)CCOCCOCCOCCOCCNc1cccc2c1C(=O)N(C1CCC(=O)NC1=O)C2=O)C(=O)c1ccc2cc(CCNc3ccnc4ccc(C#N)cc34)ccc2c1] | C51H56N8O10 | 36% |
| 23b | HP8579 | 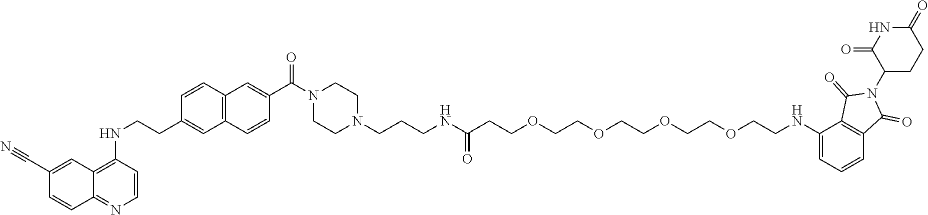 [SMILES: N#Cc1ccc2nccc(NCCc3ccc4cc(C(=O)N5CCN(CCCNC(=O)CCOCCOCCOCCOCCNc6cccc7c6C(=O)N(C6CCC(=O)NC6=O)C7=O)CC5)ccc4c3)c2c1] | C54H61N9O10 | 29% |
| 23c | HV5958 | 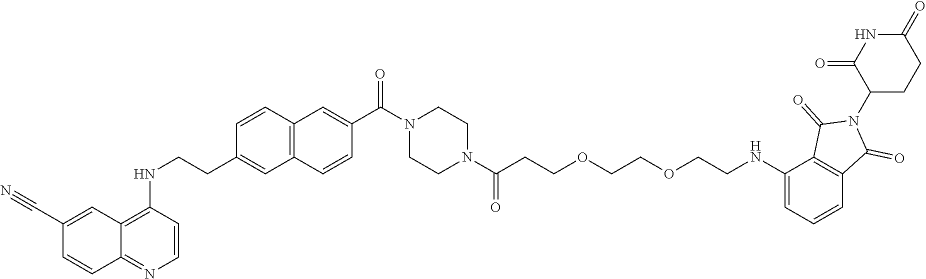 [SMILES: N#Cc1ccc2nccc(NCCc3ccc4cc(C(=O)N5CCN(C(=O)CCOCCOCCNc6cccc7c6C(=O)N(C6CCC(=O)NC6=O)C7=O)CC5)ccc4c3)c2c1] | C47H46N8O8 | 27% |
| 23d | HV5957 | 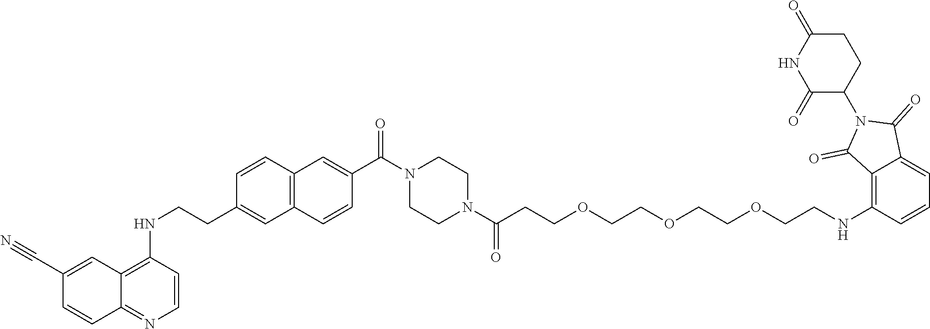 [SMILES: N#Cc1ccc2nccc(NCCc3ccc4cc(C(=O)N5CCN(C(=O)CCOCCOCCOCCNc6cccc7c6C(=O)N(C6CCC(=O)NC6=O)C7=O)CC5)ccc4c3)c2c1] | C49H50N8O9 | 21% |
| 23e | HP8580 | 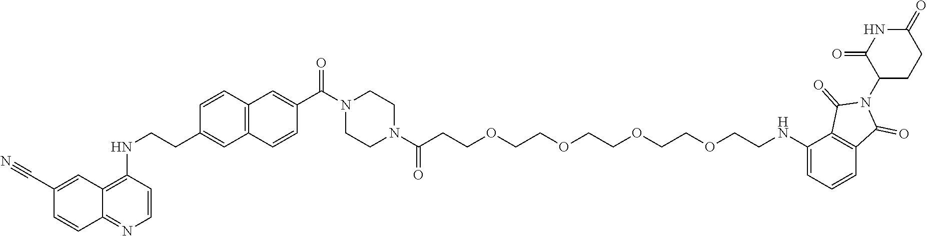 [SMILES: N#Cc1ccc2nccc(NCCc3ccc4cc(C(=O)N5CCN(C(=O)CCOCCOCCOCCOCCNc6cccc7c6C(=O)N(C6CCC(=O)NC6=O)C7=O)CC5)ccc4c3)c2c1] | C51H54N8O10 | 20% |
| 23f | IA7812 | 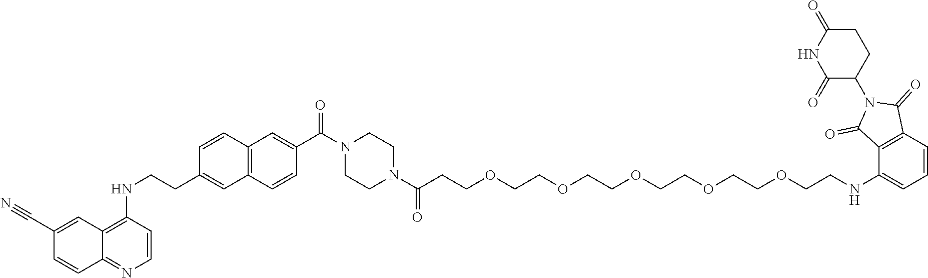 [SMILES: N#Cc1ccc2nccc(NCCc3ccc4cc(C(=O)N5CCN(C(=O)CCOCCOCCOCCOCCOCCNc6cccc7c6C(=O)N(C6CCC(=O)NC6=O)C7=O)CC5)ccc4c3)c2c1] | C53H58N8O11 | 29% |
| 23g | IA7813 | 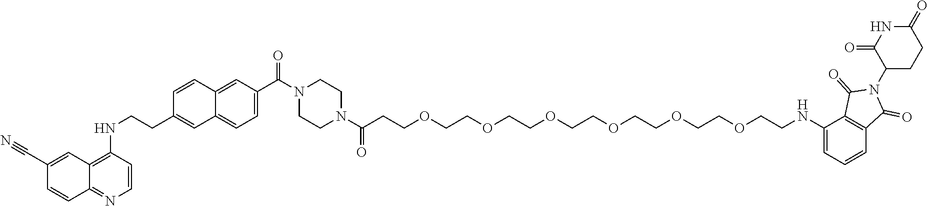 [SMILES: N#Cc1ccc2nccc(NCCc3ccc4cc(C(=O)N5CCN(C(=O)CCOCCOCCOCCOCCOCCOCCNc6cccc7c6C(=O)N(C6CCC(=O)NC6=O)C7=O)CC5)ccc4c3)c2c1] | C55H62N8O12 | 28% |
| 25 | IA7807 | 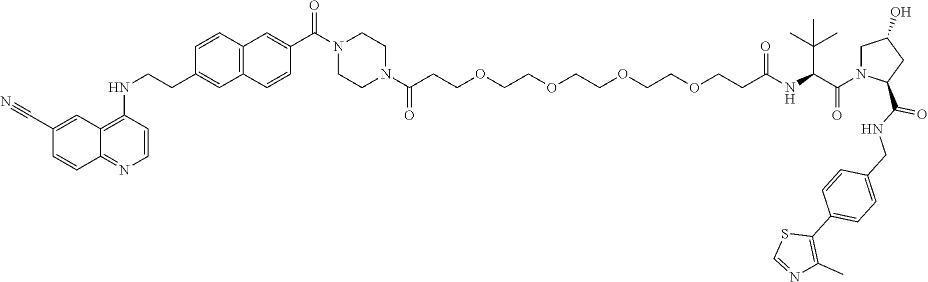 [SMILES: Cc1ncsc1-c1ccc(CNC(=O)[C@@H]2C[C@@H](O)CN2C(=O)[C@@H](NC(=O)CCOCCOCCOCCOCCC(=O)N2CCN(C(=O)c3ccc4cc(CCNc5ccnc6ccc(C#N)cc56)ccc4c3)CC2)C(C)(C)C)cc1] | C61H73N9O10S | 100% |
| 28a | IA7819 | 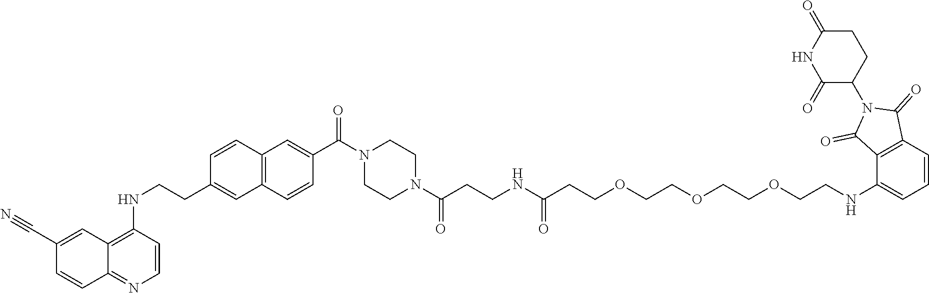 [SMILES: N#Cc1ccc2nccc(NCCc3ccc4cc(C(=O)N5CCN(C(=O)CCNC(=O)CCOCCOCCOCCNc6cccc7c6C(=O)N(C6CCC(=O)NC6=O)C7=O)CC5)ccc4c3)c2c1] | C52H55N9O10 | 66% |
| 28b | IA7822 | 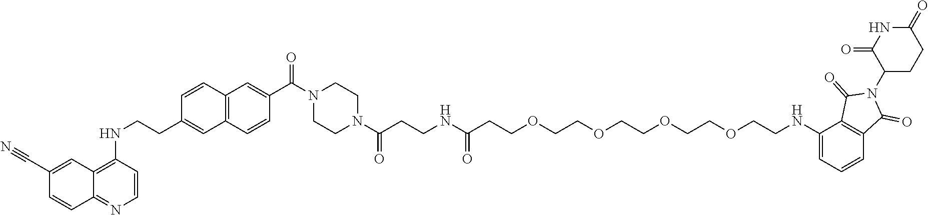 [SMILES: N#Cc1ccc2nccc(NCCc3ccc4cc(C(=O)N5CCN(C(=O)CCNC(=O)CCOCCOCCOCCOCCNc6cccc7c6C(=O)N(C6CCC(=O)NC6=O)C7=O)CC5)ccc4c3)c2c1] | C54H59N9O11 | 65% |
| 32 | IA7803 | 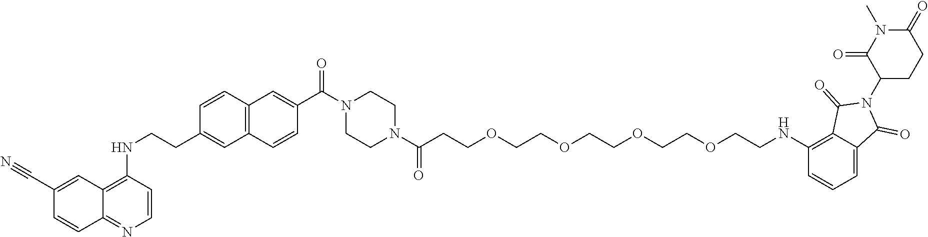 [SMILES: CN1C(=O)CCC(N2C(=O)c3cccc(NCCOCCOCCOCCOCCC(=O)N4CCN(C(=O)c5ccc6cc(CCNc7ccnc8ccc(C#N)cc78)ccc6c5)CC4)c3C2=O)C1=O] | C52H56N8O10 | 100% |
| 43 | IA7830 | 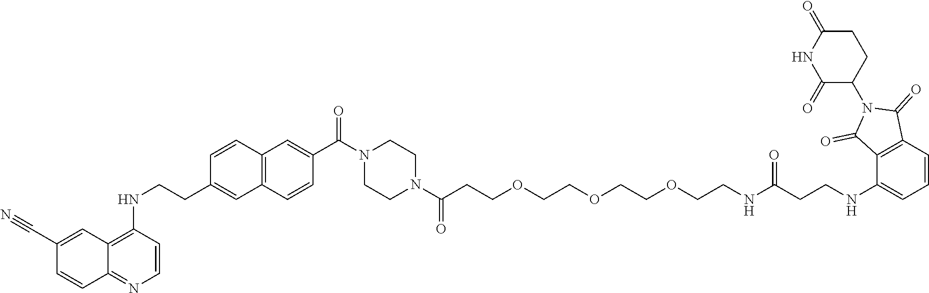 [SMILES: N#Cc1ccc2nccc(NCCc3ccc4cc(C(=O)N5CCN(C(=O)CCOCCOCCOCCNC(=O)CCNc6cccc7c6C(=O)N(C6CCC(=O)NC6=O)C7=O)CC5)ccc4c3)c2c1] | C52H55N9O10 | 65% |
| 46 | IA7835 | 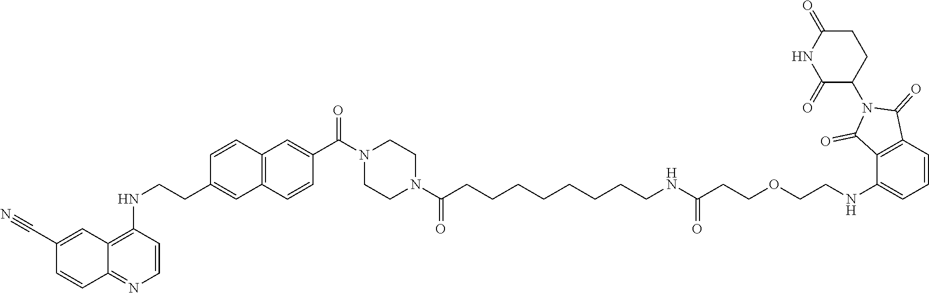 [SMILES: N#Cc1ccc2nccc(NCCc3ccc4cc(C(=O)N5CCN(C(=O)CCCCCCCCNC(=O)CCOCCNc6cccc7c6C(=O)N(C6CCC(=O)NC6=O)C7=O)CC5)ccc4c3)c2c1] | C54H59N9O8 | 45% |
| 47 | IA7840 | 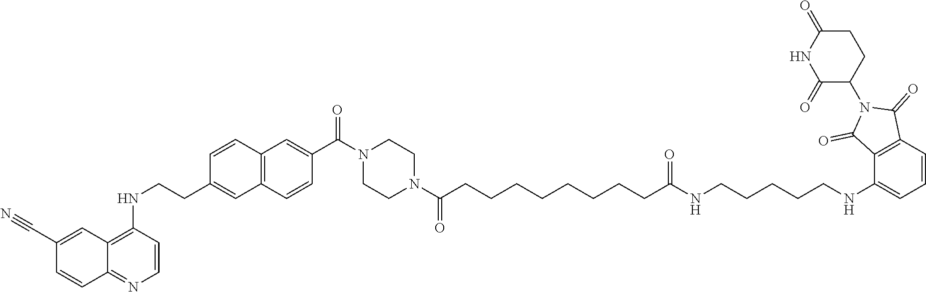 [SMILES: N#Cc1ccc2nccc(NCCc3ccc4cc(C(=O)N5CCN(C(=O)CCCCCCCCC(=O)NCCCCCNc6cccc7c6C(=O)N(C6CCC(=O)NC6=O)C7=O)CC5)ccc4c3)c2c1] | C55H61N9O7 | 55% |
| 48 | IA7862 | 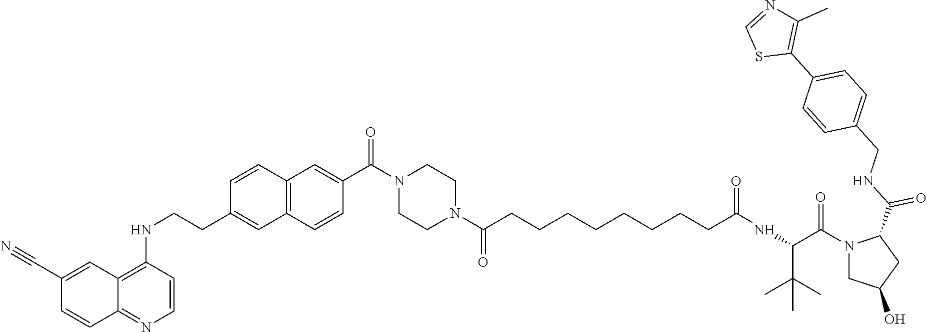 [SMILES: Cc1ncsc1-c1ccc(CNC(=O)[C@@H]2C[C@@H](O)CN2C(=O)[C@@H](NC(=O)CCCCCCCCC(=O)N2CCN(C(=O)c3ccc4cc(CCNc5ccnc6ccc(C#N)cc56)ccc4c3)CC2)C(C)(C)C)cc1] | C59H69N9O6S | 69% |
| 49a | IA7858 | 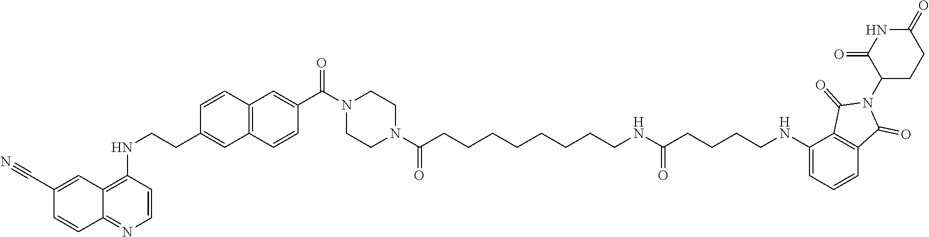 [SMILES: N#Cc1ccc2nccc(NCCc3ccc4cc(C(=O)N5CCN(C(=O)CCCCCCCCNC(=O)CCCCNc6cccc7c6C(=O)N(C6CCC(=O)NC6=O)C7=O)CC5)ccc4c3)c2c1] | C54H59N9O7 | 21% |
| 49b | IA7859 | 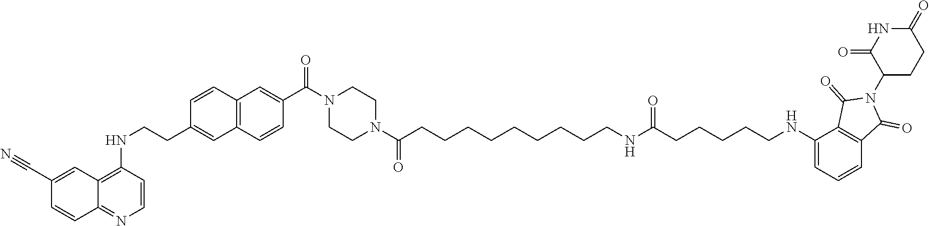 [SMILES: N#Cc1ccc2nccc(NCCc3ccc4cc(C(=O)N5CCN(C(=O)CCCCCCCCCNC(=O)CCCCCNc6cccc7c6C(=O)N(C6CCC(=O)NC6=O)C7=O)CC5)ccc4c3)c2c1] | C56H63N9O | 81% |
| 49c | IA7860 | 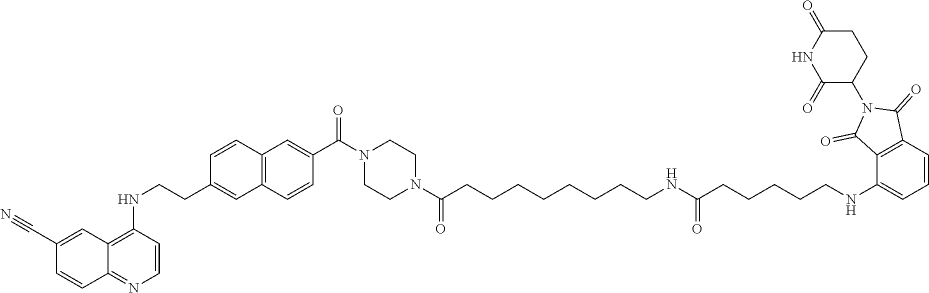 [SMILES: N#Cc1ccc2nccc(NCCc3ccc4cc(C(=O)N5CCN(C(=O)CCCCCCCCNC(=O)CCCCCNc6cccc7c6C(=O)N(C6CCC(=O)NC6=O)C7=O)CC5)ccc4c3)c2c1] | C55H6IN9O7 | 95% |
| 49d | IA7843 | 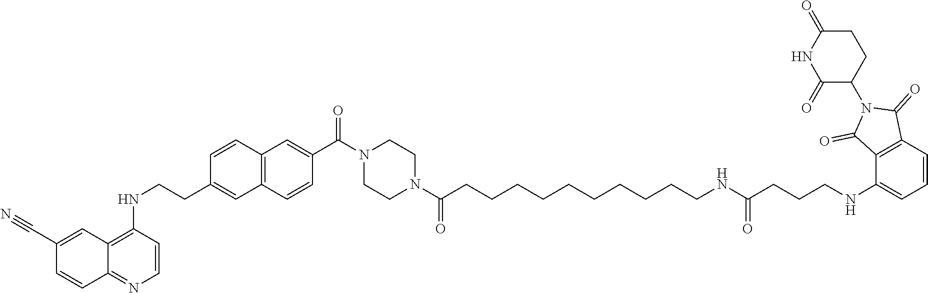 [SMILES: N#Cc1ccc2nccc(NCCc3ccc4cc(C(=O)N5CCN(C(=O)CCCCCCCCCCNC(=O)CCCNc6cccc7c6C(=O)N(C6CCC(=O)NC6=O)C7=O)CC5)ccc4c3)c2c1] | C55H6IN9O7 | 13% |
| 49e | IA7848 | 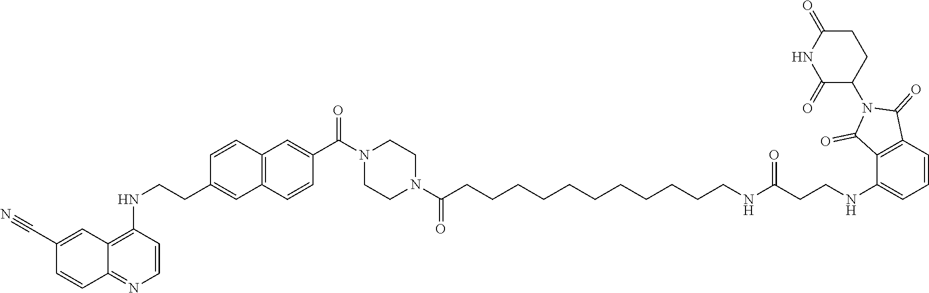 [SMILES: N#Cc1ccc2nccc(NCCc3ccc4cc(C(=O)N5CCN(C(=O)CCCCCCCCCCCNC(=O)CCNc6cccc7c6C(=O)N(C6CCC(=O)NC6=O)C7=O)CC5)ccc4c3)c2c1] | C55H61N9O7 | 31% |
| 49f | IA7861 | 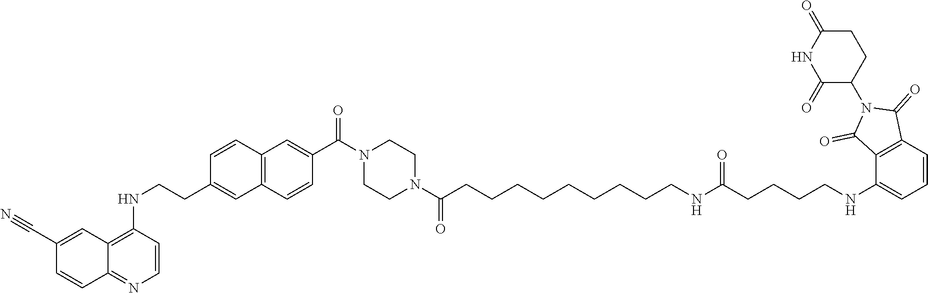 [SMILES: N#Cc1ccc2nccc(NCCc3ccc4cc(C(=O)N5CCN(C(=O)CCCCCCCCCNC(=O)CCCCNc6cccc7c6C(=O)N(C6CCC(=O)NC6=O)C7=O)CC5)ccc4c3)c2c1] | C55H61N9O7 | 19% |
| 53 | IA7868 | 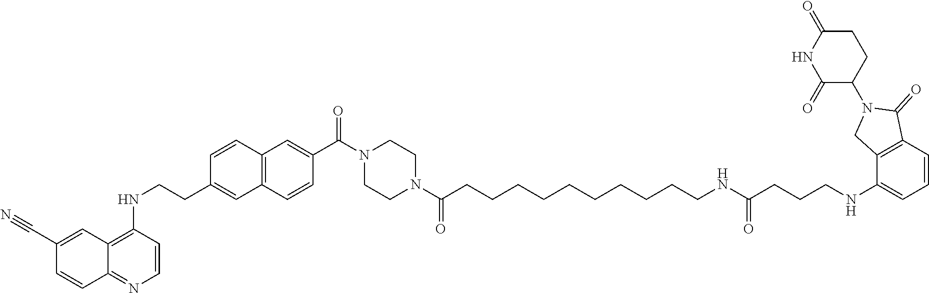 [SMILES: N#Cc1ccc2nccc(NCCc3ccc4cc(C(=O)N5CCN(C(=O)CCCCCCCCCCNC(=O)CCCNc6cccc7c6CN(C6CCC(=O)NC6=O)C7=O)CC5)ccc4c3)c2c1] | C55H61N9O7 | 39% |
| 56 | IA7869 | 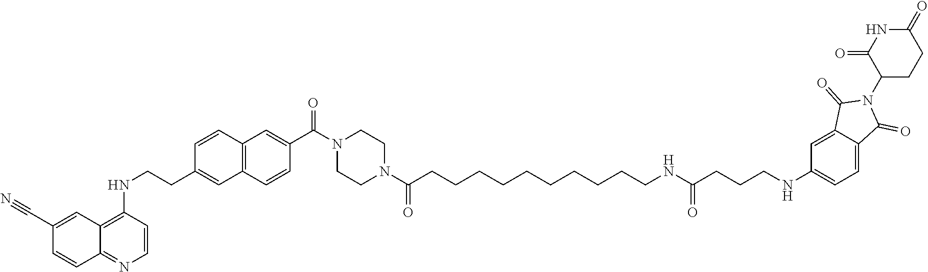 [SMILES: N#Cc1ccc2nccc(NCCc3ccc4cc(C(=O)N5CCN(C(=O)CCCCCCCCCCNC(=O)CCCNc6ccc7c(c6)C(=O)N(C6CCC(=O)NC6=O)C7=O)CC5)ccc4c3)c2c1] | C55H63N9O6 | 43% |
| 60 | IA7875 | 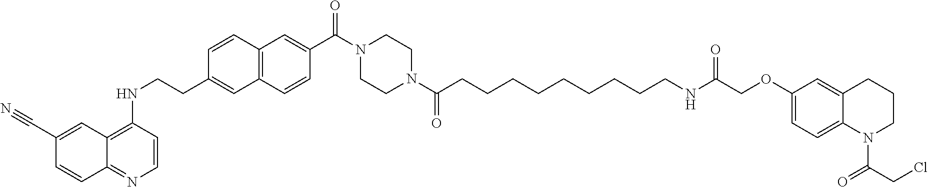 [SMILES: N#Cc1ccc2nccc(NCCc3ccc4cc(C(=O)N5CCN(C(=O)CCCCCCCCCNC(=O)COc6ccc7c(c6)CCCN7C(=O)CCl)CC5)ccc4c3)c2c1] | C50H56ClN7O5 | 100% |
| 80 | IA7892 | 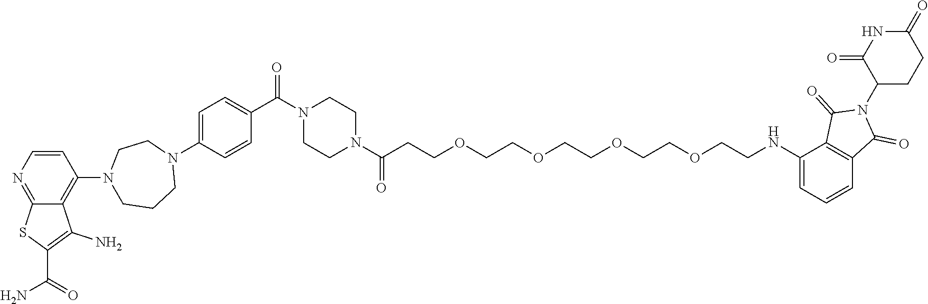 [SMILES: NC(=O)c1sc2nccc(N3CCCN(c4ccc(C(=O)N5CCN(C(=O)CCOCCOCCOCCOCCNc6cccc7c6C(=O)N(C6CCC(=O)NC6=O)C7=O)CC5)cc4)CC3)c2c1N] | C48H58N10O11S | 41% |
| 24a | HP8578 | 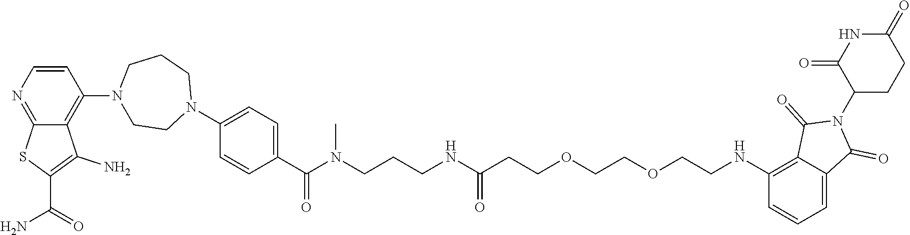 [SMILES: CN(CCCNC(=O)CCOCCOCCNc1cccc2c1C(=O)N(C1CCC(=O)NC1=O)C2=O)C(=O)c1ccc(N2CCCN(c3ccnc4sc(C(N)=O)c(N)c34)CC2)cc1] | C44H52N10O9S | 82% |
| 24b | HP8575 | 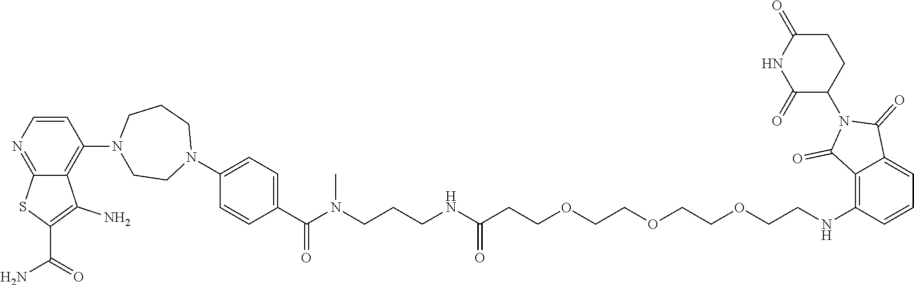 [SMILES: CN(CCCNC(=O)CCOCCOCCOCCNc1cccc2c1C(=O)N(C1CCC(=O)NC1=O)C2=O)C(=O)c1ccc(N2CCCN(c3ccnc4sc(C(N)=O)c(N)c34)CC2)cc1] | C46H56N10O10S | 77% |
| 24c | HP8553 | 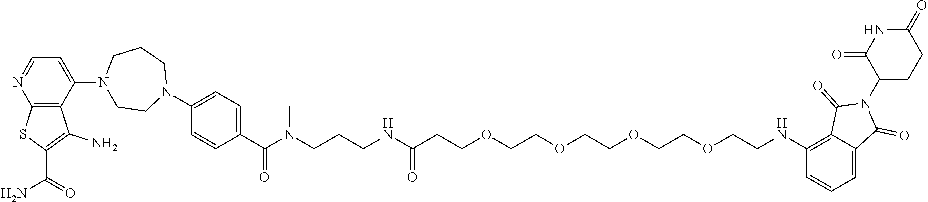 [SMILES: CN(CCCNC(=O)CCOCCOCCOCCOCCNc1cccc2c1C(=O)N(C1CCC(=O)NC1=O)C2=O)C(=O)c1ccc(N2CCCN(c3ccnc4sc(C(N)=O)c(N)c34)CC2)cc1] | C48H60N10O11S | 74% |
| 24d | IA7814 | 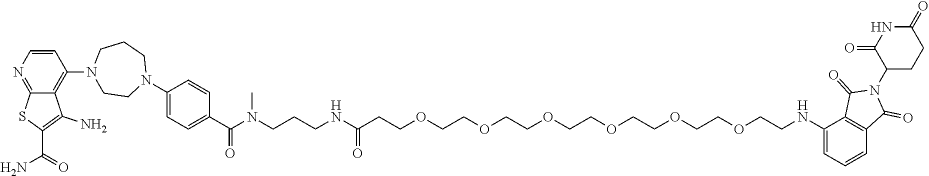 [SMILES: CN(CCCNC(=O)CCOCCOCCOCCOCCOCCOCCNc1cccc2c1C(=O)N(C1CCC(=O)NC1=O)C2=O)C(=O)c1ccc(N2CCCN(c3ccnc4sc(C(N)=O)c(N)c34)CC2)cc1] | C52H68N10O13S | 59% |
| 67 | IA7882 | 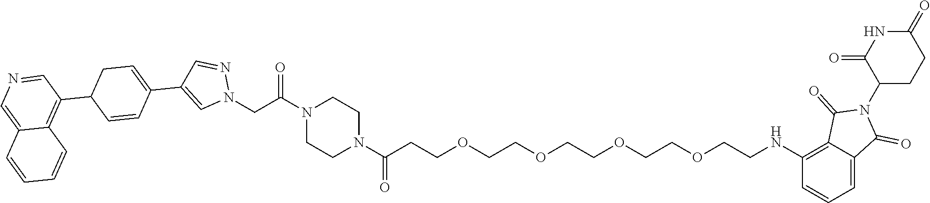 [SMILES: O=C1CCC(N2C(=O)c3cccc(NCCOCCOCCOCCOCCC(=O)N4CCN(C(=O)Cn5cc(C6=CCC(c7cncc8ccccc78)C=C6)cn5)CC4)c3C2=O)C(=O)N1] | C48H54N8O10 | 12% |
| 70 | IA7886 | 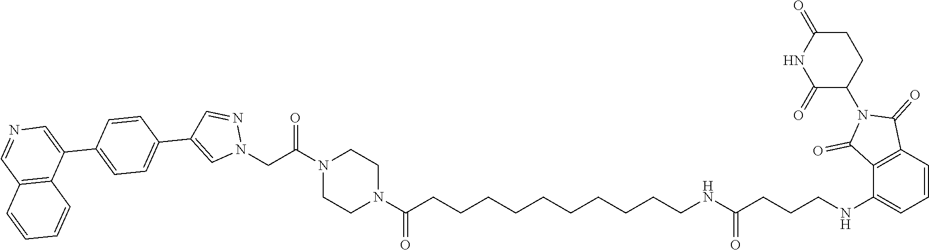 [SMILES: O=C(CCCNc1cccc2c1C(=O)N(C1CCC(=O)NC1=O)C2=O)NCCCCCCCCCCC(=O)N1CCN(C(=O)Cn2cc(-c3ccc(-c4cncc5ccccc45)cc3)cn2)CC1] | C52H59N9O7 | 19% |
| 73 | IA7891 | 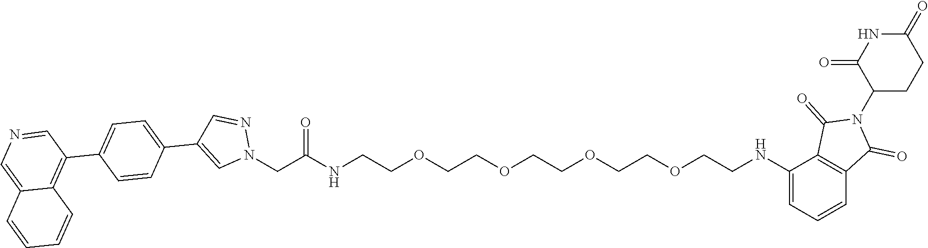 [SMILES: O=C(Cn1cc(-c2ccc(-c3cncc4ccccc34)cc2)cn1)NCCOCCOCCOCCOCCNc1cccc2c1C(=O)N(C1CCC(=O)NC1=O)C2=O] | C43H45N7O9 | 100% |
| 81 | IA7893 | 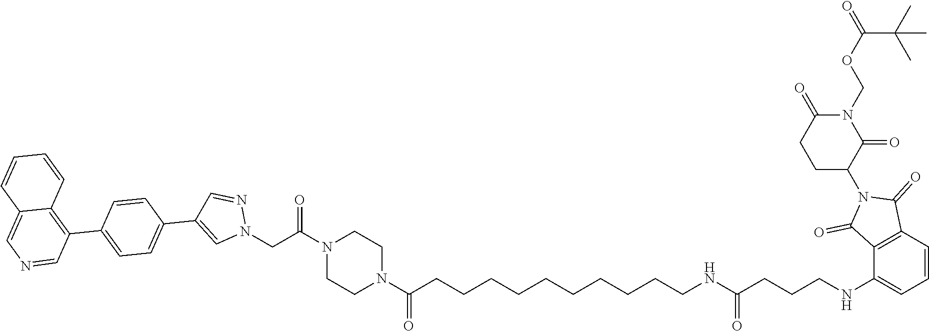 [SMILES: CC(C)(C)C(=O)OCN1C(=O)CCC(N2C(=O)c3cccc(NCCCC(=O)NCCCCCCCCCCC(=O)N4CCN(C(=O)Cn5cc(-c6ccc(-c7cncc8ccccc78)cc6)cn5)CC4)c3C2=O)C1=O] | C58H69N9O9 | 9%* |
| 82 | IA7894 | 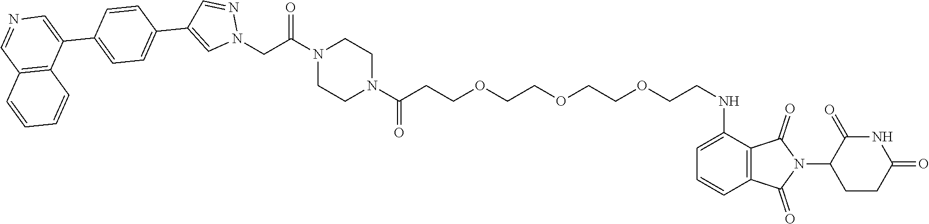 [SMILES: O=C1CCC(N2C(=O)c3cccc(NCCOCCOCCOCCC(=O)N4CCN(C(=O)Cn5cc(-c6ccc(-c7cncc8ccccc78)cc6)cn5)CC4)c3C2=O)C(=O)N1] | C46H48N8O9 | 51%* |
| 83 | IA7895 | 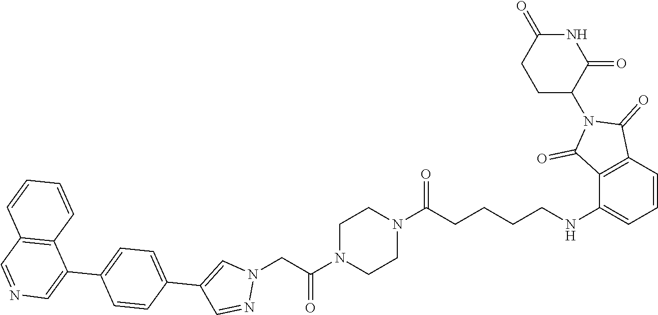 [SMILES: O=C1CCC(N2C(=O)c3cccc(NCCCCC(=O)N4CCN(C(=O)Cn5cc(-c6ccc(-c7cncc8ccccc78)cc6)cn5)CC4)c3C2=O)C(=O)N1] | C42H40N8O6 | 70%* |

% CDK8 remaining tested at 1 μM or, when indicated with *, 0.3 μM

The results in Table 1 demonstrate that pomalidomide was the most effective among the E3 ligase moieties tested. The efficacy of CDK8-targeting moieties was BI-1347 (IA 7882, IA 7886, IA7893)>Senexin C (HP8580, IA7843)>15u (IA7892). JH-XI-10-02 was less potent than the lead compounds from the three novel series that we developed (52% CDK8 remaining after 1 μM treatment). Both polyethylene glycol or long-chain alkane linkers yielded efficient PROTACs but the most potent degraders contained long-chain alkane linkers (examples include IA7843 [scheme 49d] vs HP8580 [23e] and IA7886 vs IA7882 [67]). Remarkably, the addition of piperazine group between the CDK8-targeting moiety and the linker drastically increased the CDK8 degradation efficiency in different series, as can be seen by the differences between Senexin C-based compounds (HP8581 [23a] vs HP8580 [23e]), BI1347-based compounds (IA7882 vs IA7891 [73]) and 15u-based compounds (IA 7892 vs HP8553 [24c]).

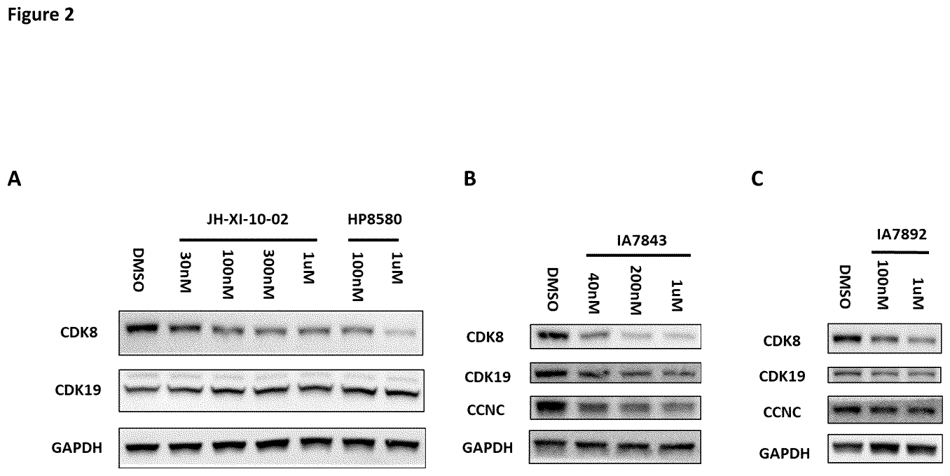

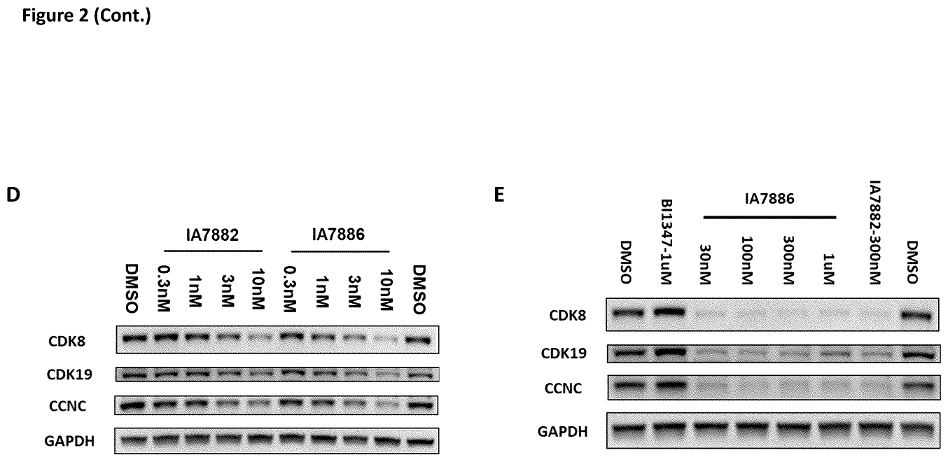

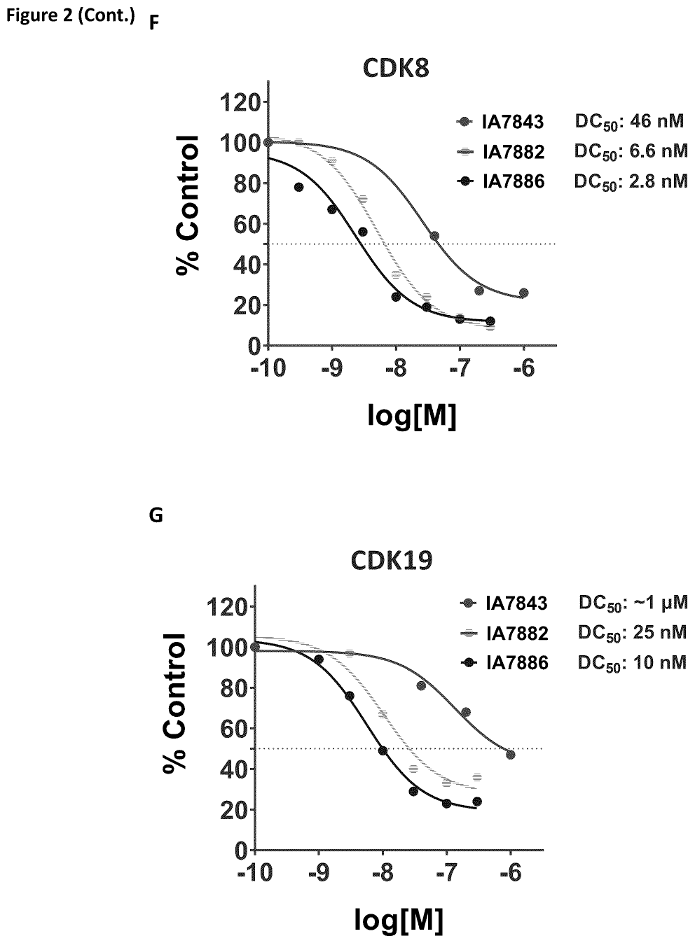

Western blots in FIG. 2 illustrate the effects of the most potent compounds in each of the series on CDK8 and CDK19 degradation in HEK-293cells, including Senexin C-based HP8580 and IA7843, 15u-based IA7892 and BI1347-based IA7882 and IA7886, along with the previously reported JH-XI-10-02. IA7886 was the most potent CDK8 degrader in this set and JH-XI-10-02 was the least potent. Furthermore, both Senexin C-based and BI-1347-based PROTAC compounds induced significant degradation of CDK19 (FIG. 2B, D, E), whereas JH-XI-10-02 had no effect on this protein (FIG. 2A). Hence these new PROTACs are not only more efficient than JH-XI-10-02 but also different in being able to induce degradation of both isoforms of the Mediator kinase. Furthermore, the degradation of both CDK8 and CDK19 by the PROTACs is also seen to decrease the levels of their binding partner cyclin C (CCNC) (FIG. 2B, D, E), which is known to be protected from proteolytic degradation by its interaction with CDK8 (Barette et al., 2001).

FIGS. 2F and 2G show the effects of different concentrations of potent Senexin C- and BI-1347-based PROTACs on CDK8 and CDK19 degradation in HEK-293cells. Based on these assays, we calculated DC₅₀ values that correspond to PROTAC concentrations inducing degradation of CDK8 and CDK19 to the half of protein level in untreated cells. The corresponding CDK8 DC₅₀ values were 46 nM for IA7843, 6.6 nM for IA7882 and 2.8 nM for IA7886, and CDK19 DC₅₀ values were 1 μM for IA7843, 25 nM for IA7882 and 10 nM for IA7886.

IA7893 was designed as a prodrug of IA7886 with increased oral bioavailability by adding a lipophilic methyl pivalate group at active site of CRBN ligands. The synthesis of the resulting IA7893 is shown in scheme 25. Remarkably, IA7893 was a highly potent degrader in HEK-293 cells (Table 1).

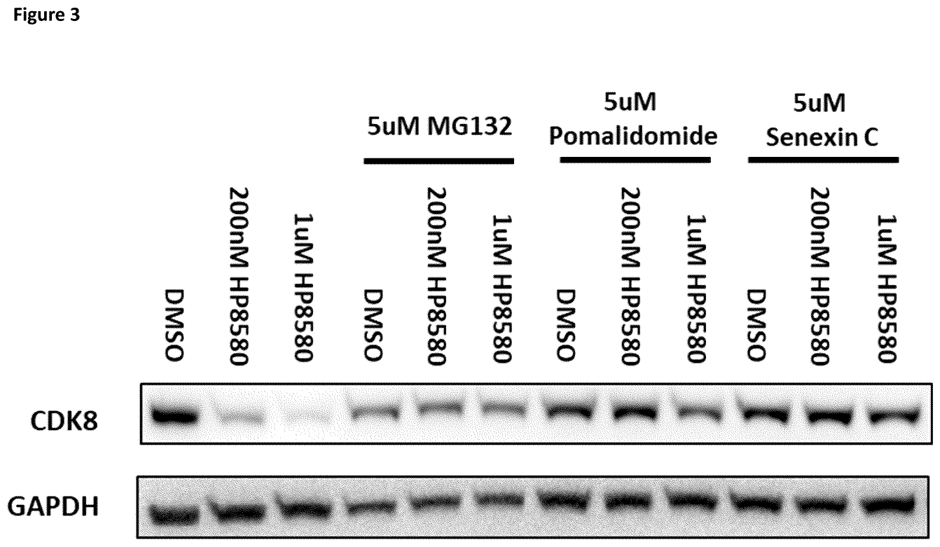

The mechanism of CDK8 degradation by the newly developed PROTACs was verified by the experiment in FIG. 3. This figure compares the effects of Senexin C-based PROTAC HP8580 (200 nM and 1 μM) on CDK8 degradation in HEK-293 cells in the presence or absence of a proteasome inhibitor MG132 (5 μM), 5 μM pomalidomide (which binds E3 ligase but not CDK8) and 5 μM Senexin C (which binds CDK8 but not E3 ligase). Only HP8580 but not pomalidomide or Senexin C induced CDK8 degradation, and the effect of HP8580 was reversed by proteasome inhibition with MG132 as well as by competition with pomalidomide or Senexin C. Hence, HP8580 acts as a bona fide PROTAC.

### Example 2. Novel PROTACs Induce the Degradation of CDK8 and CDK19 in Different Cell Lines and Different Species

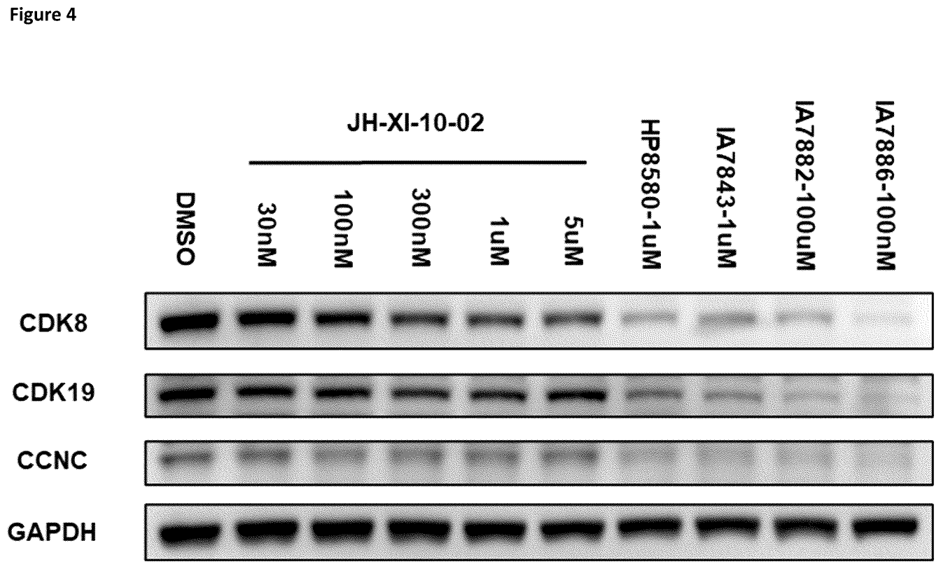

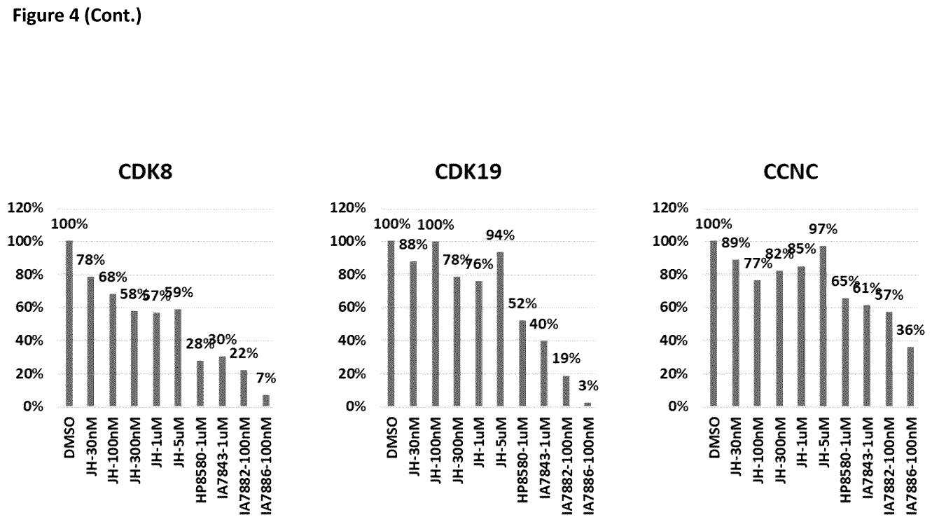

Since the previously published PROTAC JH-XI-10-02 was reported to exert its strongest CDK8 degradation effect in Jurkat leukemia (T-ALL) cells (Hatcher et al., 2018), we have tested this compound in Jurkat leukemia cell line, along with Senexin C-based HP8580 and IA7843 and BI-1347-based IA7882 and IA 7886 (FIG. 4). CDK8 degradation by JH-XI-10-02 was at a similar extent in Jurkat and 293 and the effects of both Senexin C-based and BI-1347-based PROTACs were much stronger than those of JH-XI-10-02. Furthermore, these PROTACs induced CDK19 and CCNC degradation, whereas JH-XI-10-02 had no apparent effect on these proteins.

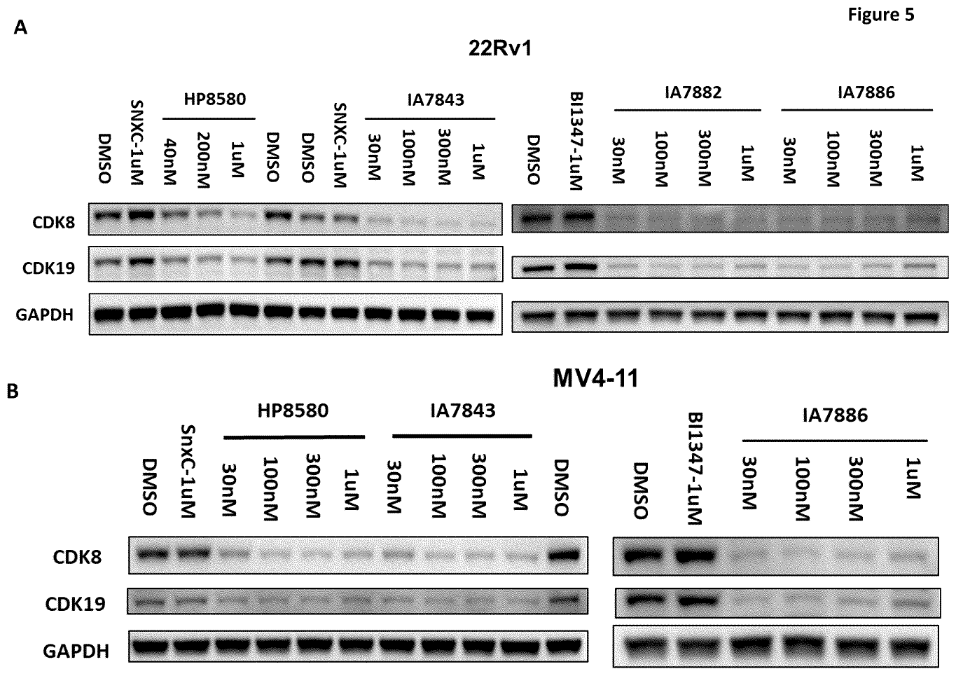

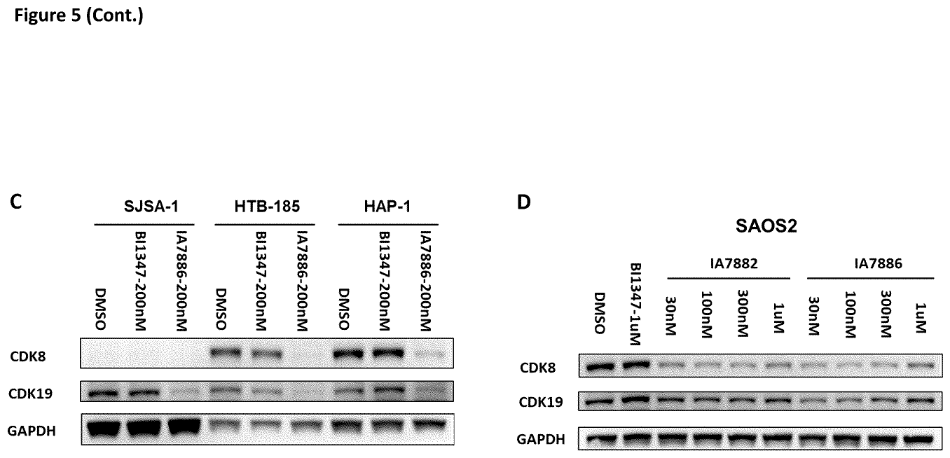

FIG. 5 illustrates the effects of the novel PROTACs HP8580, IA7843 and IA7886 on CDK8 and CDK19 degradation in other human cell lines, including 22Rv1 prostate cancer (expressing more CDK19 than CDK8), MV4-11 (AML) and HAP-1 (CML) leukemia cell lines, HTB-185 medulloblastoma, and SAOS2 and SJSA-1 osteosarcoma (the latter expressing only CDK19 but not CDK8). Although the extent of CDK8 and CDK19 degradation varied between different cell lines, both IA7886 and IA7843 significantly degraded CDK8 and CDK19 in all the tested cell lines.

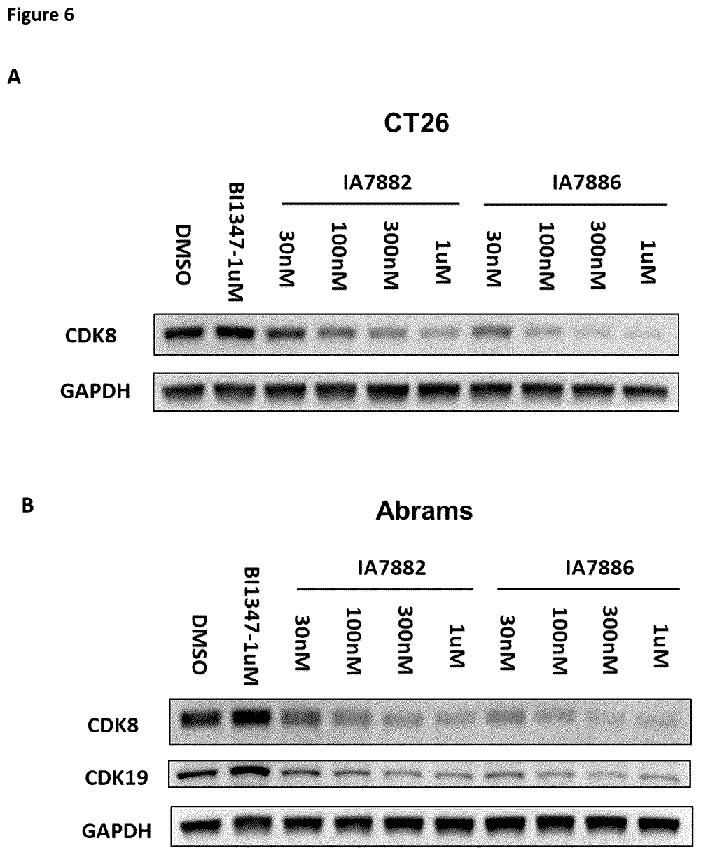

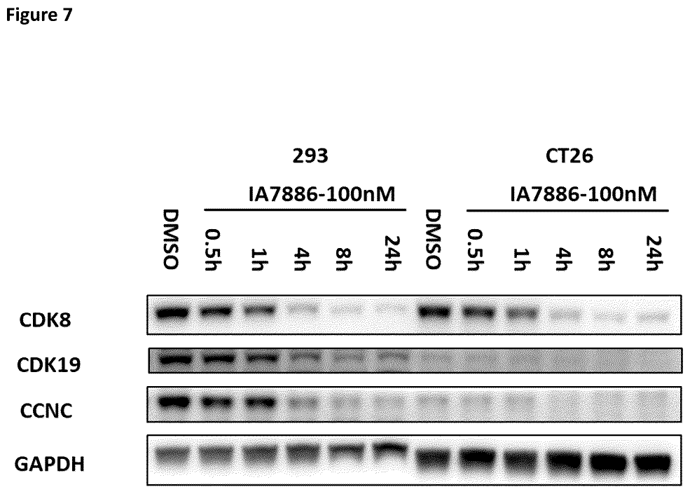

IA 7886 was also tested for the ability to degrade CDK8 and CDK19 in cell lines from other mammalian species, including murine CT26 colon carcinoma cells (FIG. 6A) and canine Abrams osteosarcoma cells (FIG. 6B), and was found to induce CDK8 degradation in both cell lines and induced CDK19 degradation in Abrams cells (CT26 does not express detectable CDK19). FIG. 7 shows the time course of CDK8 degradation by 100 nM IA7886 in human 293 and murine CT26 cells. Both the extent and the kinetics of CDK8 degradation were similar between two cell lines, with the degradation reaching maximum at about 8 hrs of PROTAC treatment.

Hence the novel PROTACs degrade CDK8 and CDK19 in a broad spectrum of cell types and mammals.

### Example 3. Cellular Effects of CDK8/19-Degrading PROTACs

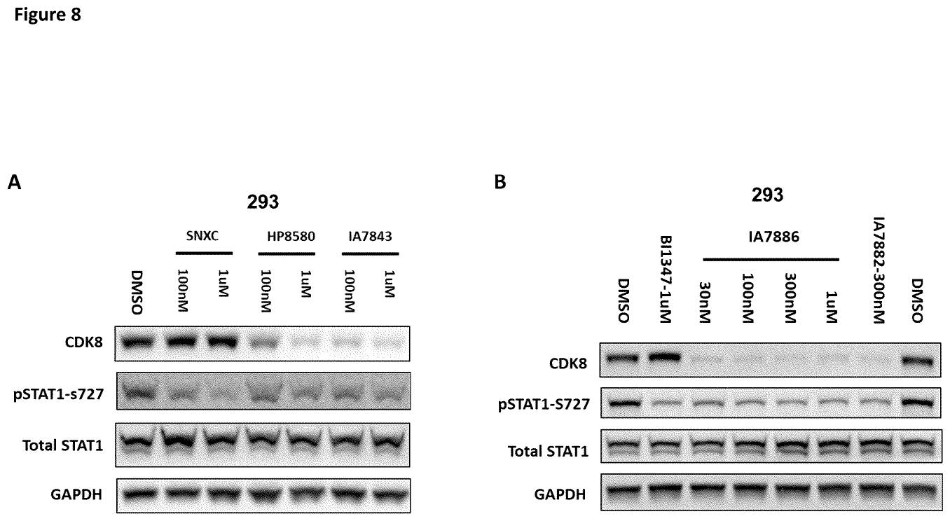

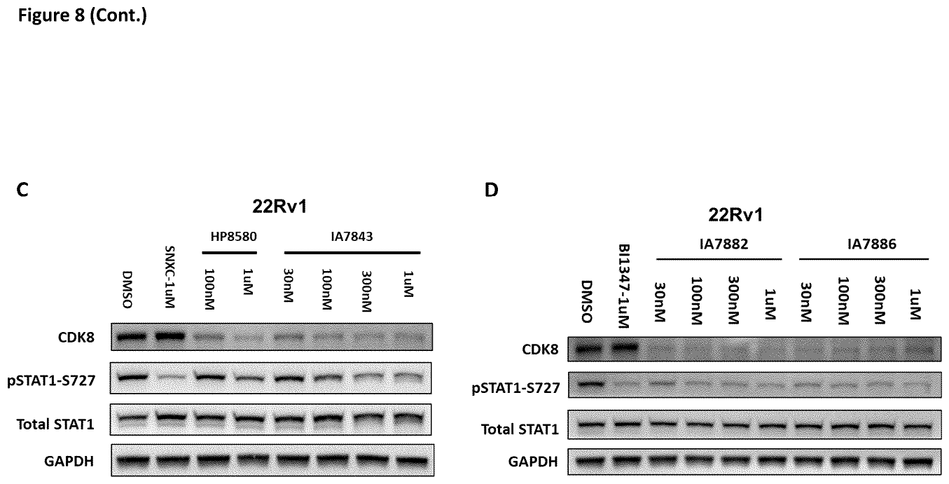

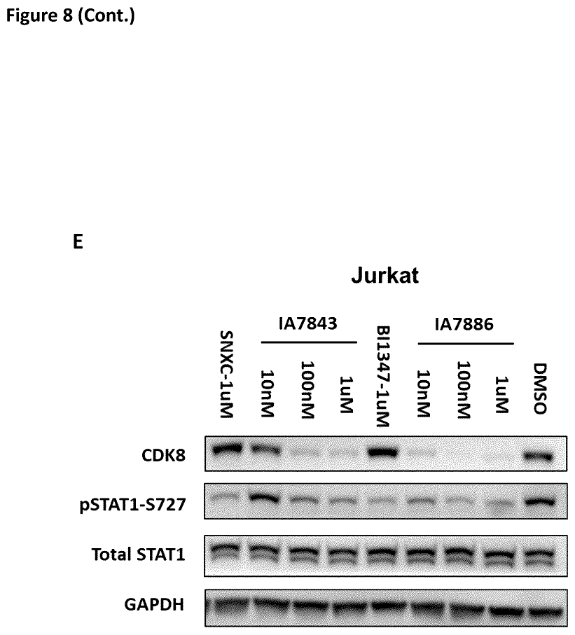

CDK8 and CDK19 are known to phosphorylate transcription factor STAT1 at S727 (although STAT1 S727 phosphorylation can also be exerted by other kinases) (Chen et al., 2019 2019). FIG. 8 show the effects of 24-hr treatment with different concentrations of HP8580, IA7843, IA7882 and IA7886 on CDK8 degradation and STAT1 S727 phosphorylation in 293, 22Rv1 and Jurkat cells. As expected, CDK8 degradation by all PROTACs was associated with decreased STAT1 S727 phosphorylation.

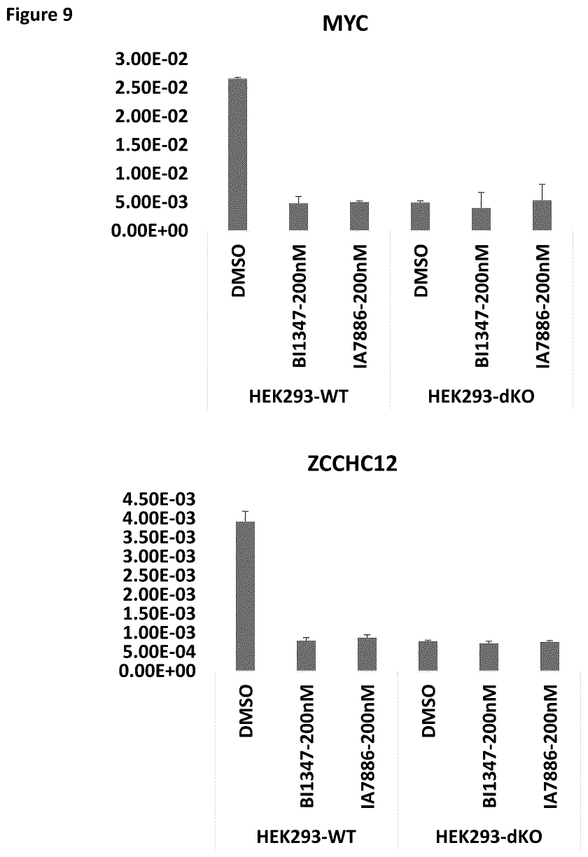

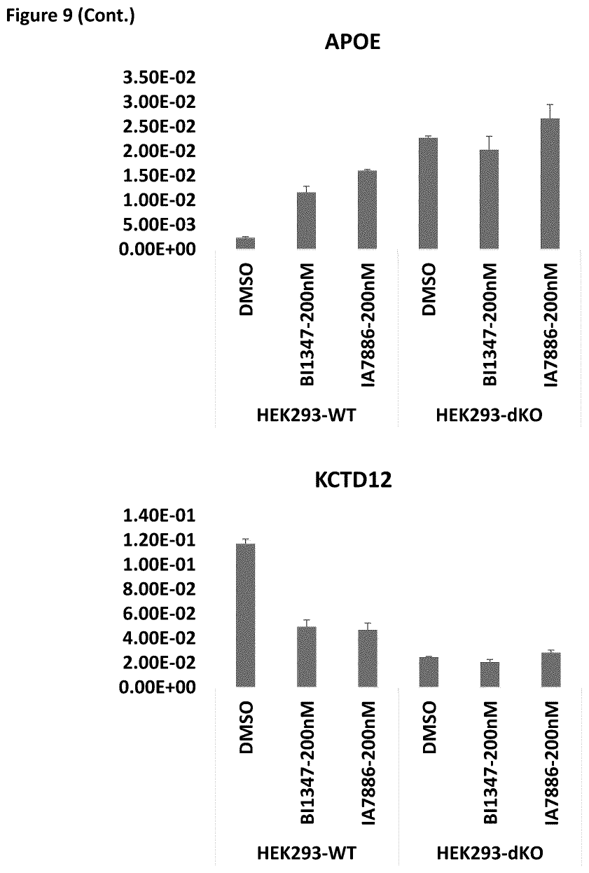

CDK8/19 act as transcriptional regulators, and therefore we tested the effects of 3-day treatment with 200 nM PROTAC IA7886 along with 200 nM CDK8/19 kinase inhibitor BI-1347 on a series of CDK8/19-regulated genes, using QPCR. As shown in FIG. 9, both IA7886 and BI-1347 had similar effects on the expression of the tested genes in wild-type HEK-293 cells; dKO showed a similar effect to PROTACs, and both IA7886 and BI-1347 had no effect in dKO cells, confirming their target selectivity. Hence, CDK8/19 PROTACs reproduce transcriptional effects of CDK8/19 kinase inhibitors and CDK8/19 knockout.

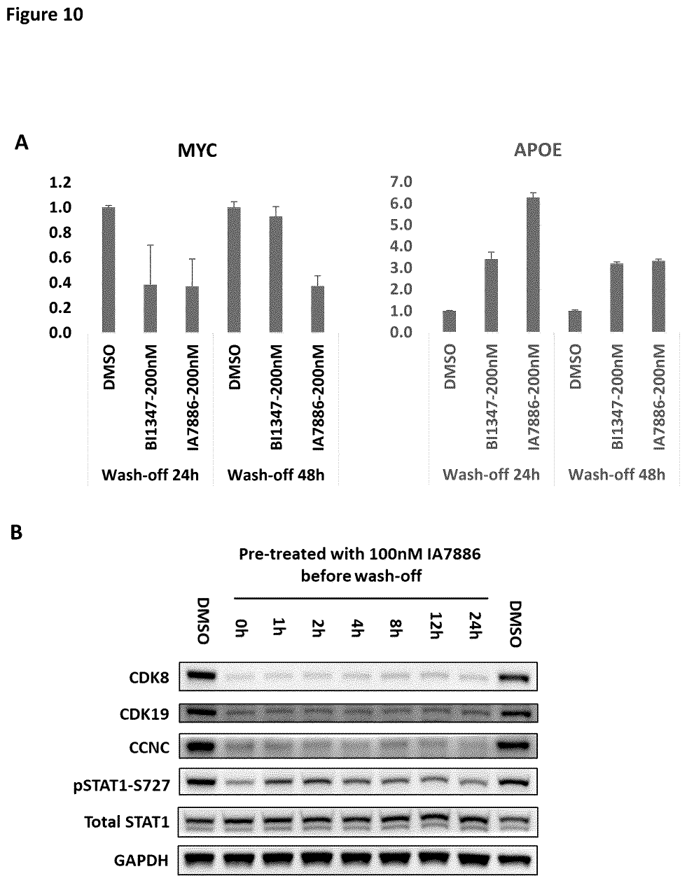

We have also compared the stability of transcriptional changes induced by a PROTAC and by a kinase inhibitor. In the experiment shown in FIG. 10A, HEK-293 cells were treated with 200 nM of BI-1347 or IA7886 for 3 days, and then the inhibitors were washed off and the restoration of transcriptional changes was monitored 24 hrs and 48 hrs after wash-off. The effects of BI-1347 were largely maintained 24 hrs after wash-off but mostly disappeared 48 hrs after wash-off. In contrast, the transcriptional effects of the PROTAC were maintained even 48 hrs after wash-off. This result indicates that CDK8/19 PROTACs show a more sustained transcriptional effect relative to CDK8/19 kinase inhibitors. In concordance with this observation, WB analysis showed that the effects of IA7886 on CDK8/CDK19/CCNC protein degradation and STAT1 S727 phosphorylation remained nearly unchanged in HEK-293 cells within 24 hours after removal of drug-containing media (FIG. 10B).

### Example 4. Effects of CDK8/19-Degrading PROTACs on Leukemia Cell Growth

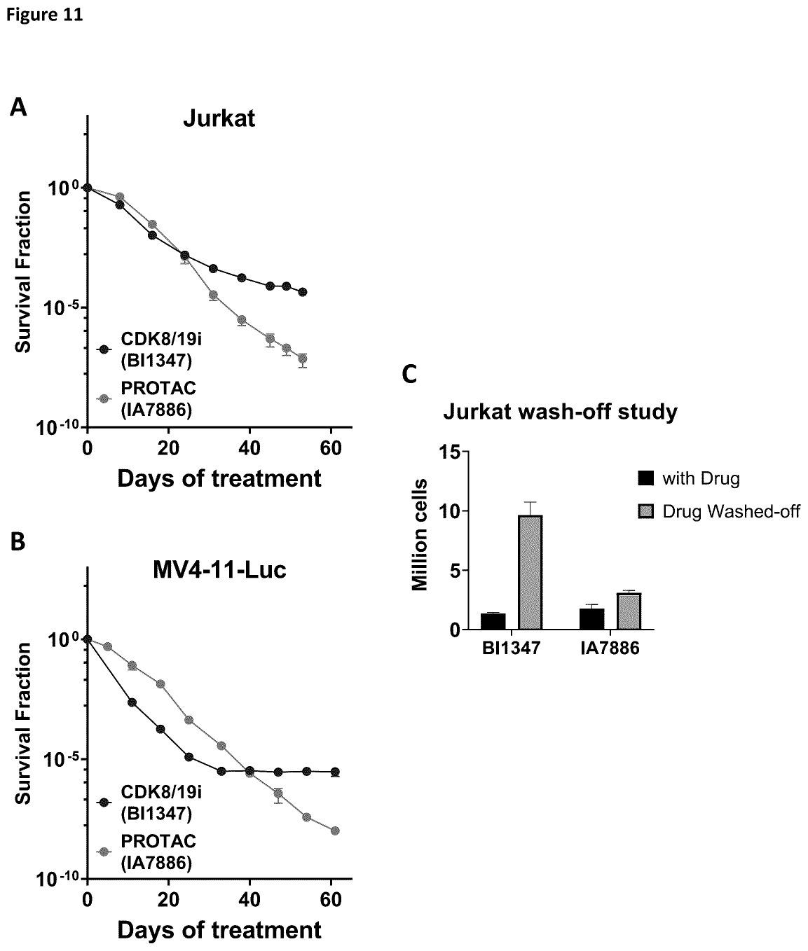

The anti-proliferative effects of a PROTAC and a kinase inhibitor were compared in leukemia cell lines. In the long-term treatment experiment shown in FIG. 11A, Jurkat cells (0.1 million cells) were continuously treated with vehicle control (0.1% DMSO), 300 nM BI-1347 or 300 nM IA7886 for 53 days (each condition in biological triplicates). Every 7-8 days, live cells were counted using BioRAD TC20 Automated Cell Counter with trypan blue staining: 0.1 million live cells were passaged to a new T25 flask to propagate under the same treatment conditions. The survival fraction was calculated by dividing the accumulated number of drug-treated cells with the accumulated number of vehicle-treated cells. The kinase inhibitor BI-1347 had a stronger growth inhibition effect than the PROTAC IA7886 at first two passages (14 days). However, in later passages, the growth rates of BI-1347 treated cells were increasing, indicating the development of resistance, while the growth rates of IA-7886 treated cells were decreasing, indicating that PROTAC can provide a more sustained growth inhibition of CDK8/19-dependent leukemia cells. This sustained inhibition property of PROTAC was also observed in MV4-11, a leukemia cell line highly sensitive to CDK8/19 kinase inhibitors. As shown in FIG. 11B, BI-1347 strongly inhibited MV4-11 cells for the first 4 passages (33 days) but after that the cells became fully resistant to the kinase inhibitor, as indicated by unchanged survival fraction up to the end of the study (60 days). On the other hand, the growth inhibition by IA7886 was much more stable and continued for the entire 60 days.

We also compared the stability of short-term growth inhibition induced by a PROTAC and a kinase inhibitor in a wash-off study in Jurkat cells. In the experiment shown in FIG. 11C, Jurkat cells were pre-treated with 200 nM BI-1347 or IA7886 for 8 days before removal of drug-containing media and further cultured in either drug-containing (200 nM) or drug-free media for 8 days before counting cell numbers. Removal of BI-1347 led to 7.1 fold increase of the cell number while removal of IA7886 increased the cell number only 1.8 fold (FIG. 11C).

Hence CDK8/19-degrading PROTACs can be more beneficial than CDK8/19 kinase inhibitors in treating leukemia.

### Example 5. Effects of CDK8/19-Degrading PROTACs on Multiple Myeloma Cell Growth

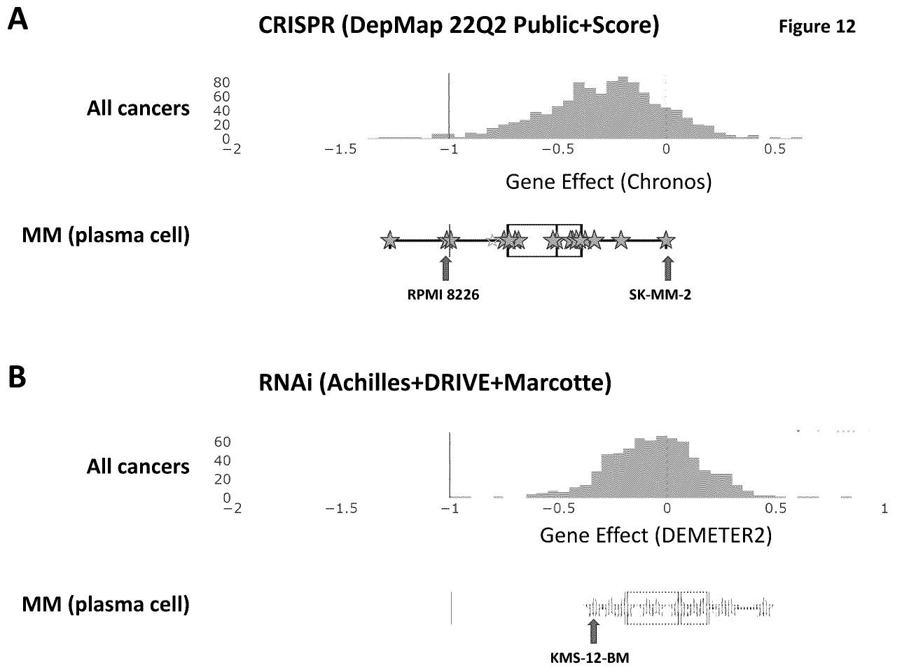

CRBN is a validated therapeutic target in Multiple Myeloma (MM), suggesting that CRBN-binding PROTACs may be especially valuable in this disease (Barankiewicz et al., 2022). Furthermore, analysis of the DepMap CRISPR database (https://depmap.org/portal/) indicates that MM cell lines are on average more sensitive than most tumor cell lines (˜1,000 cell lines of different tumor types) to CRISPR knockout of CCNC, as indicated by the negative gene effect (Chronos score) (FIG. 12A). In contrast to CRISPR data, analysis of DepMap RNAi database reveals only modest effects of partial depletion of CCNC by RNAi knockdown across different tumor cell lines, including MM, but some MM cell lines are inhibited even by RNAi knockout of CCNC, as indicated by the negative gene effect (DEMETER2 score) (FIG. 12B). Since CCNC is depleted upon treatment with CDK8/19 PROTACs but not CDK8/19 kinase inhibitors, we decided to test if MM cell lines would be preferentially responsive to CDK8/19 PROTACs.

For this analysis, we chose MM cell line RPMI 8226 (from ATCC) that showed a strong effect of CCNC CRISPR knockout (FIG. 12A) and MM cell line KMS-12-BM (from DSMZ) that showed the strongest effect of CCNC RNAi knockdown (FIG. 12B). For comparison with these CCNC-dependent cell lines, we also tested cell line SK-MM-2 (from DSMZ), the only MM cell line that showed no CCNC dependence according to CRISPR analysis (FIG. 12A). The following CDK8/19 kinase inhibitors were tested for their effects on MM cell lines over 20 days continuous treatment: CDK8/19 kinase inhibitors BI1347 (200 nM) and Senexin C (1 μM), BI1347-derived PROTAC IA7886 (200 nM) and Senexin C-derived PROTAC IA7843 (1 μM). 1-4 million cells were treated with vehicle control (0.1% DMSO), or CDK8/19 kinase inhibitors or PROTACs for 20 days. Every 4 days, cells were counted using BioRAD TC20 Automated Cell Counter with trypan blue staining, and the same number of cells were passaged to a new T25 flask to propagate under the same treatment conditions.

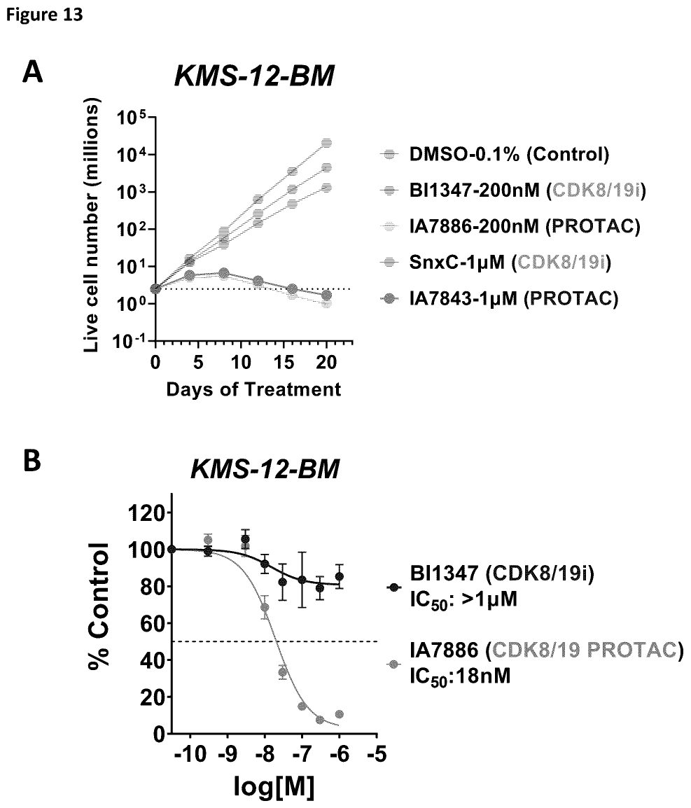

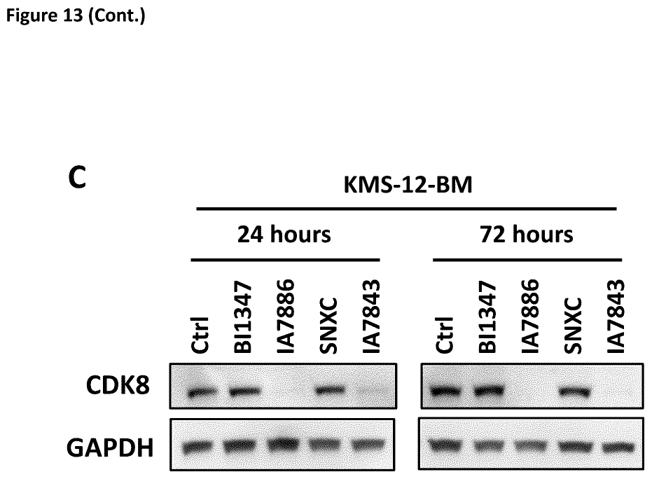

Both CDK8/19 kinase inhibitors only weakly inhibited the growth of KMS-12-BM cell line over 20 days, whereas both PROTACs inhibited cell growth for the first 8 days, and afterwards continuously decreased KMS-12-BM cell number until the end of the experiment, indicating the induction of cell death (FIG. 13A). Concentration dependence of KMS-12-BM growth inhibition over 4 days was determined for BI-1347 CDK8/19 kinase inhibitor and IA7886 PROTAC using CCK-8 assay (FIG. 13B). The kinase inhibitor had very little growth-inhibitory effect (IC50>1 HM) but the PROTAC was very efficient (IC50=18 nM). As shown in FIG. 13C, both PROTACs (but not the kinase inhibitors) efficiently degraded CDK8 in KMS-12-BM cells at the concentrations used in FIG. 13A.

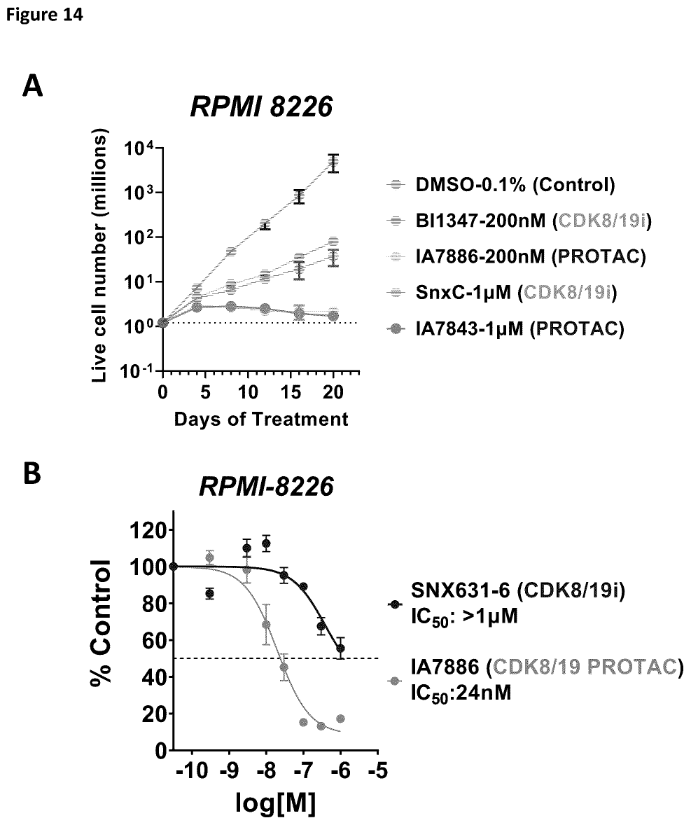

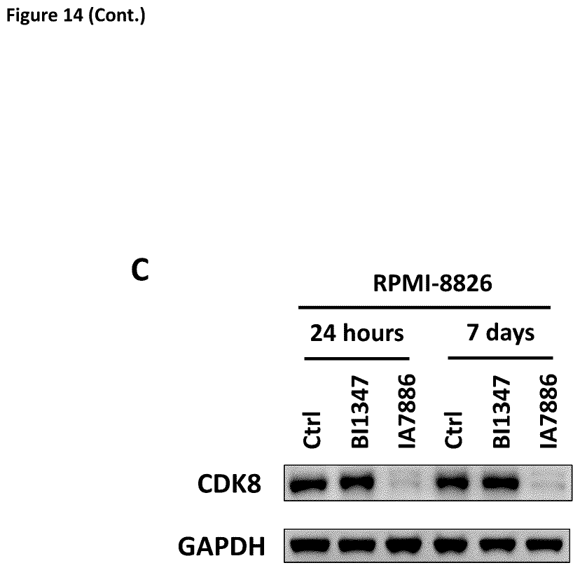

Similarly, both PROTACs strongly inhibited the growth of RPMI 8226 cells, with a decrease in cell number observed after the first 8 days (FIG. 14A). Both CDK8/19 kinase inhibitors suppressed cell growth of RPMI 8226 to a greater extent than in KMS-12-BM cells but much less efficiently than the PROTACs (FIG. 14A). Concentration dependence analysis of 4-day growth inhibition showed that the efficacy of IA7886 in RPMI 8226 cells (IC50=24 nM) was similar to its efficacy in KMS-12-BM, whereas a kinase inhibitor SNX631-6 (a.k.a. 15u-D6 (PCT/US2020/033937)) inhibited RPMI-8226 cell growth but its IC50 was >1 μM. As shown in FIG. 14C, IA7886 PROTAC (but not BI-1347 kinase inhibitor) efficiently degraded CDK8 in RPMI 8226 cells at the concentrations used in FIG. 14A.

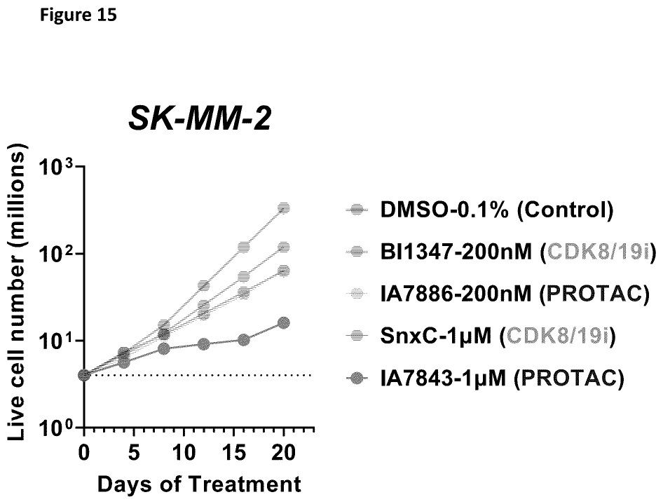

MM cell line SK-MM-2 was moderately inhibited by both CDK8/19 kinase inhibitors and PROTACs over 20 days continuous treatment, in the order of efficacy: BI1347<IA7886<Senexin C<IA7843 (FIG. 15) Although the growth-inhibitory effects of CDK8/19 PROTACs were much weaker in this CCNC-independent cell line than in the CCNC-dependent cells, growth inhibition was still apparent, indicating that CDK8/19-degrading PROTACs is therapeutically useful across a broad variety of MMs.

### Example 6. In Vivo Activity of CDK8/19-Degrading PROTACs

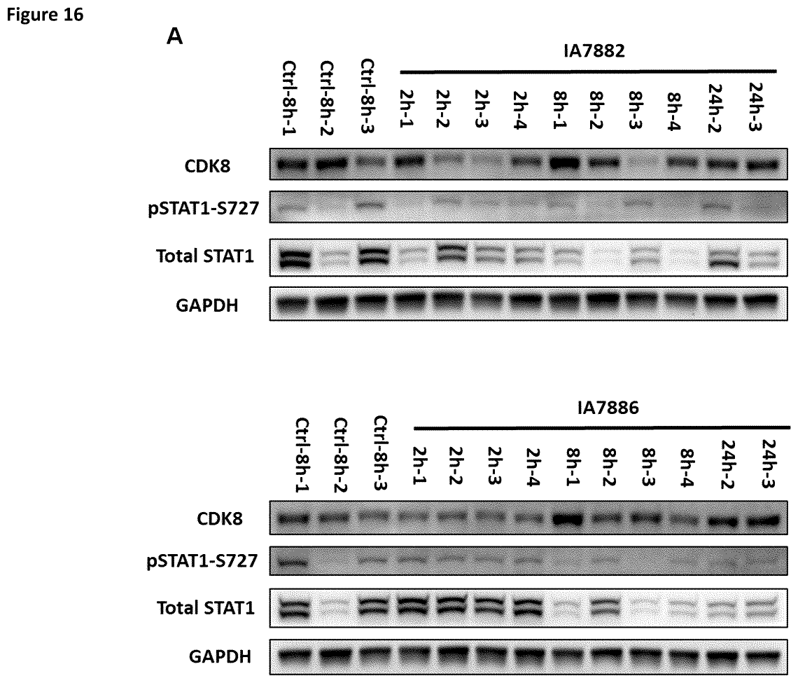

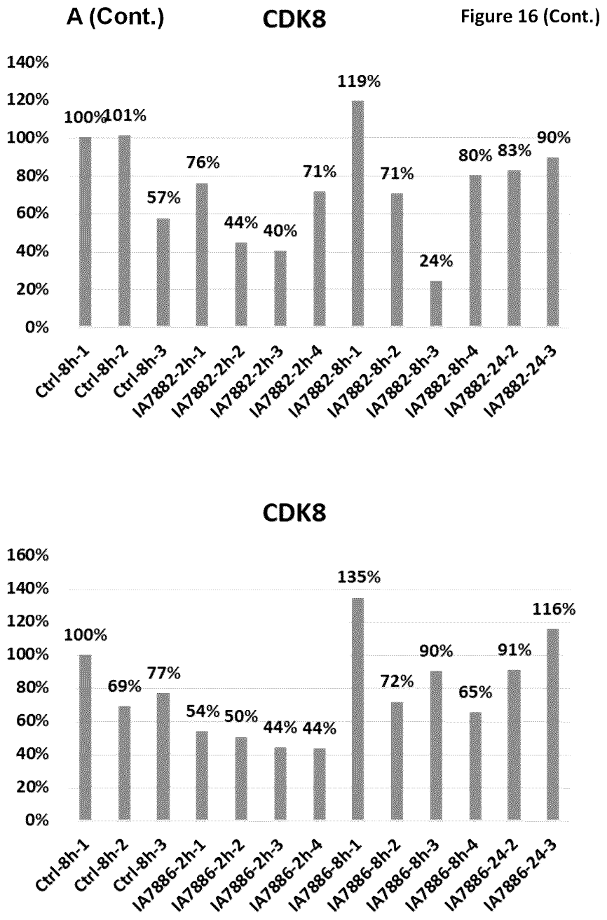

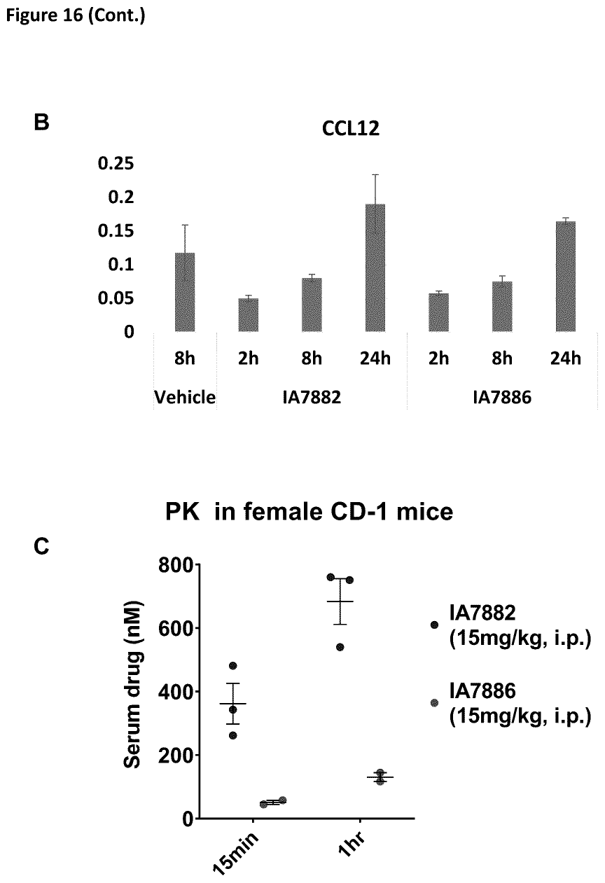

To determine if CDK8/19 targeting PROTACs can degrade CDK8 and affect CDK8-regulated gene expression in vivo, we have used a tumor model formed by CT26 murine colon carcinoma cells, where the PROTACs were found to degrade CDK8 in cell culture (FIG. 6A). CT26 cells were injected subcutaneously into syngeneic Balb/c female mice and allowed to grow to 150˜200 mm³. Tumor-bearing mice then received PROTACs IA7882 or IA7886 by oral gavage at 20 mg/kg in 30% propylene glycol/70% PEG-400 vehicle. Tumors were collected 2, 8 or 24 hrs after dosing and used to measure protein levels of CDK8, pS727 STAT1 and total STAT1 (by western blotting) and mRNA expression of CCL12, a gene that is inhibited by different CDK8/19 inhibitors in CT26 tumors in vivo (by QPCR). As shown in FIG. 12A, CDK8 levels were decreased in most of the tumor extracts collected 2 hrs or 8 hrs after dosing with IA7882 and in some of the tumors 2 hrs or 8 hrs after dosing with IA7886. Both pS727 STAT1 and total STAT1 were also decreased in most of the tumors collected 2 hrs or 8 hrs after dosing with IA7882 or IA7886. The levels of CCL12 were also decreased in tumors treated with IA7882 or IA7886 2 hrs or 8 hrs (but not 24 hrs) after dosing (FIG. 16B). Hence, these PROTACs can induce CDK8 degradation and inhibit CDK8 activity in vivo.

For preliminary evaluation of the bioavailability of the PROTACs, IA7882 and IA7886 were administered to female CD-1 mice via intraperitoneal (i.p.) injection at the dose of 15 mg/kg. Drug concentrations were measured by LCMSMS analysis of serum samples collected at 15 min and 1 hr time point. Serum drug concentrations were readily measurable and were higher for IA7882 than for IA7886 (FIG. 16C).

### Example 7. Senexin C-Based PROTACs Distinguish Between Cyclin C-Bound and Unbound CDK8

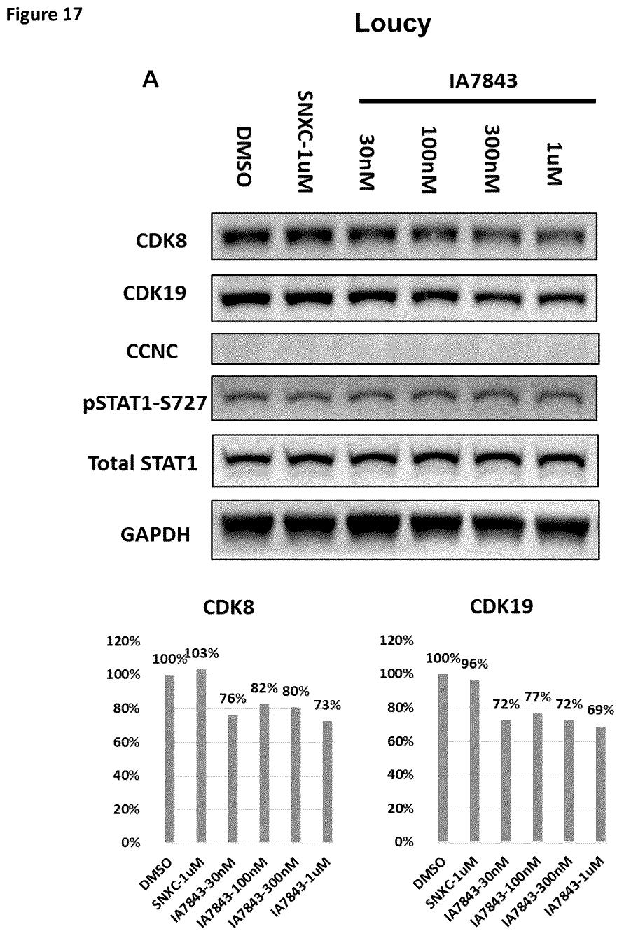

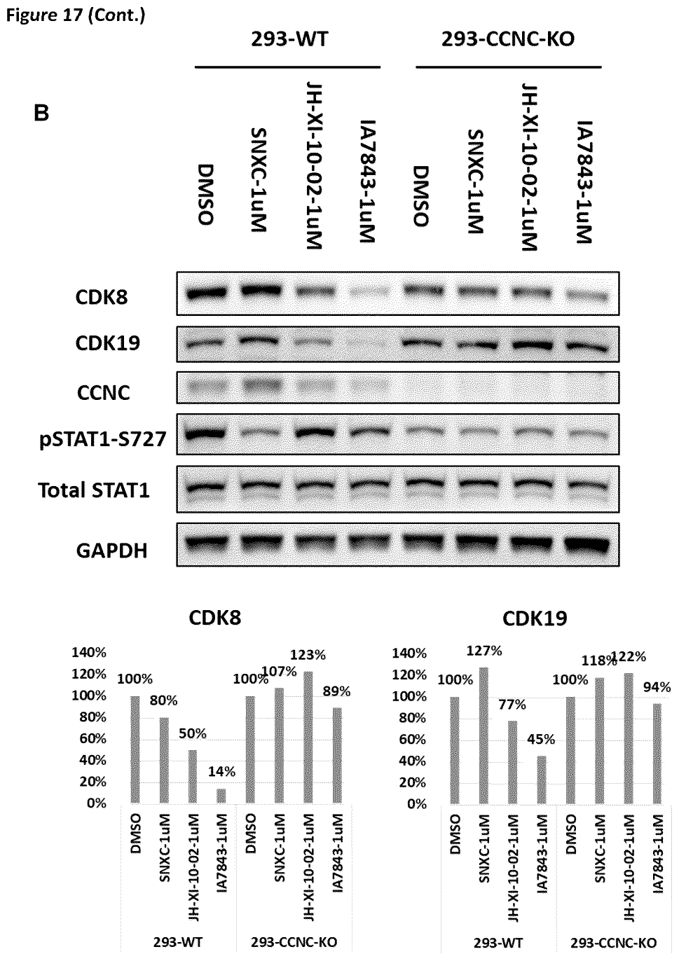

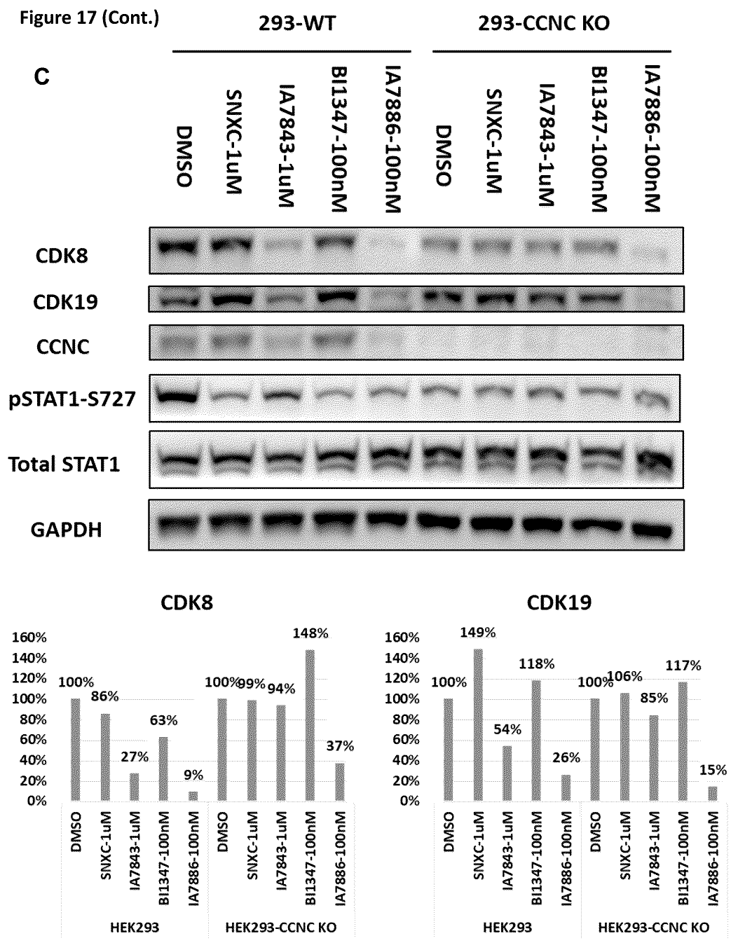

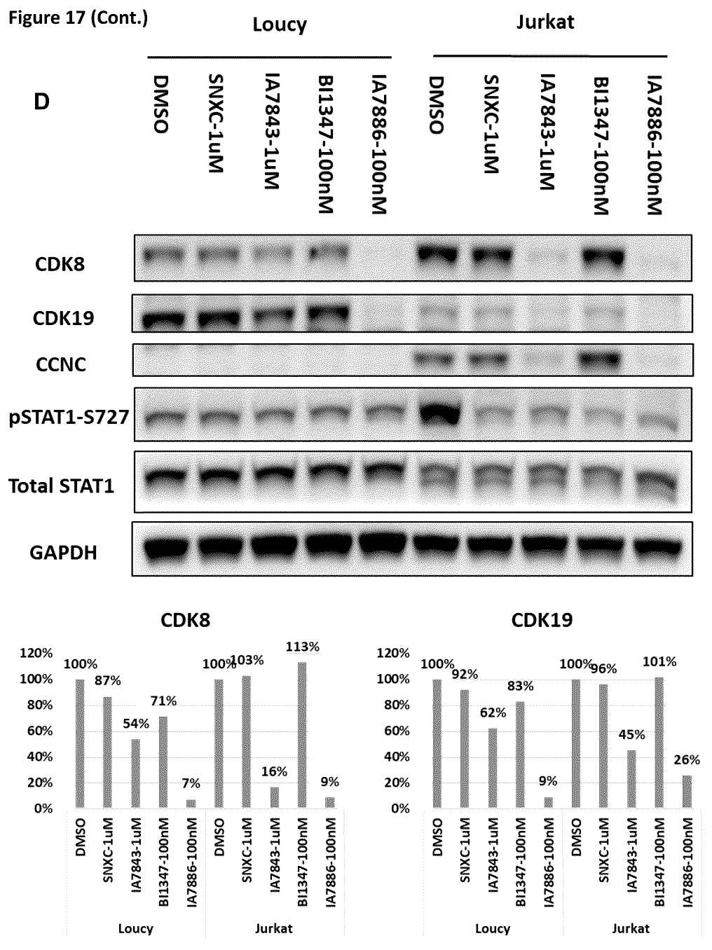

When testing the effects of Senexin C-based PROTAC IA7843 on CDK8 levels in different cell lines, we have identified one cell line, Loucy T-cell leukemia, where IA7843 induced only very weak degradation of CDK8 and CDK19 (FIG. 17A). This cell line, however, is unique in lacking Cyclin C (CCNC), the binding partner of CDK8/19. To test if the absence of CDK8-bound CCNC was responsible for the loss of IA7843 CDK8-degrading activity, we have tested the effect of IA7843 on a derivative of HEK-293cells with CRISPR-mediated knockout of CCNC. While IA7843 efficiently degraded CDK8 and CDK19 in the wild-type HEK-293cells, it was ineffective in the CCNC-deficient derivative (FIG. 17B), demonstrating that CCNC-unbound conformation of CDK8/19 is resistant to degradation by Senexin C-based PROTACs. In contrast, BI-1347-based PROTACs IA7882 and IA7886 efficiently degraded CDK8 and CDK19 in CCNC-defective 293 cells (FIG. 17C) as well as in Loucy cells (FIG. 17D). These results demonstrate that Senexin C- and BI-1347-based PROTACs can be used as chemical probes to identify the fractions of CDK8 or CDK19 that are bound or unbound to CONC.

### Synthetic Schemes

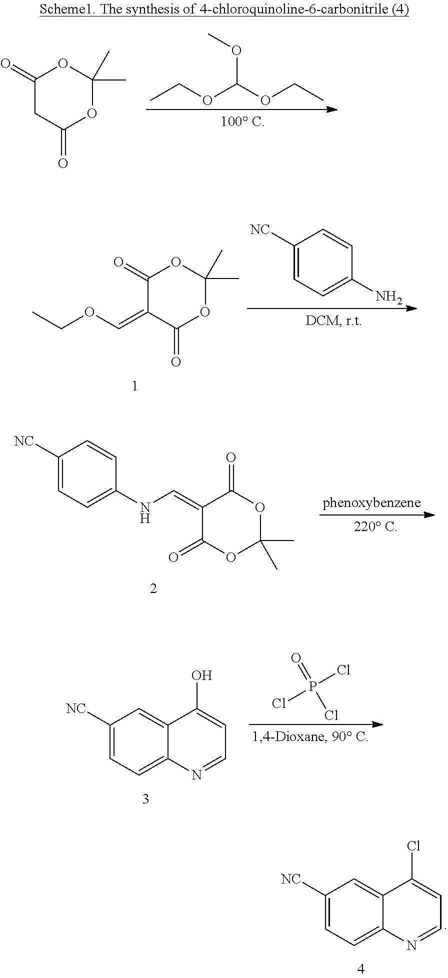

5-(ethoxymethylene)-2,2-dimethyl-1,3-dioxane-4,6-dione (1): To a 100 mL flask was added 2,2-dimethyl-1,3-dioxane-4,6-dione (34.7 mmol, 5 g), and 5-(ethoxymethylene)-2,2-dimethyl-1,3-dioxane-4,6-dione (35 mmol, 7 g). The mixture was stirred at 100° C. for 1.5 h. Upon completion, the mixture was condensed via rotavap. The resulted light-yellow solid residue was 5-(ethoxymethylene)-2,2-dimethyl-1,3-dioxane-4,6-dione (6.5 g, 94% yield) and used for the next step without further purification.

4-[(2,2-dimethyl-4,6-dioxo-1,3-dioxan-5-ylidene)methylamino]benzonitrile (2): To a 100 mL round bottom flask was charged with a solution of 4-aminobenzonitrile (17.5 mmol, 2.07 g) in DCM (80 mL). This was followed by adding 5-(ethoxymethylene)-2,2-dimethyl-1,3-dioxane-4,6-dione (32.5 mmol, 6.5 g) in DCM (20 mL). The resulting solution was at room temperature for 30 min. Upon completion, the resulting solid was filtered off and washed with hexane to give the target compound 4-[(2,2-dimethyl-4,6-dioxo-1,3-dioxan-5-ylidene)methylamino]benzonitrile as a white solid (3.7 g, 40% yield) and used without further purification. ESI-MS m/z: 273 ([M+H]⁺).

4-hydroxyquinoline-6-carbonitrile (3): To a 250 mL round bottom flask was added with 4-[(2,2-dimethyl-4,6-dioxo-1,3-dioxan-5-ylidene)methylamino]benzonitrile (13.6 mmol, 3.7 g) and phenoxybenzene (40 mL). The resulting solution was at 220° C. for 40 min. Upon completion, the resulting solution was cooled to room temperature and hexane (100 mL) was added. The precipitate was collected by filtration and washed with hexane, the crude compound was purified via flash column chromatography using a gradient of 0-5% MeOH/DCM to give 4-hydroxyquinoline-6-carbonitrile as a brown solid (1.5 g, 65% yield). ESI-MS m/z: 273 ([M+H]⁺).

4-chloroquinoline-6-carbonitrile (4): To a 100 mL round-bottom flask was placed a solution of 4-hydroxyquinoline-6-carbonitrile (5.88 mmol, 1 g) in dioxane (30 mL), and POCl₃ (29.4 mmol, 2.69 mL) was added. Reaction was stirred for 1.5 h at 90° C. Upon completion, solvent was removed under reduced pressure. The pH value of the solution was adjusted to 8 with saturated sodium carbonate solution. The resulting solution was extracted with three times (20 ml each) of ethyl acetate, organic layers were combined and dried over anhydrous sodium sulfate and concentrated in vacuum. The crude was purified using flash column chromatography using a gradient of 0-2% MeOH/DCM to give 4-chloroquinoline-6-carbonitrile as a white solid (0.48 g, 44% yield). ESI-MS m/z: 189 ([M+H]⁺). ¹H-NMR (300 MHz, CD₃OD): δ 8.94 (d, J=4.8 Hz, 1H), 8.76 (d, J=1.5 Hz, 1H), 8.24 (d, J=8.8 Hz, 2H), 8.07 (dd, J=1.8, 8.9 Hz, 1H), 7.82 (d, J=5.0 Hz, 1H).

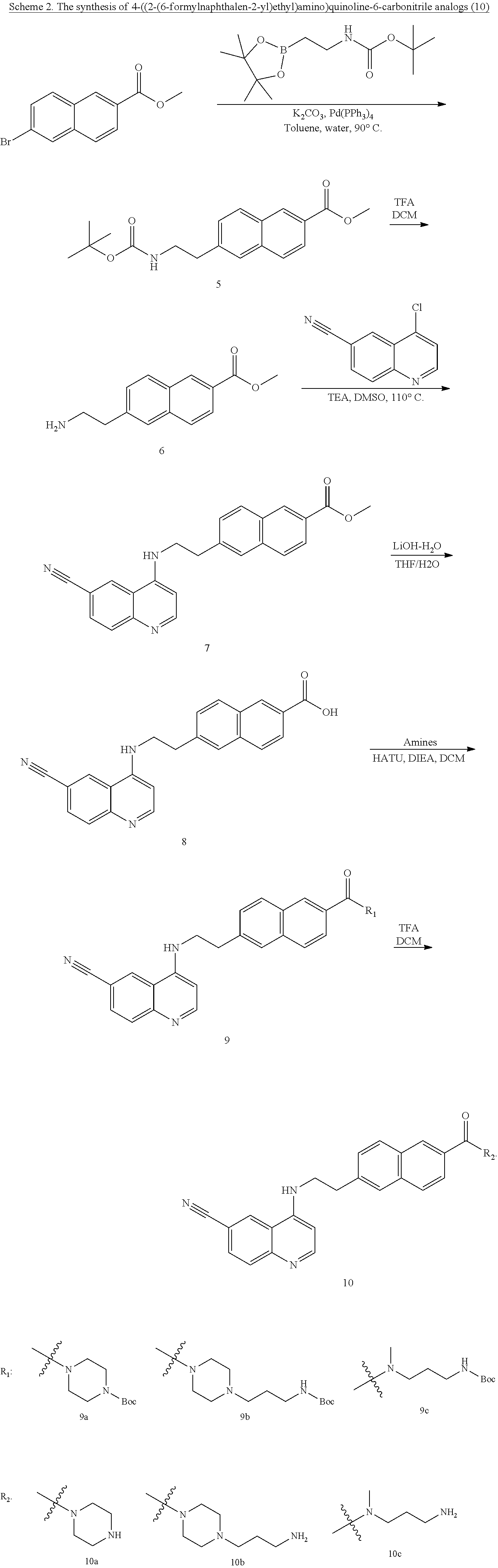

methyl 6-[2-(tert-butoxycarbonylamino)ethyl]naphthalene-2-carboxylate (5): To a 10 mL microwave tube was added a solution of methyl 6-bromonaphthalene-2-carboxylate (1.89 mmol, 500 mg), tert-butyl N-[2-(4,4,5,5-tetramethyl-1,3,2-dioxaborolan-2-yl)ethyl]carbamate (1.89 mmol, 511 mg), Pd(dppf)Cl₂ thiourea (2.62 mmol, 199 mg) and 4M HCl in 1,4-dioxane (1 drop) in EtOH (4 mL). The mixture was (0.19 mmol, 138 mg), Cs₂CO₃ (3.77 mmol, 1.23 g), Ag₂O (2.83 mmol, 656 mg), and water (3.77 mmol, 68 mg) in THF (7 mL). The reaction was microwaved at 85° C. for 2 h. Upon completion, the mixture was diluted with ethyl acetate, added with water, extracted with ethyl acetate, the organic layers were combined, dried by Na₂SO₄, condensed and purified by flash column using a gradient of 0-40% ethyl acetate/hexane to give methyl 6-[2-(tert-butoxycarbonylamino)ethyl]naphthalene-2-carboxylate (451 mg, 73% yield). ESI-MS m/z: 330 ([M+H]⁺). ¹H-NMR (300 MHz, CD₂Cl₂): 8.57 (s, 1H), 8.02 (d, J=8.5 Hz, 1H), 7.92 (d, J=8.5 Hz, 1H), 7.84 (d, J=8.5 Hz, 1H), 7.70 (s, 1H), 7.43 (d, J=8.5 Hz, 1H), 4.67 (m, 1H), 3.95 (s, 3H), 3.45 (q, J=6.7 Hz, 2H), 2.97 (t, J=6.7 Hz, 2H), 1.39 (s, 9H).

methyl 6-(2-aminoethyl) naphthalene-2-carboxylate (6): To a 100 mL round-bottom flask was added a solution of methyl 6-[2-(tert-butoxycarbonylamino)ethyl]naphthalene-2-carboxylate (1.37 mmol, 451 mg) in DCM (5 mL). Then TFA (1 mL) was added and the mixture was stirred at room temperature for 4 h. Upon completion, the mixture was condensed to give methyl 6-(2-aminoethyl) naphthalene-2-carboxylate (314 mg, 100% yield) and used for the next step without further purification. ESI-MS m/z: 230 ([M+H]⁺).

methyl 6-[2-[(6-cyano-4-quinolyl)amino]ethyl]naphthalene-2-carboxylate (7): To a 100 mL round-bottle flask was added a solution of methyl 6-(2-aminoethyl) naphthalene-2-carboxylate (5.67 mmol, 1.3 g), 4-chloroquinoline-6-carbonitrile (5.67 mmol, 1.07 g) and DIEA (11.3 mmol, 1.47 g) in DMSO (10 mL). The mixture was then heated to 110° C. for overnight. After cooling to room temperature, the mixture was added with water, extracted with DCM, the organic layers were combined, dried over anhydrous MgSO₄ and concentrated under reduced pressure. The residue was purified by flash column using a gradient of 0-8% MeOH/DCM to give methyl 6-[2-[(6-cyano-4-quinolyl)amino]ethyl]naphthalene-2-carboxylate (1.21 g, 56% yield). ESI-MS m/z: 382 ([M+H]⁺). ¹H-NMR (300 MHz, CD₂Cl₂): 8.59 (d, J=5.4 Hz, 1H), 8.58 (s, 1H), 8.28 (s, 1H), 8.03 (d, J=8.9 Hz, 1H), 7.95 (d, J=8.9 Hz, 2H), 7.84 (d, J=8.9 Hz, 1H), 7.77 (s, 1H), 7.72 (d, J=8.9 Hz, 1H), 7.48 (d, J=8.9 Hz, 1H), 6.59 (d, J=5.4 Hz, 1H), 5.93 (m, 1H), 3.94 (s, 3H), 3.72 (q, J=6.7 Hz, 2H), 3.25 (t, J=6.7 Hz, 2H).

6-[2-[(6-cyano-4-quinolyl)amino]ethyl]naphthalene-2-carboxylic acid (8): To a 10 mL round-bottle flask was added a solution of methyl 6-[2-[(6-cyano-4-quinolyl)amino]ethyl]naphthalene-2-carboxylate (0.16 mmol, 60 mg) and LiOH—H₂O (0.32 mmol, 14 mg) in THF (2 mL) and water (1 mL). The mixture was stirred at 60° C. for overnight. Upon completion, the mixture was condensed to give 6-[2-[(6-cyano-4-quinolyl)amino]ethyl]naphthalene-2-carboxylic acid (57 mg, 99% yield) and used for the next step without further purification. ESI-MS m/z: 368 ([M+H]⁺).

General method for 9a-c: To a round-bottom flask was added a solution of 6-(cyanomethyl) naphthalene-2-carboxylic acid (1 eq) in DMF (5 mL), then HATU (1.5 eq), DIPEA (3 eq) were added and stirred for 15 min. After that, piperazine derivatives (1 eq) was added and the mixture was stirred for 2 h. Upon completion, the mixture was diluted with water, extracted with DCM, the organic layers were combined, dried by Na₂SO₄, condensed and purified by flash column chromatography using a gradient of 0-5% MeOH/DCM to give the target compound.

tert-butyl 4-[6-[2-[(6-cyano-4-quinolyl)amino]ethyl]naphthalene-2-carbonyl]piperazine-1-carboxylate (9a): A light yellow solid (179 mg, yield 43%). ESI-MS m/z: 536 ([M+H]⁺). ¹H-NMR (300 MHz, DMSO-d6): 8.90 (s, 1H), 8.53 (d, J=5.9 Hz, 1H), 8.05 (m, 1H), 7.97-7.88 (m, 6H), 7.58 (dd, J=8.4, 1.3 Hz, 1H), 7.49 (dd, J=8.4, 1.3 Hz, 1H), 6.79 (d, J=5.9 Hz, 1H), 3.71 (m, 2H), 3.58 (m, 4H), 3.47 (m, 2H), 3.40 (m, 2H), 3.19 (m, 2H), 1.41 (s, 9H).

tert-butyl N-[3-[4-[6-[2-[(6-cyano-4-quinolyl)amino]ethyl]naphthalene-2-carbonyl]piperazin-1-yl]propyl]carbamate (9b): A light yellow solid (191 mg, 46% yield). ESI-MS m/z: 593 ([M+H]⁺). ¹H-NMR (300 MHz, DMSO-d6): 9.66 (s, 1H), 9.10 (s, 1H), 8.61 (d, J=7.3 Hz, 1H), 8.27 (dd, J=8.8, 1.3 Hz, 1H), 8.03 (d, J=8.8 Hz, 1H), 7.95 (d, J=8.5 Hz, 2H), 7.89 (s, 1H), 7.85 (d, J=8.5 Hz, 2H), 7.59 (dd, J=8.8, 1.3 Hz, 1H), 7.50 (m, 1H), 7.10 (d, J=7.3 Hz, 1H), 3.94 (q. J=6.9 Hz, 2H), 3.57 (m, 2H), 3.22 (t, J=7.3 Hz, 2H), 2.95 (s, 3H), 2.92 (m, 2H), 1.91 (m, 2H).

tert-butyl N-[3-[[6-[2-[(6-cyano-4-quinolyl)amino]ethyl]naphthalene-2-carbonyl]-methyl-amino]propyl]carbamate (9c): A white solid (360 mg, 59% yield). ESI-MS m/z: 538 ([M+H]⁺). ¹H-NMR (300 MHz, CD₂Cl₂). 8.55 (d, J=5.1 Hz, 1H), 8.22 (s, 1H), 7.94 (d, J=8.6 Hz, 1H), 7.83 (d, J=8.6 Hz, 2H), 7.79 (s, 1H), 7.72 (dd, J=8.6, 2.0 Hz, 1H), 7.67 (s, 1H), 7.47 (d, J=8.5 Hz, 1H), 7.41 (d, J=8.5 Hz, 1H), 6.59 (d, J=5.1 Hz, 1H), 5.83 (m, 1H), 5.47 (m, 1H), 3.66 (m, 4H), 3.20 (m, 4H), 2.94 (s, 3H), 1.82 (m, 2H), 1.43 (s, 9H).

General method for 10a-c: To a 10 mL round-bottom flask was added a solution of 9a-c in DCM (5 mL). Then TFA (1 mL) was added and the mixture was stirred at room temperature for 4 h. Upon completion, the mixture was condensed to give target compounds and used for the next step without further purification.

4-[2-[6-(piperazine-1-carbonyl)-2-naphthyl]ethylamino]quinoline-6-carbonitrile (10a): A yellow solid (610 mg, 99% yield) and used for the next step without further purification. ESI-MS m/z: 436 ([M+H]⁺).

4-[2-[6-[4-(3-aminopropyl) piperazine-1-carbonyl]-2-naphthyl]ethylamino]quinoline-6-carbonitrile (10b): A yellow solid (185 mg, 95% yield) and used for the next step without further purification. ESI-MS m/z: 607 ([M+H]⁺). ¹H-NMR (300 MHz, DMSO-d6): 9.66 (s, 1H), 9.11 (s, 1H), 8.63 (d, J=7.4 Hz, 1H), 8.28 (d, J=8.7 Hz, 1H), 8.04 (s, 1H), 8.03 (d, J=7.4 Hz, 1H), 7.94 (m, 4H), 7.62 (d, J=8.7 Hz, 2H), 7.56 (d, J=8.7 Hz, 1H), 7.11 (d, J=7.4 Hz, 1H), 3.94 (q, J=6.7 Hz, 2H), 3.45 (m, 2H), 3.21 (m, 8H), 2.88 (m, 2H), 1.96 (m, 2H), 1.24 (m, 2H).

N-(3-aminopropyl)-6-[2-[(6-cyano-4-quinolyl)amino]ethyl]-N-methyl-naphthalene-2-carboxamide (10c): A yellow solid (280 mg, 96% yield) and used for the next step without further purification. ESI-MS m/z: 438 ([M+H]⁺).

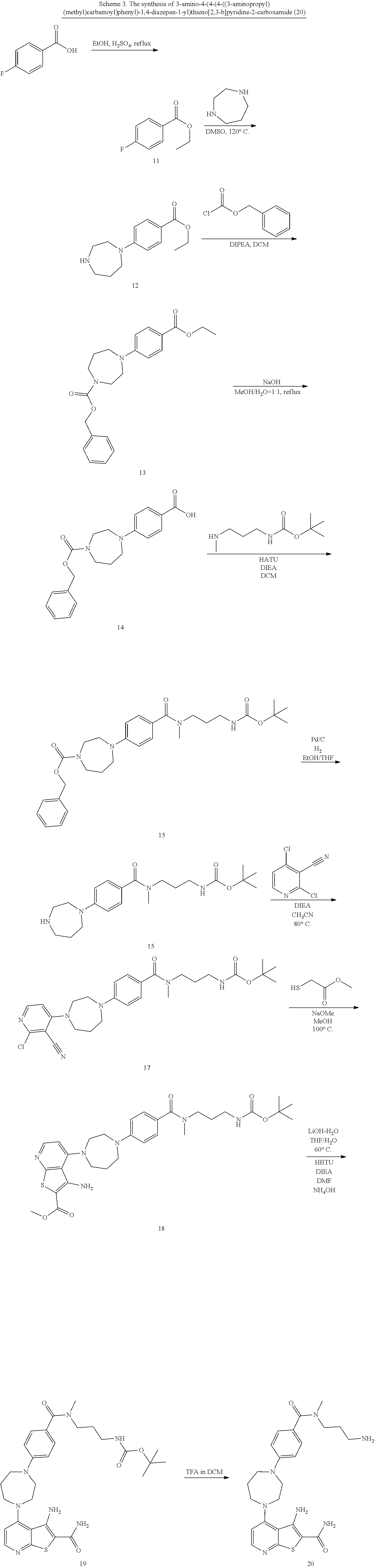

ethyl 4-fluorobenzoate (11): To a 100 mL round-bottom flask was added a solution of ethyl 4-fluorobenzoic acid (35.7 mmol, 5 g) in EtOH (20 mL). Then conc. H₂SO₄ (35.7 mmol, 3.5 g) was added and the mixture was reflux for overnight. Upon completion the mixture was cooled to r.t. and condensed and ice water was added, the mixture was neutralized by Na₂CO₃ to pH=8, the extracted with DCM for three times and the organic layers were combined and dried by Na₂SO₄ and condensed to give ethyl 4-fluorobenzoate (4 g, yield 67%) and used without further purification ESI-MS m/z: 169 ([M+H]⁺).

ethyl 4-(1,4-diazepan-1-yl) benzoate (12): To a 100 mL round-bottom flask was added a solution of ethyl 4-fluorobenzoate (11.9 mmol, 2 g) and 1,4-diazepane (23.8 mmol, 2.38 g) in DMSO (15 mL). Then the mixture was stirred at 120° C. for overnight. Upon completion, the mixture was cooled to room temperature, diluted with water, extracted with DCM, the organic layers were combined, dried by Na₂SO₄, condensed and purified by flash column chromatography using a gradient of 0-8% MeOH/DCM to give ethyl 4-(1,4-diazepan-1-yl) benzoate (2.46 g, yield 83%). ESI-MS m/z: 249 ([M+H]⁺).

benzyl 4-(4-(ethoxycarbonyl)phenyl)-1,4-diazepane-1-carboxylate (13): To a 100 ml round-bottom flask was added a solution of ethyl 4-(1,4-diazepan-1-yl) benzoate (6.0 mmol, 1.5 g) and benzyl carbonochloridate (6.0 mmol, 1.02 g) in DCM (10 mL), then then DIEA (12.0 mmol, 1.64 g) was added and the mixture was stirred at room temperature for overnight. After that, the mixture was added with water, extracted with DCM, the organic layers were combined, washed with brine, dried by Na₂SO₄, condensed to give benzyl 4-(4-(ethoxycarbonyl)phenyl)-1,4-diazepane-1-carboxylate (2.4 g, 96%). ESI-MS m/z: 383 ([M+H]⁺).

4-[(4-benzyloxycarbonyl-1,4-diazepan-1-yl)methyl]benzoic acid (14): To a 100 mL round-bottom flask was added a solution of benzyl 4-[(4-methoxycarbonylphenyl)methyl]-1,4-diazepane-1-carboxylate (7.53 mmol, 2.88 g) in EtOH (10 mL) and water (10 mL). Then NaOH (15.1 mmol, 602 mg) was added and the mixture was refluxed for 4 h. Upon completion, the mixture was cooled to room temperature, condensed, diluted with water, acidified by 2N HCl to pH=4, extracted with DCM, the organic layers were combined, dried by Na₂SO₄, condensed to give 4-[(4-benzyloxycarbonyl-1,4-diazepan-1-yl)methyl]benzoic acid (2.77 g. yield 99%). ESI-MS m/z: 369 ([M+H]⁺).

benzyl 4-[4-[3-(tert-butoxycarbonylamino)propyl-methyl-carbamoyl]phenyl]-1,4-diazepane-1-carboxylate (15): To a 100 mL round-bottom flask was added a solution of 4-(4-benzyloxycarbonyl-1,4-diazepan-1-yl) benzoic acid (2.82 mmol, 1 g) in DCM (30 mL). Then HATU (4.23 mmol, 1.61 g), DIPEA (5.64 mmol, 729 mg) were added and stirred for 15 min. After that, tert-butyl N-[3-(methylamino)propyl]carbamate (3.4 mmol, 637 mg) was added and the mixture was stirred for 2 h. Upon completion, the mixture was diluted with water, extracted with DCM, the organic layers were combined, dried by Na₂SO₄, condensed and purified by flash column chromatography using a gradient of 0-7% MeOH/DCM to give benzyl 4-[4-[3-(tert-butoxycarbonylamino)propyl-methyl-carbamoyl]phenyl]-1,4-diazepane-1-carboxylate (1.01 g, 68%). ESI-MS m/z: 525 ([M+H]⁺). ¹H-NMR (300 MHz, CDCl₃): 7.33 (d, J=13.6 Hz, 2H), 7.32 (m, 5H), 6.65 (m, 2H), 5.11 (d, J=13.6 Hz, 2H), 3.63 (m, 2H), 3.56 (m, 2H), 3.34 (m, 2H), 3.14 (m, 2H), 3.03 (s, 6H), 1.98 (m, 2H), 1.77 (m, 2H), 1.42 (s, 9H).

tert-butyl N-[3-[[4-(1,4-diazepan-1-yl) benzoyl]-methyl-amino]propyl]carbamate (16): To a 100 mL round-bottom flask was added a solution of benzyl 4-[4-[3-(tert-butoxycarbonylamino)propyl-methyl-carbamoyl]phenyl]-1,4-diazepane-1-carboxylate (1.93 mmol, 1 g) in THF (5 mL) and EtOH (5 mL). Then Pd/C (1.93 mmol, 205 mg) was added and the mixture was saturated with H₂, stirred for overnight. After that, the mixture was diluted with hexane, filtered and the residue was washed with hexane, the filtration was condensed to give tert-butyl N-[3-[[4-(1,4-diazepan-1-yl) benzoyl]-methyl-amino]propyl]carbamate (634 mg, 84%). ESI-MS m/z: 391 ([M+H]⁺).

tert-butyl N-[3-[[4-[4-(3-chloro-2-cyano-4-pyridyl)-1,4-diazepan-1-yl]benzoyl]-methyl-amino]propyl]carbamate (17): To a 100 mL round-bottom flask was added a solution of tert-butyl N-[3-[[4-(1,4-diazepan-1-yl) benzoyl]-methyl-amino]propyl]carbamate (1.62 mmol, 634 mg) and 2,4-dichloropyridine-3-carbonitrile (1.95 mmol, 337 mg) in acetonitrile (15 mL). Then DIPEA (3.25 mmol, 420 mg) was added and the mixture was stirred at 80° C. for overnight. After that, the mixture was cooled to room temperature, added with water, extracted with DCM, the organic layers were combined, dried by Na₂SO₄, condensed and purified by flash column chromatography using a gradient of 0-7% MeOH/DCM to give tert-butyl N-[3-[[4-[4-(3-chloro-2-cyano-4-pyridyl)-1,4-diazepan-1-yl]benzoyl]-methyl-amino]propyl]carbamate (567 mg, 66%). ESI-MS m/z: 528 ([M+H]⁺). ¹H-NMR (300 MHz, CDCl₃): 8.00 (d, J=6.8 Hz, 1H), 7.33 (d, J=8.5 Hz, 2H), 6.70 (d, J=8.8 Hz, 2H), 6.60 (d, J=6.4 Hz, 1H), 4.01 (m, 2H), 3.83 (m, 2H), 3.71 (m, 2H), 3.61 (m, 2H), 3.54 (m, 2H), 3.13 (m, 2H), 3.02 (s, 6H), 2.14 (m, 2H), 1.78 (m, 2H), 1.42 (s, 9H).

methyl 3-amino-4-[4-[4-[3-(tert-butoxycarbonylamino)propyl-methyl-carbamoyl]phenyl]-1,4-diazepan-1-yl]thieno[2,3-b]pyridine-2-carboxylate (18): To a 100 mL round-bottom flask was added a solution of tert-butyl N-[3-[[4-[4-(2-chloro-3-cyano-4-pyridyl)-1,4-diazepan-1-yl]benzoyl]-methyl-amino]propyl]carbamate (1.04 mmol, 550 mg) and methyl 2-sulfanylacetate (3.13 mmol, 332 mg) in MeOH (5 mL). Then MeONa (0.42 mmol, 250 mg) was added and the mixture was stirred at 100° C. for overnight. After that, the mixture was cooled to room temperature, condensed and purified by flash column chromatography using a gradient of 0-8% MeOH/DCM to give methyl 3-amino-4-[4-[4-[3-(tert-butoxycarbonylamino)propyl-methyl-carbamoyl]phenyl]-1,4-diazepan-1-yl]thieno[2,3-b]pyridine-2-carboxylate (250 mg, 40%). ESI-MS m/z: 528 ([M+H]⁺). ¹H-NMR (300 MHz, CDCl₃): 8.48 (d, J=5.0 Hz, 1H), 7.37 (d, J=8.8 Hz, 2H), 6.92 (d, J=5.0 Hz, 1H), 6.79 (s, 2H), 6.72 (d, J=8.8 Hz, 2H), 3.86 (s, 3H), 3.80 (t, J=4.7 Hz, 2H), 3.66 (t, J=6.3 Hz, 2H), 3.56 (m, 2H), 3.38 (m, 2H), 3.27 (m, 2H), 3.14 (m, 2H), 3.06 (s, 3H), 2.21 (m, 2H), 1.79 (m, 2H), 1.43 (s, 9H).

tert-butyl N-[3-[[4-[4-(3-amino-2-carbamoyl-thieno[2,3-b]pyridin-4-yl)-1,4-diazepan-1-yl]benzoyl]-methyl-amino]propyl]carbamate (19): To a 25 mL round-bottom flask was added a solution of methyl 3-amino-4-[4-[4-[3-(tert-butoxycarbonylamino)propyl-methyl-carbamoyl]phenyl]-1,4-diazepan-1-yl]thieno[2,3-b]pyridine-2-carboxylate (0.41 mmol, 243 mg) in THF (3 mL) and water (3 mL), then LiOH—H₂O (1.22 mmol, 52 mg) was added. The mixture was stirred at 50° C. for overnight. Upon completion, the mixture was condensed and dissolved in DMF (5 mL). Then HATU (0.63 mmol, 237 mg), DIPEA (0.84 mmol, 108 mg) were added and stirred for 15 min. After that, NH₄OH (1.67 mmol, 59 mg) was added and the mixture was stirred for 2 h. Upon completion, the mixture was diluted with water, extracted with DCM, the organic layers were combined, dried by Na₂SO₄, condensed and purified by flash column chromatography using a gradient of 0-7% MeOH/DCM to give tert-butyl N-[3-[[4-[4-(3-amino-2-carbamoyl-thieno[2,3-b]pyridin-4-yl)-1,4-diazepan-1-yl]benzoyl]-methyl-amino]propyl]carbamate (149 mg, 61%). ESI-MS m/z: 582 ([M+H]⁺). ¹H-NMR (300 MHz, CDCl₃): 8.47 (d, J=5.8 Hz, 1H), 7.36 (d, J=8.9 Hz, 2H), 6.97 (s, 2H), 6.94 (d, J=5.8 Hz, 1H), 6.71 (d, J=8.9 Hz, 2H), 5.39 (s, 2H), 3.80 (t, J=5.6 Hz, 2H), 3.65 (t, J=6.7 Hz, 2H), 3.55 (m, 2H), 3.37 (m, 2H), 3.28 (m, 2H), 3.14 (m, 2H), 3.05 (s, 3H), 2.20 (m, 2H), 1.79 (m, 2H), 1.43 (s, 9H).

3-amino-4-[4-[4-[3-aminopropyl(methyl) carbamoyl]phenyl]-1,4-diazepan-1-yl]thieno[2,3-b]pyridine-2-carboxamide (20): To a 25 mL round-bottom flask was added a solution of tert-butyl N-[3-[[4-[4-(3-amino-2-carbamoyl-thieno[2,3-b]pyridin-4-yl)-1,4-diazepan-1-yl]benzoyl]-methyl-amino]propyl]carbamate (0.19 mmol, 113 mg) in DCM (5 mL). Then TFA (1 mL) was added and the mixture was stirred at room temperature for 4 h. Upon completion, the mixture was condensed and purified by flash column chromatography using a gradient of 0-9% MeOH/DCM to give 3-amino-4-[4-[4-[3-aminopropyl(methyl) carbamoyl]phenyl]-1,4-diazepan-1-yl]thieno[2,3-b]pyridine-2-carboxamide (77 mg, 82%). ESI-MS m/z: 482 ([M+H]⁺). ¹H-NMR (300 MHz, DMSO-d6): 8.41 (d, J=5.6 Hz, 1H), 8.26 (s, 1H), 7.31 (d, J=8.4 Hz, 2H), 7.11 (s, 2H), 7.08 (d, J=5.6 Hz, 1H), 6.97 (s, 2H), 6.79 (d, J=8.4 Hz, 2H), 3.82 (m, 2H), 3.60 (m, 2H), 3.46 (m, 2H), 3.31 (m, 2H), 3.21 (m, 2H), 2.98 (s, 3H), 2.79 (m, 2H), 2.15 (m, 2H), 1.85 (m, 2H).

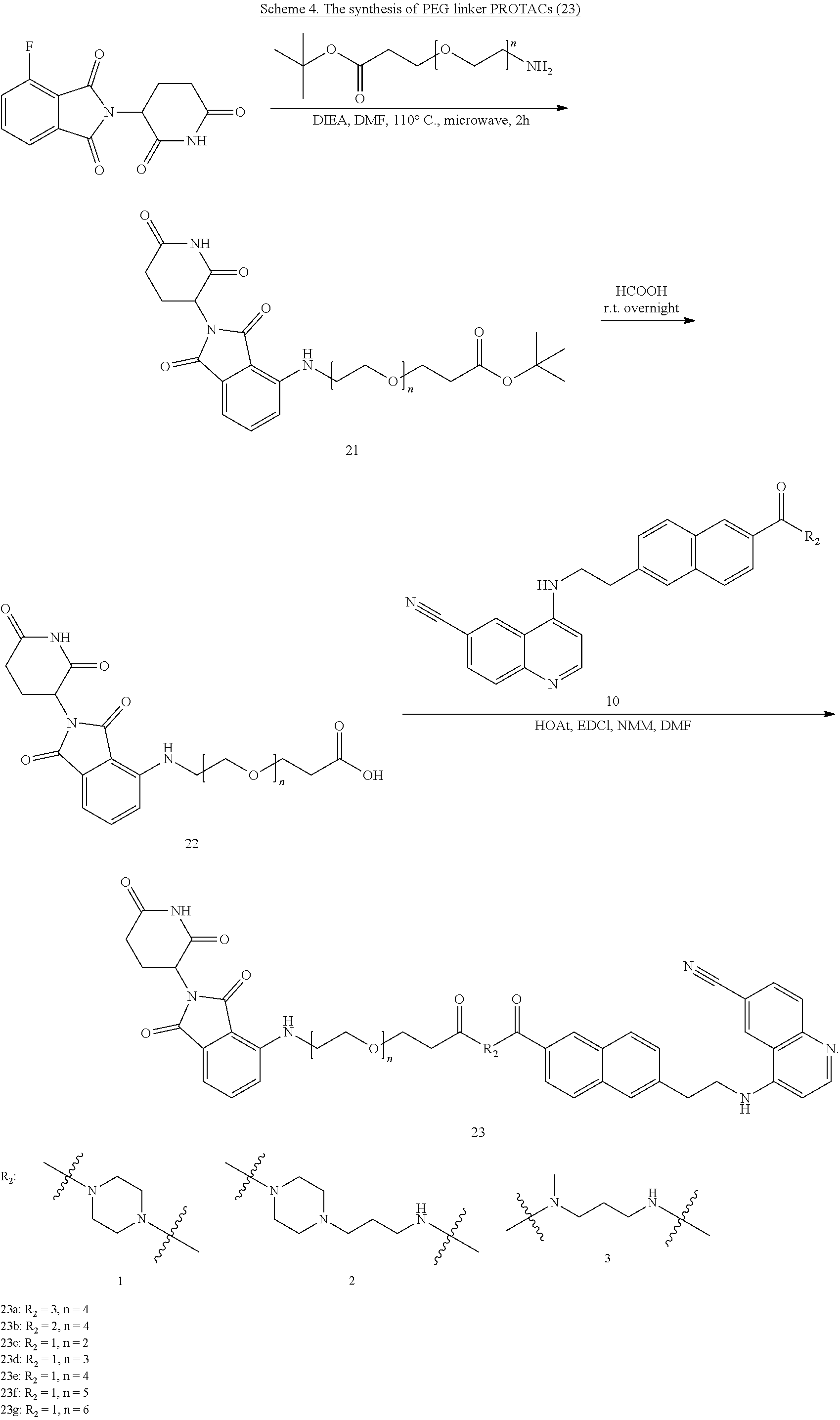

General method for 21: To a 10 mL microwave tube was added a solution of 2-(2,6-dioxo-3-piperidyl)-4-fluoro-isoindoline-1,3-dione (1 eq) in DMF. Then amine analogs (1 eq) and DIEA (3 eq) were added and the mixture was microwaved at 150° C. for 2 h. Upon completion, the mixture was condensed and purified by flash column chromatography using a gradient of 0-10% MeOH/DCM to give target compounds 21.

tert-butyl 3-[2-[2-[[2-(2,6-dioxo-3-piperidyl)-1,3-dioxo-isoindolin-4-yl]amino]ethoxy]ethoxy]propanoate (21a (n=2)): A yellow solid (216 mg. 49% yield). ESI-MS m/z: 490 ([M+H]⁺). ¹H-NMR (300 MHz, CD₂Cl₂): 8.35 (s, 1H), 7.51 (t, J=7.8 Hz, 1H), 7.07 (d, J=7.4 Hz, 1H), 6.96 (d, J=7.4 Hz, 1H), 6.48 (m, 1H), 4.91 (m, 1H), 3.69 (m, 4H), 3.61 (m, 4H), 3.46 (q, J=5.3 Hz, 2H), 2.81 (m, 2H), 2.73 (m, 1H), 2.45 (t, J=6.6 Hz, 2H), 2.12 (m, 1H), 1.42 (s, 9H).

tert-butyl 3-[2-[2-[2-[[2-(2,6-dioxo-3-piperidyl)-1,3-dioxo-isoindolin-4-yl]amino]ethoxy]ethoxy]ethoxy]propanoate (21b (n=3)): A yellow solid (199 mg, 47% yield). ESI-MS m/z: 534 ([M+H]⁺). ¹H-NMR (300 MHz, DMSO-d6): 11.07 (s, 1H), 7.58 (t, J=8.5 Hz, 1H), 7.15 (d, J=8.4 Hz, 1H), 7.04 (d, J=8.5 Hz, 1H), 6.60 (m, 1H), 5.06 (m, 1H), 3.61-3.47 (m, 14H), 2.88 (m, 1H), 2.57 (m, 2H), 2.39 (t, J=6.4 Hz, 2H), 2.02 (m, 1H), 1.38 (s, 9H).

tert-butyl 3-[2-[2-[2-[2-[[2-(2,6-dioxo-3-piperidyl)-1,3-dioxo-isoindolin-4-yl]amino]ethoxy]ethoxy]ethoxy]ethoxy]propanoate (21c (n=4)): A yellow solid (209 mg, 50% yield). ESI-MS m/z: 578 ([M+H]⁺). ¹H-NMR (300 MHz, DMSO-d6): 11.07 (s, 1H), 7.58 (t, J=8.5 Hz, 1H), 7.15 (d, J=8.4 Hz, 1H), 7.04 (d, J=8.5 Hz, 1H), 6.60 (m, 1H), 5.06 (m, 1H), 3.62-3.44 (m, 18H), 2.89 (m, 1H), 2.55 (m, 2H), 2.40 (t, J=6.4 Hz, 2H), 2.02 (m, 1H), 1.38 (s, 9H).

tert-butyl 3-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]propanoate (21d (n=5)): A yellow solid (254 mg, 51% yield). ESI-MS m/z: 622 ([M+H]⁺). ¹H-NMR (300 MHz, CD₂Cl₂): 8.59 (s, 1H), 7.51 (t, J=8.1 Hz, 1H), 7.07 (d, J=6.9 Hz, 1H), 6.96 (d, J=6.9 Hz, 1H), 6.52 (m, 1H), 4.91 (m, 1H), 3.69-3.45 (m, 22H), 2.77 (m, 3H), 2.45 (t, J=6.4 Hz, 2H), 2.10 (m, 1H), 1.43 (s, 9H).

tert-butyl 3-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethoxy]propanoate (21e (n=6)): A yellow solid (177 mg, 33% yield). ESI-MS m/z: 666 ([M+H]⁺). ¹H-NMR (300 MHz, CD₂Cl₂): 8.36 (s, 1H), 7.51 (t, J=8.1 Hz, 1H), 7.07 (d, J=6.9 Hz, 1H), 6.96 (d, J=6.9 Hz, 1H), 6.49 (m, 1H), 4.90 (m, 1H), 3.71-3.52 (m, 24H), 3.47 (m, 2H), 2.79 (m, 3H), 2.46 (t, J=6.4 Hz, 2H), 2.11 (m, 1H), 1.42 (s, 9H).

General method for 22: To a 10 mL round was added a solution of 21 (1 eq) in formic acid. Then the mixture was stirred at room temperature for overnight. Upon completion, the mixture was condensed and purified by flash column chromatography using a gradient of 0-10% MeOH/DCM to give target compounds 22.

3-[2-[2-[[2-(2,6-dioxo-3-piperidyl)-1,3-dioxo-isoindolin-4-yl]amino]ethoxy]ethoxy]propanoic acid (22a (n=2)): A yellow solid (186 mg, 97% yield). ESI-MS m/z: 434 ([M+H]⁺). ¹H-NMR (300 MHz, CD₂Cl₂): 8.76 (s, 1H), 7.51 (t, J=8.0 Hz, 1H), 7.07 (d, J=7.4 Hz, 1H), 6.95 (d, J=7.4 Hz, 1H), 6.50 (m, 1H), 4.93 (m, 1H), 3.71 (m, 4H), 3.64 (s, 4H), 3.46 (m, 2H), 2.78 (m, 3H), 2.60 (t, J=6.4 Hz, 2H), 2.11 (m, 1H).

3-[2-[2-[2-[[2-(2,6-dioxo-3-piperidyl)-1,3-dioxo-isoindolin-4-yl]amino]ethoxy]ethoxy]ethoxy]propanoic acid (22b (n=3)): A yellow solid (143 mg, 80% yield). ESI-MS m/z: 478 ([M+H]⁺). ¹H-NMR (300 MHz, CD₂Cl₂): 8.91 (s, 1H), 7.51 (t, J=8.0 Hz, 1H), 7.07 (d, J=7.4 Hz, 1H), 6.95 (d, J=7.4 Hz, 1H), 6.50 (m, 1H), 4.93 (m, 1H), 3.71 (m, 2H), 3.63 (s, 4H), 3.60 (s, 4H), 3.46 (m, 2H), 2.78 (m, 3H), 2.58 (t, J=6.4 Hz, 2H), 2.11 (m, 1H).

3-[2-[2-[2-[2-[[2-(2,6-dioxo-3-piperidyl)-1,3-dioxo-isoindolin-4-yl]amino]ethoxy]ethoxy]ethoxy]ethoxy]propanoic acid (22c (n=4)): A yellow solid (177 mg, 98% yield). ESI-MS m/z: 522 ([M+H]⁺). ¹H-NMR (300 MHz, DMSO-d6): 11.09 (s, 1H), 8.15 (s, 1H), 7.58 (t, J=8.3 Hz, 1H), 7.15 (d, J=8.9 Hz, 1H), 7.04 (d, J=7.2 Hz, 1H), 6.60 (m, 1H), 5.05 (m, 1H), 3.62-3.44 (m, 18H), 2.88 (m, 1H), 2.55 (m, 2H), 2.43 (t, J=6.4 Hz, 2H), 2.02 (m, 1H). 3-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]propanoic acid (22d (n=5)): A yellow solid (200 mg, 92% yield) and used without further purification. ESI-MS m/z: 566 ([M+H]⁺).

3-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethoxy]propanoic acid (22e (n=6)): A yellow solid (143 mg, 94% yield) and used without further purification. ESI-MS m/z: 610 ([M+H]⁺).

General method for 23: To a 10 mL round-bottle flask was added a solution of 22 (1eq) and 10 (1eq) in DMF. Then EDC-HCl (2 eq), HOAt (2 eq), and N-methylmorpholine (5 eq) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give target compounds 23.

6-[2-[(6-cyano-4-quinolyl)amino]ethyl]-N-[3-[3-[2-[2-[2-[2-[[2-(2,6-dioxo-3-piperidyl)-1,3-dioxo-isoindolin-4-yl]amino]ethoxy]ethoxy]ethoxy]ethoxy]propanoylamino]propyl]-N-methyl-naphthalene-2-carboxamide (23a (n=4, R₁=3)): A yellow solid (41 mg, 28% yield). ESI-MS m/z: 940 ([M+H]⁺). ¹H-NMR (300 MHz, CD₂Cl₂): 8.51 (m, 1H), 8.28 (m, 1H), 7.94 (d, J=8.1 Hz, 1H), 7.77 (m, 4H), 7.63 (s, 1H), 7.44 (t, J=8.1 Hz, 2H), 7.37 (d, J=8.1 Hz, 1H), 7.05 (m, 1H), 7.00 (d, J=7.5 Hz, 1H), 6.87 (d, J=8.1 Hz, 1H), 6.55 (m, 1H), 6.43 (m, 1H), 6.23 (m, 1H), 4.90 (m, 1H), 3.66-3.57 (m, 17H), 3.39 (m, 4H), 3.30 (m, 2H), 3.08 (m, 4H), 2.96 (m, 2H), 2.74 (m, 3H), 2.44 (m, 2H), 2.07 (m, 2H), 1.83 (m, 1H).

3-amino-4-[4-[4-[3-[3-[2-[2-[[2-(2,6-dioxo-3-piperidyl)-1,3-dioxo-isoindolin-4-yl]amino]ethoxy]ethoxy]propanoylamino]propyl-methyl-carbamoyl]phenyl]-1,4-diazepan-1-yl]thieno[2,3-b]pyridine-2-carboxamide (23b (n=4, R₁=2)): A yellow solid (58 mg, 56% yield). ESI-MS m/z: 898 ([M+H]⁺). ¹H-NMR (300 MHz, CD₃CN): 9.32 (s, 1H), 8.47 (d, J=5.5 Hz, 1H), 8.39 (s, 1H), 7.91-7.77 (m, 6H), 7.50-7.43 (m, 3H), 7.00 (d, J=6.9 Hz, 1H), 6.98 (d, J=2.2 Hz, 1H), 6.96 (d, J=4.3 Hz, 1H), 6.65 (d, J=6.0 Hz, 1H), 6.47 (m, 1H), 6.44 (t, J=5.6 Hz, 1H), 4.91 (m, 1H), 3.70 (m, 2H), 3.66 (t, J=5.6 Hz, 2H), 3.62 (t, J=5.6 Hz, 2H), 3.57 (m, 2H), 3.54 (m, 2H), 3.50 (s, 10H), 3.42 (q, J=5.6 Hz, 2H), 3.18 (m, 4H), 2.69 (m, 3H), 2.50 (m, 10H), 2.35 (t, J=5.6 Hz, 2H), 2.07 (m, 1H), 1.63 (m, 2H).

4-[2-[6-[4-[3-[2-[2-[[2-(2,6-dioxo-3-piperidyl)-1,3-dioxo-isoindolin-4-yl]amino]ethoxy]ethoxy]propanoyl]piperazine-1-carbonyl]-2-naphthyl]ethylamino]quinoline-6-carbonitrile (23c (n=2, R₁=1)): A yellow solid (42 mg, 21% yield). ESI-MS m/z: 852 ([M+H]⁺). ¹H-NMR (300 MHz, DMSO-d6): 11.1 (s, 1H), 8.87 (s, 1H), 8.52 (d, J=5.4 Hz, 1H), 7.91 (m, 6H), 7.76 (m, 1H), 7.56 (t, J=9.2 Hz, 2H), 7.49 (d, J=8.5 Hz, 1H), 7.11 (m, 1H), 7.02 (d, J=7.7 Hz, 1H), 6.73 (d, J=5.4 Hz, 1H), 6.58 (s, 1H), 5.05 (m, 1H), 3.70-3.45 (m, 20H), 3.17 (t, J=7.2 Hz, 2H), 2.87 (m, 1H), 2.56 (m, 4H), 2.01 (m, 1H).

4-[2-[6-[4-[3-[2-[2-[2-[[2-(2,6-dioxo-3-piperidyl)-1,3-dioxo-isoindolin-4-yl]amino]ethoxy]ethoxy]ethoxy]propanoyl]piperazine-1-carbonyl]-2-naphthyl]ethylamino]quinoline-6-carbonitrile (23d (n=3, R₁=1)): A yellow solid (36 mg, 27% yield). ESI-MS m/z: 896 ([M+H]⁺). ¹H-NMR (300 MHz, DMSO-d6): 11.1 (s, 1H), 8.87 (s, 1H), 8.52 (d, J=5.4 Hz, 1H), 7.93 (m, 6H), 7.76 (m, 1H), 7.56 (t, J=9.2 Hz, 2H), 7.49 (d, J=8.5 Hz, 1H), 7.11 (d, J=9.2 Hz, 1H), 7.02 (d, J=7.7 Hz, 1H), 6.72 (d, J=5.4 Hz, 1H), 6.58 (s, 1H), 5.05 (m, 1H), 3.75-3.56 (m, 24H), 3.18 (t, J=7.2 Hz, 2H), 2.87 (m, 1H), 2.56 (m, 4H), 2.00 (m, 1H).

4-[2-[6-[4-[3-[2-[2-[2-[2-[[2-(2,6-dioxo-3-piperidyl)-1,3-dioxo-isoindolin-4-yl]amino]ethoxy]ethoxy]ethoxy]ethoxy]propanoyl]piperazine-1-carbonyl]-2-naphthyl]ethylamino]quinoline-6-carbonitrile (23e (n=4, R₁=1)): A yellow solid (63 mg, 35% yield). ESI-MS m/z: 940 ([M+H]⁺). ¹H-NMR (300 MHz, CD₂Cl₂): 8.57 (d, J=5.4 Hz, 1H), 8.21 (d, J=6.9 Hz, 1H), 7.98 (d, J=8.9 Hz, 1H), 7.82 (m, 3H), 7.71 (d, J=8.9 Hz, 1H), 7.68 (s, 1H), 7.45 (m, 3H), 7.02 (d, J=6.9 Hz, 1H), 6.91 (d, J=8.7 Hz, 1H), 6.57 (d, J=5.4 Hz, 1H), 6.45 (t, J=5.4 Hz, 1H), 5.87 (m, 1H), 4.91 (m, 1H), 3.75-3.56 (m, 26H), 3.43 (m, 2H), 3.19 (m, 2H), 2.76 (m, 3H), 2.62 (m, 2H), 2.10 (m, 1H).

4-[2-[6-[4-[3-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]propanoyl]piperazine-1-carbonyl]-2-naphthyl]ethylamino]quinoline-6-carbonitrile (23f (n=5, R₁=1)): A yellow solid (15 mg, 17% yield). ESI-MS m/z: 954 ([M+H]⁺). ¹H-NMR (300 MHz, CD₂Cl₂): 8.94 (m, 1H), 8.60 (d, J=5.1 Hz, 1H), 8.21 (s, 1H), 8.02 (d, J=8.9 Hz, 1H), 7.88 (s, 1H), 7.85 (d, J=8.3 Hz, 1H), 7.81 (d, J=8.3 Hz, 1H), 7.72 (d, J=8.6 Hz, 1H), 7.69 (s, 1H), 7.48 (d, J=8.0 Hz, 2H), 7.43 (d, J=8.9 Hz, 1H), 7.03 (d, J=7.2 Hz, 1H), 6.91 (d, J=8.0 Hz, 1H), 6.58 (d, J=5.6 Hz, 1H), 6.46 (t, J=6.4 Hz, 1H), 5.91 (m, 1H), 4.90 (m, 1H), 3.75-3.56 (m, 30H), 3.44 (m, 2H), 3.20 (t, J=7.6 Hz, 2H), 2.77 (m, 3H), 2.63 (m, 2H), 2.10 (m, 1H).

4-[2-[6-[4-[3-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethoxy]propanoyl]piperazine-1-carbonyl]-2-naphthyl]ethylamino]quinoline-6-carbonitrile (23 g (n=6, R₁=1)): A yellow solid (30 mg, 36% yield). ESI-MS m/z: 1028 ([M+H]⁺). ¹H-NMR (300 MHz, CD₂Cl₂): 8.94 (m, 1H), 8.60 (d, J=5.1 Hz, 1H), 8.17 (s, 1H), 8.00 (d, J=8.9 Hz, 1H), 7.88 (s, 1H), 7.86 (d, J=8.3 Hz, 1H), 7.83 (d, J=8.3 Hz, 1H), 7.72 (d, J=8.6 Hz, 1H), 7.71 (s, 1H), 7.49 (d, J=8.0 Hz, 2H), 7.44 (d, J=8.9 Hz, 1H), 7.04 (d, J=7.2 Hz, 1H), 6.92 (d, J=8.0 Hz, 1H), 6.59 (d, J=5.6 Hz, 1H), 6.47 (t, J=6.4 Hz, 1H), 5.91 (m, 1H), 4.90 (m, 1H), 3.75-3.56 (m, 34H), 3.44 (m, 2H), 3.21 (t, J=7.6 Hz, 2H), 2.77 (m, 3H), 2.63 (m, 2H), 2.11 (m, 1H)

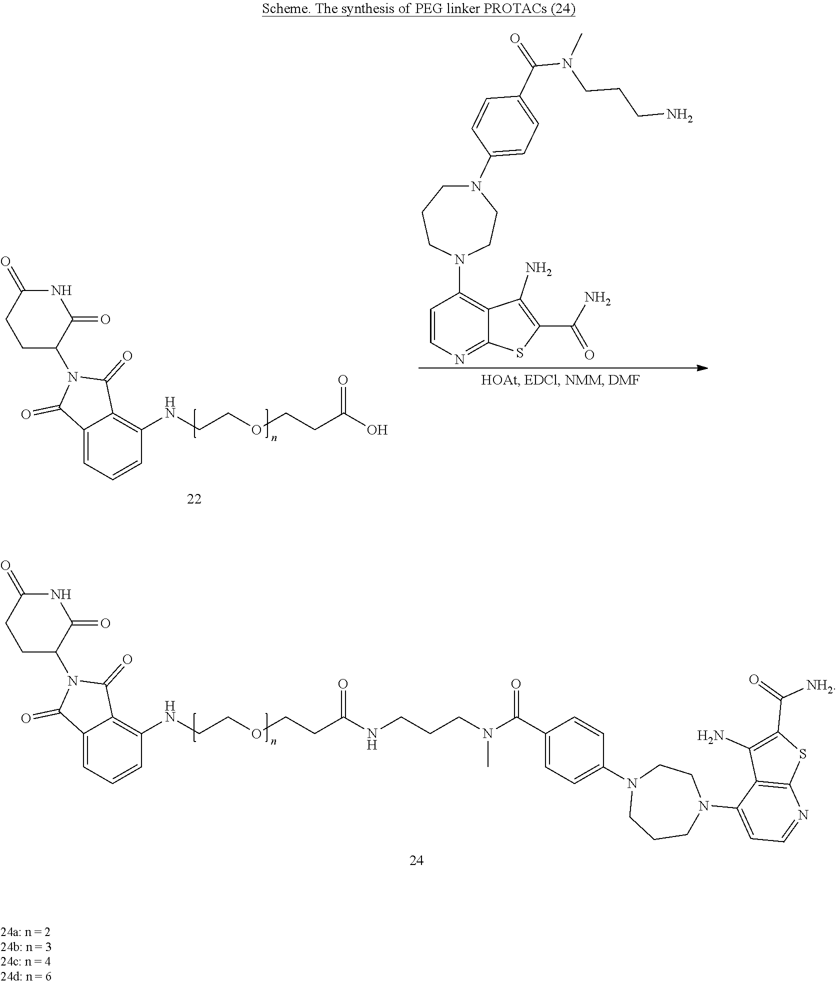

General method for 24: To a 10 mL round-bottle flask was added a solution of 22 (1eq) and 20 (1eq) in DMF. Then EDC-HCl (2 eq), HOAt (2 eq), and N-methylmorpholine (Seq) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give target compounds 24.

3-amino-4-[4-[4-[3-[3-[2-[2-[[2-(2,6-dioxo-3-piperidyl)-1,3-dioxo-isoindolin-4-yl]amino]ethoxy]ethoxy]propanoylamino]propyl-methyl-carbamoyl]phenyl]-1,4-diazepan-1-yl]thieno[2,3-b]pyridine-2-carboxamide (24a (n=2)): A yellow solid (58 mg, 56% yield). ESI-MS m/z: 898 ([M+H]⁺). ¹H-NMR (300 MHz, DMSO-d6): 9.50 (s, 1H), 8.44 (d, J=5.4 Hz, 1H), 8.07 (s, 1H), 7.49 (t, J=8.1 Hz, 1H), 7.35 (d, J=8.7 Hz, 2H), 7.06 (d, J=7.5 Hz, 1H), 6.96 (d, J==5.4 Hz, 1H), 6.93 (d, J=8.7 Hz, 2H), 6.91 (d, J=7.5 Hz, 1H), 6.73 (d, J=8.7 Hz, 2H), 6.51 (m, 1H), 5.58 (m, 2H), 4.91 (m, 1H), 3.79 (m, 2H), 3.70 (m, 4H), 3.62 (m, 6H), 3.51 (m, 2H), 3.44 (m, 1H), 3.36 (m, 2H), 3.27 (m, 2H), 3.19 (m, 2H), 3.00 (s, 3H), 2.76 (m, 3H), 2.40 (m, 2H), 2.17 (m, 2H), 2.10 (m, 1H), 1.76 (m, 2H).

3-amino-4-[4-[4-[3-[3-[2-[2-[2-[[2-(2,6-dioxo-3-piperidyl)-1,3-dioxo-isoindolin-4-yl]amino]ethoxy]ethoxy]ethoxy]propanoylamino]propyl-methyl-carbamoyl]phenyl]-1,4-diazepan-1-yl]thieno[2,3-b]pyridine-2-carboxamide (24b (n=3)): A yellow solid (60 mg, 61% yield). ESI-MS m/z: 942 ([M+H]⁺). ¹H-NMR (300 MHz, DMSO-d6): 9.40 (s, 1H), 8.44 (d, J=5.4 Hz, 1H), 8.07 (s, 1H), 7.47 (t, J=8.1 Hz, 1H), 7.34 (d, J=8.7 Hz, 2H), 7.04 (d, J=7.5 Hz, 1H), 6.96 (d, J=5.4 Hz, 1H), 6.93 (m, 3H), 6.73 (d, J=8.7 Hz, 2H), 6.49 (m, 1H), 5.66 (m, 2H), 4.92 (m, 1H), 3.78 (m, 2H), 3.69 (m, 4H), 3.62 (m, 6H), 3.59 (m, 4H), 3.52 (m, 2H), 3.44 (m, 2H), 3.36 (m, 2H), 3.27 (m, 2H), 3.19 (m, 2H), 3.01 (s, 3H), 2.75 (m, 3H), 2.40 (m, 2H), 2.17 (m, 2H), 2.10 (m, 1H), 1.78 (m, 2H).

3-amino-4-[4-[4-[3-[3-[2-[2-[2-[2-[[2-(2,6-dioxo-3-piperidyl)-1,3-dioxo-isoindolin-4-yl]amino]ethoxy]ethoxy]ethoxy]ethoxy]propanoylamino]propyl-methyl-carbamoyl]phenyl]-1,4-diazepan-1 (24c (n=4)): A yellow solid (95 mg, 56% yield). ESI-MS m/z: 986 ([M+H]⁺). ¹H-NMR (300 MHz, DMSO-d6): 11.09 (s, 1H), 8.40 (d, J=5.4 Hz, 1H), 7.81 (s, 1H), 7.57 (t, J=7.9 Hz, 1H), 7.26 (d, J=8.6 Hz, 2H), 7.15-7.00 (m, 6H), 6.77 (d, J=8.6 Hz, 2H), 6.60 (m, 1H), 5.05 (m, 1H), 3.81 (m, 2H), 3.60-3.42 (m, 24H), 3.20 (m, 2H), 3.02 (m, 2H), 2.93 (s, 3H), 2.86 (m, 1H), 2.57 (m, 2H), 2.26 (m, 2H), 2.16 (m, 2H), 2.01 (m, 1H), 1.68 (m, 2H).

3-amino-4-[4-[4-[3-[3-[2-[2-[2-[2-[2-[2-[[2-(2,6-dioxo-3-piperidyl)-1,3-dioxo-isoindolin-4-yl]amino]ethoxy]ethoxy]ethoxy]ethoxy]ethoxy]ethoxy]propanoylamino]propyl-methyl-carbamoyl]phenyl]-1,4-diazepan-1-yl]thieno[2,3-b]pyridine-2-carboxamide (24d (n=6)): A yellow solid (51 mg, 58% yield). ESI-MS m/z: 1074 ([M+H]⁺). ¹H-NMR (300 MHz, CD₂Cl₂): 9.04 (s, 1H), 8.45 (d, J=5.9 Hz, 1H), 7.50 (t, J=7.7 Hz, 1H), 7.34 (d, J=8.9 Hz, 2H), 7.06 (d, J=7.1 Hz, 1H), 6.95 (m, 4H), 6.74 (d, J=8.9 Hz, 2H), 6.49 (t, J=5.9 Hz, 1H), 5.59 (m, 2H), 4.91 (m, 1H), 3.80 (d, J=5.5 Hz, 2H), 3.71-3.58 (m, 28H), 3.50-3.36 (m, 6H), 3.28 (m, 2H), 3.20 (m, 2H), 3.02 (s, 3H), 2.77 (m, 3H), 2.40 (m, 2H), 2.17 (m, 2H), 2.10 (m, 1H).

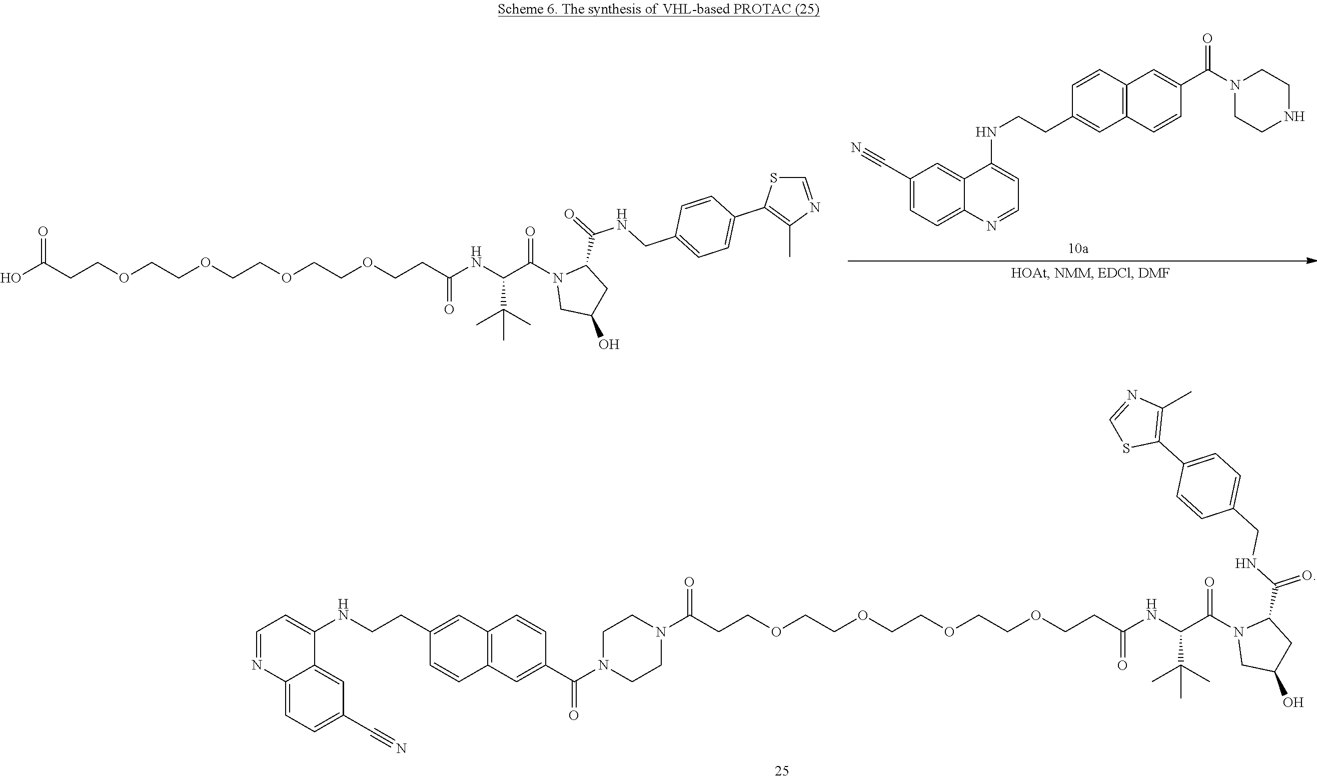 [SMILES: Cc1ncsc1-c1ccc(CNC(=O)[C@@H]2C[C@@H](O)CN2C(=O)[C@@H](NC(=O)CCOCCOCCOCCOCCC(=O)O)C(C)(C)C)cc1 ; N#Cc1ccc2nccc(NCCc3ccc4cc(C(=O)N5CCNCC5)ccc4c3)c2c1 ; Cc1ncsc1-c1ccc(CNC(=O)[C@@H]2C[C@@H](O)CN2C(=O)[C@@H](NC(=O)CCOCCOCCOCCOCCC(=O)N2CCN(C(=O)c3ccc4cc(CCNc5ccnc6ccc(C#N)cc56)ccc4c3)CC2)C(C)(C)C)cc1]

(2S,4R)-1-((S)-2-(tert-butyl)-19-(4-(6-(2-((6-cyanoquinolin-4-yl)amino)ethyl)-2-naphthoyl) piperazin-1-yl)-4,19-dioxo-7,10,13,16-tetraoxa-3-azanonadecanoyl)-4-hydroxy-N-(4-(4-methylthiazol-5-yl) benzyl) pyrrolidine-2-carboxamide (25): To a 10 mL round-bottle flask was added a solution of 3-[2-[2-[2-[3-oxo-3-[[rac-(1S)-2,2-dimethyl-1-[rac-(2S,4R)-4-hydroxy-2-[[4-(4-methylthiazol-5-yl)phenyl]methylcarbamoyl]pyrrolidine-1-carbonyl]propyl]amino]propoxy]ethoxy]ethoxy]ethoxy]propanoic acid (0.071 mmol, 50 mg) and 4-[2-[6-(piperazine-1-carbonyl)-2-naphthyl]ethylamino]quinoline-6-carbonitrile (0.071 mmol, 31 mg) in DMF (2 mL). Then EDC-HCl (0.14 mmol, 27 mg), HOAt (0.14 mmol, 19 mg), and N-methylmorpholine (0.35 mmol, 36 mg) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give (2S,4R)-1-((S)-2-(tert-butyl)-19-(4-(6-(2-((6-cyanoquinolin-4-yl)amino)ethyl)-2-naphthoyl) piperazin-1-yl)-4,19-dioxo-7,10,13,16-tetraoxa-3-azanonadecanoyl)-4-hydroxy-N-(4-(4-methylthiazol-5-yl) benzyl) pyrrolidine-2-carboxamide (21 mg, 26% yield). ESI-MS m/z: 1125 ([M+H]⁺). ¹H-NMR (300 MHz, CD₂Cl₂): 8.63 (s, 1H), 8.56 (d, J=5.1 Hz, 1H), 8.31 (s, 1H), 7.97 (d, J=8.6 Hz, 1H), 7.86 (s, 1H), 7.83 (d, J=8.6 Hz, 1H), 7.80 (d, J=8.6 Hz, 1H), 7.72 (dd, J=8.6, 1.5 Hz, 1H), 7.68 (s, 1H), 7.44 (m, 3H), 7.34 (m, 4H), 7.03 (d, J=8.6 Hz, 1H), 6.59 (d, J=5.5 Hz, 1H), 6.26 (m, 1H), 4.63 (t, J=8.1 Hz, 2H), 4.49 (m 3H), 4.28 (m, 1H), 3.99 (m, 1H), 3.75-3.55 (m, 28H), 3.20 (t, J=: 7.2 Hz, 2H), 2.62 (m, 2H), 2.45 (m, 2H), 2.44 (s, 3H), 2.34 (m, 1H), 2.09 (m, 1H), 0.93 (s, 9H).

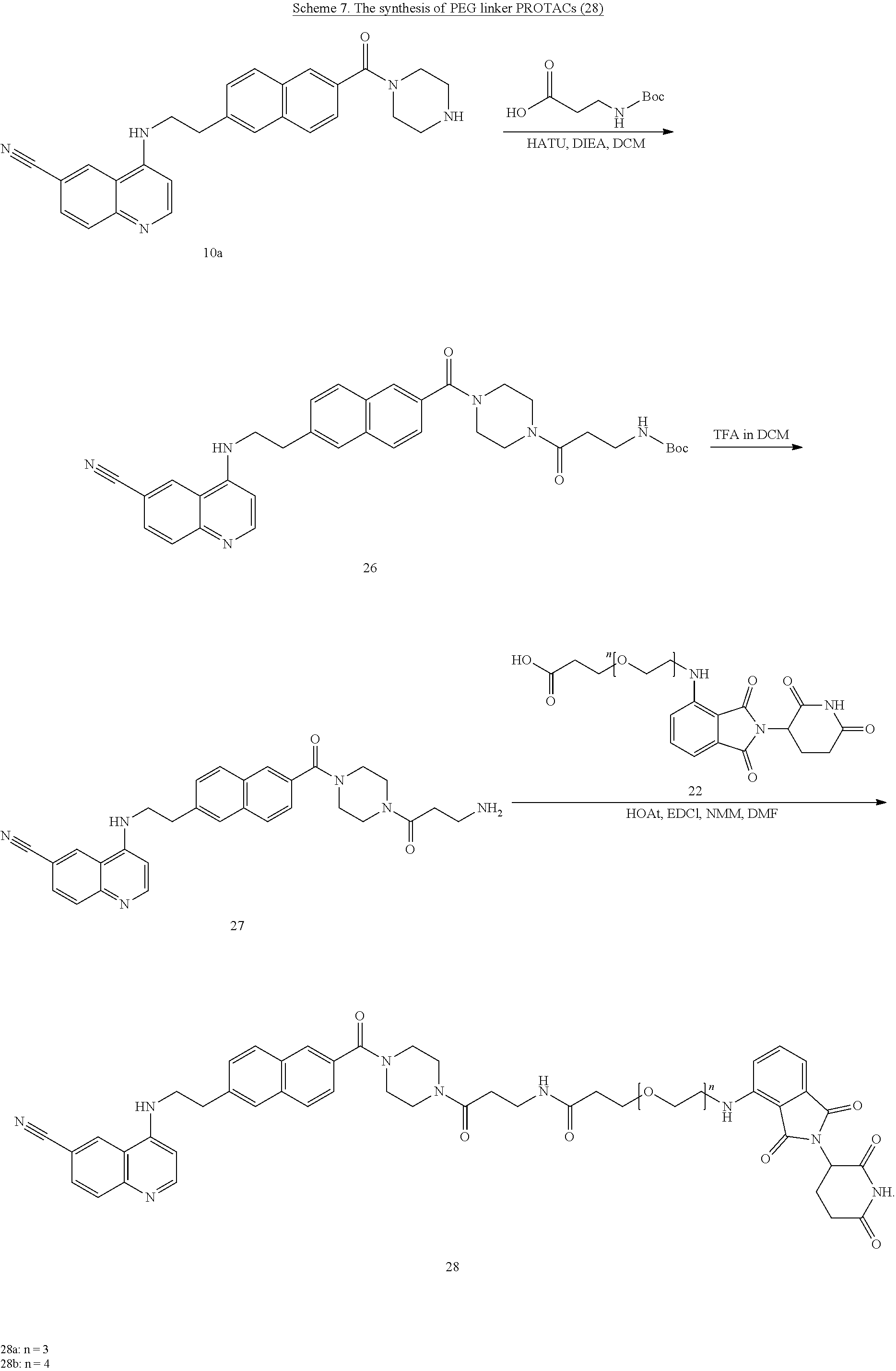

tert-butyl N-[3-[4-[6-[2-[(6-cyano-4-quinolyl)amino]ethyl]naphthalene-2-carbonyl]piperazin-1-yl]-3-oxo-propyl]carbamate (26): To a 10 mL round-bottle flask was added a solution of 3-(tert-butoxycarbonylamino) propanoic acid (0.52 mmol, 99 mg) in DCM (5 mL). Then HATU (0.79 mmol, 298 mg), DIEA (1.57 mmol, 269 μL) were added and the mixture was room temperature for 15 min, then 4-[2-[6-(piperazine-1-carbonyl)-2-stirred at naphthyl]ethylamino]quinoline-6-carbonitrile (0.52 mmol, 228 mg) was added and the mixture was stirred for 2 h. Upon completion, water was added, the mixture was extracted with DCM, the organic layers were combined, washed with sat NaHCO₃ aq, dried by Na₂SO₄, condensed and purified by flash column chromatography using a gradient of 0-5% MeOH/DCM to give tert-butyl N-[3-[4-[6-[2-[(6-cyano-4-quinolyl)amino]ethyl]naphthalene-2-carbonyl]piperazin-1-yl]-3-oxo-propyl]carbamate (89 mg, 28% yield). ESI-MS m/z: 607 ([M+H]⁺). ¹H-NMR (300 MHz, CD₂Cl₂): 8.58 (d, J=5.6 Hz, 1H), 8.23 (s, 1H), 8.02 (d, J=8.6 Hz, 1H), 7.88 (s, 1H), 7.84 (t, J=8.1 Hz, 2H), 7.75 (dd, J=8.8, 1.7 Hz, 1H), 7.68 (s, 1H), 7.48 (dd, J=8.8, 1.7 Hz, 1H), 7.43 (dd, J=8.8, 1.7 Hz, 1H), 6.61 (d, J=5.7 Hz, 1H), 6.06 (m, 1H), 5.25 (m, 1H), 4.01 (m, 2H), 3.70 (m, 2H), 3.62 (m, 2H), 3.51 (m, 2H), 3.37 (m, 4H), 3.21 (t, J=6.3 Hz, 2H), 2.52 (m, 4H), 1.41 (s, 9H).

4-[2-[6-[4-(3-aminopropanoyl) piperazine-1-carbonyl]-2-naphthyl]ethylamino]quinoline-6-carbonitrile (27): To a 10 mL round-bottle flask was added a solution of tert-butyl N-[3-[4-[6-[2-[(6-cyano-4-quinolyl)amino]ethyl]naphthalene-2-carbonyl]piperazin-1-yl]-3-oxo-propyl]carbamate (0.14 mmol, 86 mg) in DCM (5 mL). Then TFA (1 mL) was added and the mixture was stirred at room temperature for 4 h. Upon completion, the mixture was condensed to give 4-[2-[6-[4-(3-aminopropanoyl) piperazine-1-carbonyl]-2-naphthyl]ethylamino]quinoline-6-carbonitrile (65 mg, 91% yield) and used without further purification. ESI-MS m/z: 507 ([M+H]⁺).

N-[3-[4-[6-[2-[(6-cyano-4-quinolyl)amino]ethyl]naphthalene-2-carbonyl]piperazin-1-yl]-3-oxo-propyl]-3-[2-[2-[2-[[2-(2,6-dioxo-3-piperidyl)-1,3-dioxo-isoindolin-4-yl]amino]ethoxy]ethoxy]ethoxy]propanamide (28a (n=3)): To a 10 mL round-bottle flask was added a solution of 3-[2-[2-[2-[[2-(2,6-dioxo-3-piperidyl)-1,3-dioxo-isoindolin-4-yl]amino]ethoxy]ethoxy]ethoxy]propanoic acid (0.10 mmol, 50 mg) and 4-[2-[6-[4-(3-aminopropanoyl) piperazine-1-carbonyl]-2-naphthyl]ethylamino]quinoline-6-carbonitrile (0.10 mmol, 53 mg) in DMF (2 mL). Then EDC-HCl (0.21 mmol, 40 mg), HOAt (0.21 mmol, 29 mg), and N-methylmorpholine (0.52 mmol, 53 mg) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give N-[3-[4-[6-[2-[(6-cyano-4-quinolyl)amino]ethyl]naphthalene-2-carbonyl]piperazin-1-yl]-3-oxo-propyl]-3-[2-[2-[2-[[2-(2,6-dioxo-3-piperidyl)-1,3-dioxo-isoindolin-4-yl]amino]ethoxy]ethoxy]ethoxy]propanamide (15 mg, 15% yield). ESI-MS m/z: 967 ([M+H]⁺). H-NMR (300 MHz, CD₂Cl₂): 9.59 (m, 1H), 8.59 (d, J=5.1 Hz, 1H), 8.22 (s, 1H), 7.97 (d, J=9.2 Hz, 1H), 7.86 (s, 1H), 7.82 (t, J=8.8 Hz, 2H), 7.70 (d, J=10.6 Hz, 2H), 7.72 (d, J=8.6 Hz, 1H), 7.71 (s, 1H), 7.46 (m, 3H), 7.03 (d, J=7.4 Hz, 1H), 6.91 (d, J=8.3 Hz, 2H), 6.57 (d, J=5.5 Hz, 1H), 6.48 (d, J=5.4 Hz, 1H), 5.89 (m, 1H), 4.91 (m, 1H), 3.71-3.42 (m, 24H), 3.19 (t, J=7.5 Hz, 2H), 2.75 (m, 3H), 2.53 (m, 2H), 2.36 (t, J=6.6 Hz, 2H), 2.09 (m, 2H), 2.01 (m, 1H).

N-[3-[4-[6-[2-[(6-cyano-4-quinolyl)amino]ethyl]naphthalene-2-carbonyl]piperazin-1-yl]-3-oxo-propyl]-3-[2-[2-[2-[2-[[2-(2,6-dioxo-3-piperidyl)-1,3-dioxo-isoindolin-4-yl]amino]ethoxy]ethoxy]ethoxy]ethoxy]propanamide (28b (n=4): The method is the same as 28a. A yellow solid (15 mg, 16% yield). ESI-MS m/z: 1011 ([M+H]⁺). ¹H-NMR (300 MHz, CD₂Cl): 9.20 (m, 1H), 8.61 (d, J=5.1 Hz, 1H), 8.15 (s, 1H), 7.99 (d, J=9.2 Hz, 1H), 7.88 (s, 1H), 7.83 (t, J=8.8 Hz, 2H), 7.73 (d, J=10.6 Hz, 2H), 7.72 (d, J=8.6 Hz, 1H), 7.71 (s, 1H), 7.47 (m, 3H), 7.04 (d, J=7.4 Hz, 1H), 6.92 (d, J=8.3 Hz, 1H), 6.88 (m, 1H), 6.60 (d, J=5.5 Hz, 1H), 6.48 (t, J=5.4 Hz, 1H), 5.61 (m, 1H), 4.91 (m, 1H), 3.71-3.43 (m, 28H), 3.22 (t, J=7.5 Hz, 2H), 2.76 (m, 3H), 2.53 (m, 2H), 2.37 (t, J=6.6 Hz, 2H), 2.12 (m, 2H), 2.05 (m, 1H).

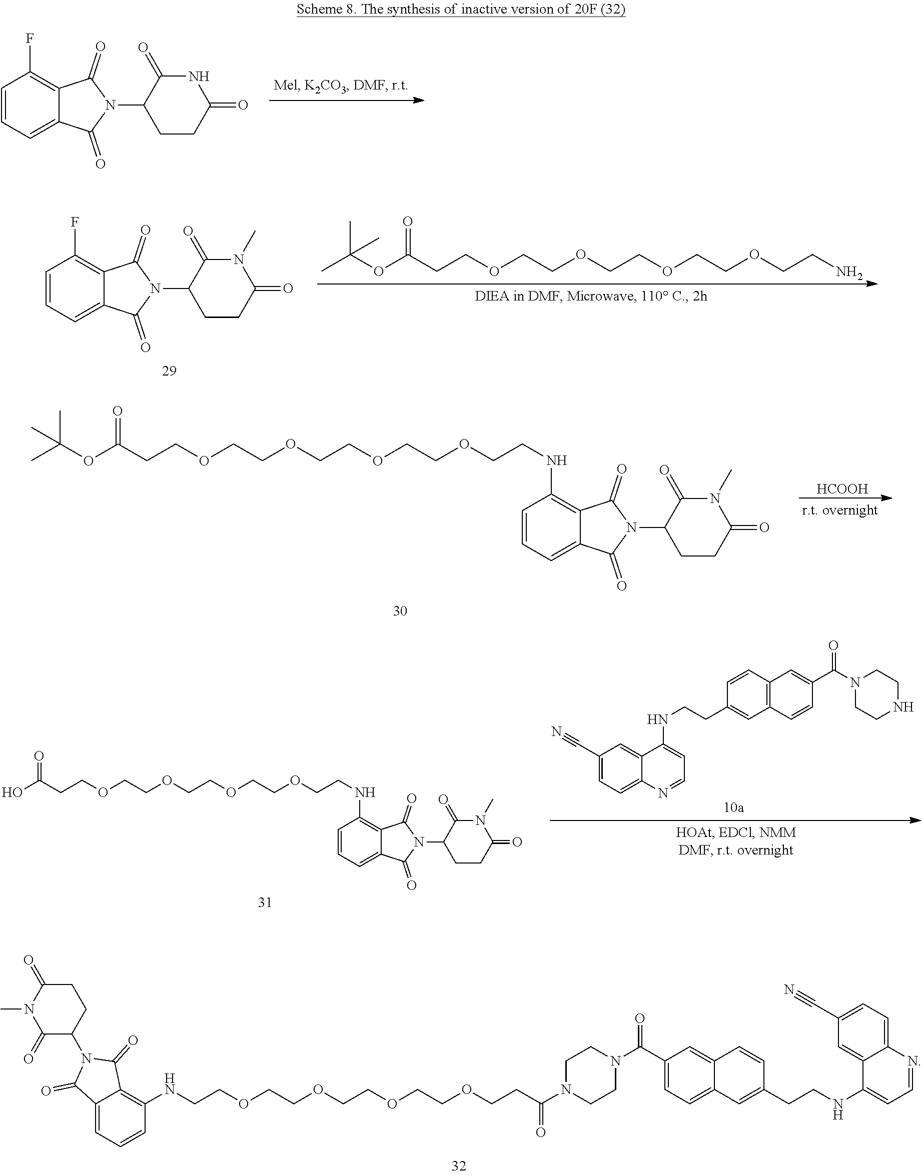

4-fluoro-2-(1-methyl-2,6-dioxo-3-piperidyl) isoindoline-1,3-dione (29): To a 10 mL round-bottle flask was added a solution of 2-(2,6-dioxo-3-piperidyl)-4-fluoro-isoindoline-1,3-dione (1.81 mmol, 500 mg), iodomethane (1.99 mmol, 283 mg), and K₂CO₃ (1.99 mmol, 275 mg) in DMF (5 mL). The mixture was stirred at room temperature for 7 h. Upon completion, the mixture was quenched by water, extracted with DCM, the organic layers were combined, dried by Na₂SO₄, condensed and purified by flash column chromatography using a gradient of 0-5% MeOH/DCM to give 4-fluoro-2-(1-methyl-2,6-dioxo-3-piperidyl) isoindoline-1,3-dione (500 mg, 95% yield). ESI-MS m/z: 291 ([M+H]⁺). ¹H-NMR (300 MHz, CD₂Cl₂): 7.81 (m, 1H), 7.73 (d, J=7.3 Hz, 1H), 7.47 (t, J=8.7 Hz, 1H), 5.01 (m, 1H), 3.19 (s, 3H), 2.96 (m, 1H), 2.80 (m, 2H), 2.15 (m, 1H).

tert-butyl 3-[2-[2-[2-[2-[[2-(1-methyl-2,6-dioxo-3-piperidyl)-1,3-dioxo-isoindolin-4-yl]amino]ethoxy]ethoxy]ethoxy]ethoxy]propanoate (30): To a 10 mL microwave tube was added a solution of 4-fluoro-2-(1-methyl-2,6-dioxo-3-piperidyl) isoindoline-1,3-dione (1.03 mmol, 300 mg), tert-butyl 3-[2-[2-[2-(2-aminoethoxy) ethoxy]ethoxy]ethoxy]propanoate (1.03 mmol, 332 mg), and DIEA (3.1 mmol, 401 mg) in DMF (5 mL). The mixture was microwaved at 110° C. for 2 h. Upon completion, the mixture was condensed and purified by flash column chromatography using a gradient of 0-9% MeOH/DCM to give tert-butyl 3-[2-[2-[2-[2-[[2-(1-methyl-2,6-dioxo-3-piperidyl)-1,3-dioxo-isoindolin-4-yl]amino]ethoxy]ethoxy]ethoxy]ethoxy]propanoate (500 mg, 82% yield). ESI-MS m/z: 291 ([M+H]⁺). ¹H-NMR (300 MHz, CD₂Cl₂): 7.52 (t, J=7.8 Hz, 1H), 7.07 (d, J=7.0 Hz, 1H), 6.97 (d, J=7.0 Hz, 1H), 6.48 (m, 1H), 4.91 (m, 1H), 3.60 (m, 18H), 3.15 (s, 3H), 2.92 (m, 1H), 2.75 (m, 2H), 2.57 (t, J=6.3 Hz, 2H), 2.08 (m, 2H).

3-[2-[2-[2-[2-[[2-(1-methyl-2,6-dioxo-3-piperidyl)-1,3-dioxo-isoindolin-4-yl]amino]ethoxy]ethoxy]ethoxy]ethoxy]propanoic acid (31): To a 10 mL round-bottom flask was added a solution of tert-butyl 3-[2-[2-[2-[2-[[2-(1-methyl-2,6-dioxo-3-piperidyl)-1,3-dioxo-isoindolin-4-yl]amino]ethoxy]ethoxy]ethoxy]ethoxy]propanoate (0.85 mmol, 500 mg) in formic acid (3 mL). The mixture was stirred at room temperature for overnight. Upon completion, the mixture was condensed to give 3-[2-[2-[2-[2-[[2-(1-methyl-2,6-dioxo-3-piperidyl)-1,3-dioxo-isoindolin-4-yl]amino]ethoxy]ethoxy]ethoxy]ethoxy]propanoic acid (332 mg, 73% yield) and used without further purification. ESI-MS m/z: 536 ([M+H]⁺). 4-[2-[6-[4-[3-[2-[2-[2-[2-[[2-(1-methyl-2,6-dioxo-3-piperidyl)-1,3-dioxo-isoindolin-4-yl]amino]ethoxy]ethoxy]ethoxy]ethoxy]propanoyl]piperazine-1-carbonyl]-2-naphthyl]ethylamino]quinoline-6-carbonitrile (32): To a 10 mL round-bottle flask was added a solution of 3-[2-[2-[2-[2-[[2-(1-methyl-2,6-dioxo-3-piperidyl)-1,3-dioxo-isoindolin-4-yl]amino]ethoxy]ethoxy]ethoxy]ethoxy]propanoic acid (0.32 mmol, 170 mg) and 4-[2-[6-(piperazine-1-carbonyl)-2-naphthyl]ethylamino]quinoline-6-carbonitrile (0.32 mmol, 138 mg) in DMF (3 mL). Then EDC-HCl (0.64 mmol, 122 mg), HOAt (0.64 mmol, 86 mg), and N-methylmorpholine (1.6 mmol, 161 mg) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give 4-[2-[6-[4-[3-[2-[2-[2-[2-[[2-(1-methyl-2,6-dioxo-3-piperidyl)-1,3-dioxo-isoindolin-4-yl]amino]ethoxy]ethoxy]ethoxy]ethoxy]propanoyl]piperazine-1-carbonyl]-2-naphthyl]ethylamino]quinoline-6-carbonitrile (54 mg, 18% yield). ESI-MS m/z: 954 ([M+H]⁺). 1H-NMR (300 MHz, CD₂Cl₂): 8.55 (d, J=5.1 Hz, 1H), 8.23 (s, 1H), 7.95 (d, J=8.5 Hz, 1H), 7.84 (m, 3H), 7.74 (d, J=9.1 Hz, 1H), 7.69 (s, 1H), 7.45 (m, 3H), 7.02 (d, J=7.4 Hz, 1H), 6.91 (d, J=8.5 Hz, 1H), 6.61 (d, J=6.7 Hz, 1H), 6.43 (m, 1H), 5.95 (m, 1H), 4.90 (m, 1H), 3.75-3.53 (m, 26H), 3.45 (m, 2H), 3.20 (m, 2H), 3.13 (s, 3H), 2.90 (m, 1H), 2.72 (m, 2H), 2.62 (m, 2H), 2.07 (m, 1H).

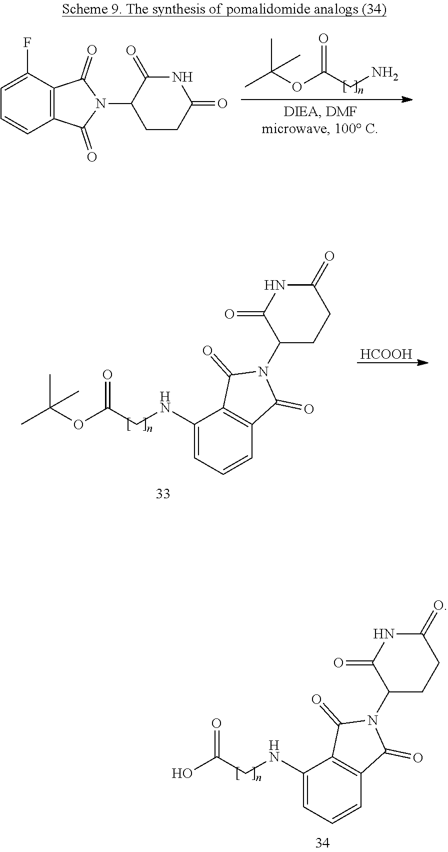

General method for 33: To a 10 mL round-bottle flask was added a solution of 2-(2,6-dioxo-3-piperidyl)-4-fluoro-isoindoline-1,3-dione (1eq) and amine analogs (1eq) in DMF. Then DIEA (3 eq) was added and the mixture was microwaved at 100° C. for 2 h. Upon completion, the mixture was condensed and purified by flash column chromatography using a gradient of 0-2% MeOH/DCM to give target compounds 33.

tert-butyl 3-[[2-(2,6-dioxo-3-piperidyl)-1,3-dioxo-isoindolin-4-yl]amino]propanoate (33a (n=2)): A yellow solid (70 mg, 24% yield). ESI-MS m/z: 402 ([M+H]⁺).

tert-butyl 4-[[2-(2,6-dioxo-3-piperidyl)-1,3-dioxo-isoindolin-4-yl]amino]butanoate (33b (n=3)): To a 10 mL round-bottle flask was added a solution of 2-(2,6-dioxo-3-piperidyl)-4-fluoro-isoindoline-1,3-dione (0.72 mmol, 200 mg) and tert-butyl 4-aminobutanoate (0.72 mmol, 115 mg) in DMF (2 mL). Then DIEA (2.2 mmol, 372 μL) was added and the mixture was microwaved at 100° C. for 2 h. Upon completion, the mixture was condensed and purified by flash column chromatography using a gradient of 0-2% MeOH/DCM to give tert-butyl 4-[[2-(2,6-dioxo-3-piperidyl)-1,3-dioxo-isoindolin-4-yl]amino]butanoate (70 mg, 23% yield). ESI-MS m/z: 416 ([M+H]⁺).

tert-butyl 5-[[2-(2,6-dioxo-3-piperidyl)-1,3-dioxo-isoindolin-4-yl]amino]pentanoate (33c (n=4)): To a 10 mL microwave flask was added a solution of 2-(2,6-dioxo-3-piperidyl)-4-fluoro-isoindoline-1,3-dione (0.72 mmol, 200 mg) and tert-butyl 5-aminopentanoate (0.72 mmol, 125 mg) in DMF (2 mL). Then DIEA (2.2 mmol, 372 μL) was added and the mixture was microwaved at 100° C. for 2 h. Upon completion, the mixture was condensed and purified by flash column chromatography using a gradient of 0-5% MeOH/DCM to give tert-butyl 5-[[2-(2,6-dioxo-3-piperidyl)-1,3-dioxo-isoindolin-4-yl]amino]pentanoate (143 mg, 46% yield). ESI-MS m/z: 430 ([M+H]⁺).

tert-butyl 6-[[2-(2,6-dioxo-3-piperidyl)-1,3-dioxo-isoindolin-4-yl]amino]hexanoate (33d (n=5)): To a 10 mL microwave flask was added a solution of 2-(2,6-dioxo-3-piperidyl)-4-fluoro-isoindoline-1,3-dione (0.72 mmol, 200 mg) and tert-butyl 6-aminohexanoate (0.72 mmol, 136 mg) in DMF (2 mL). Then DIEA (2.2 mmol, 372 μL) was added and the mixture was microwaved at 100° C. for 2 h. Upon completion, the mixture was condensed and purified by flash column chromatography using a gradient of 0-5% MeOH/DCM to give tert-butyl 6-[[2-(2,6-dioxo-3-piperidyl)-1,3-dioxo-isoindolin-4-yl]amino]hexanoate (145 mg, 45% yield). ESI-MS m/z: 444 ([M+H]⁺).

General method for 34: To a 10 mL round-bottle flask was added a solution of 33 (1eq) in formic acid. Then the mixture was stirred at room temperature for overnight. Upon completion, the mixture was condensed and purified by flash column chromatography using a gradient of 0-4% MeOH/DCM to give target compounds 34.

3-[[2-(2,6-dioxo-3-piperidyl)-1,3-dioxo-isoindolin-4-yl]amino]propanoic acid (34a (n=2)): A yellow solid (43 mg, 71% yield). ESI-MS m/z: 346 ([M+H]⁺). ¹H-NMR (300 MHz, DMSO-d6): 11.1 (s, 1H), 7.59 (t, J=7.7 Hz, 1H), 7.15 (d, J=8.4 Hz, 1H), 7.04 (d, J=7.7 Hz, 1H), 6.66 (t, J=6.1 Hz, 1H), 5.04 (m, 1H), 3.52 (t, J=6.9 Hz, 2H), 2.87 (m, 1H), 2.57 (m, 2H), 2.56 (t, J=6.9 Hz, 2H), 2.02 (m, 1H).

4-[[2-(2,6-dioxo-3-piperidyl)-1,3-dioxo-isoindolin-4-yl]amino]butanoic acid (34b (n=3)): A yellow solid (40 mg, 66% yield). ESI-MS m/z: 360 ([M+H]⁺). ¹H-NMR (300 MHz, CD₂Cl₂): 8.45 (s, 1H), 7.53 (t, J=8.0 Hz, 1H), 7.08 (d, J=7.4 Hz, 1H), 6.96 (d, J=7.4 Hz, 1H), 4.92 (m, 1H), 3.37 (q, J=6.6 Hz, 2H), 2.82 (m, 1H), 2.76 (m, 2H), 2.49 (t, J=7.2 Hz, 2H), 2.12 (m, 1H), 1.99 (m, 2H).

5-[[2-(2,6-dioxo-3-piperidyl)-1,3-dioxo-isoindolin-4-yl]amino]pentanoic acid (34c (n=4)): A yellow solid (56 mg, 45% yield). ESI-MS m/z: 374 ([M+H]⁺). ¹H-NMR (300 MHz, CD₂Cl₂): 8.77 (s, 1H), 7.51 (t, J=8.1 Hz, 1H), 7.06 (d, J=7.3 Hz, 1H), 6.92 (d, J=7.4 Hz, 1H), 6.27 (m, 1H), 4.92 (m, 1H), 3.31 (q, J=6.6 Hz, 2H), 2.82 (m, 1H), 2.76 (m, 2H), 2.41 (t, J=7.2 Hz, 2H), 2.12 (m, 1H), 1.73 (m, 4H).

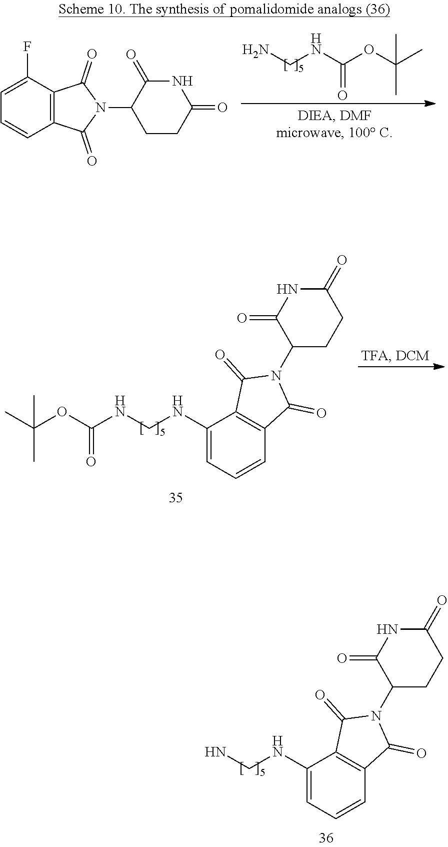

tert-butyl N-[5-[[2-(2,6-dioxo-3-piperidyl)-1,3-dioxo-isoindolin-4-yl]amino]pentyl]carbamate (35): To a 10 mL round-bottle flask was added a solution of 2-(2,6-dioxo-3-piperidyl)-4-fluoro-isoindoline-1,3-dione (0.72 mmol, 200 mg) and tert-butyl N-(5-aminopentyl) carbamate (0.72 mmol, 146 mg) in DMF (2 mL). Then DIEA (2.2 mmol, 372 μL) was added and the mixture was microwaved at 100° C. for 2 h. Upon completion, the mixture was condensed and purified by flash column chromatography using a gradient of 0-2% MeOH/DCM to give tert-butyl N-[5-[[2-(2,6-dioxo-3-piperidyl)-1,3-dioxo-isoindolin-4-yl]amino]pentyl]carbamate (120 mg, 36% yield). ESI-MS m/z: 459 ([M+H]⁺).

4-(5-aminopentylamino)-2-(2,6-dioxo-3-piperidyl) isoindoline-1,3-dione (36): To a 10 mL round-bottle flask was added a solution of tert-butyl N-[5-[[2-(2,6-dioxo-3-piperidyl)-1,3-dioxo-isoindolin-4-yl]amino]pentyl]carbamate (0.26 mmol, 120 mg) in formic acid (2 mL). Then the mixture was stirred at room temperature for overnight. Upon completion, the mixture was condensed and purified by flash column chromatography using a gradient of 0-4% MeOH/DCM to give 4-(5-aminopentylamino)-2-(2,6-dioxo-3-piperidyl) isoindoline-1,3-dione (85 mg, 91% yield). ESI-MS m/z: 359 ([M+H]⁺). ¹H-NMR (300 MHz, DMSO-d6): 11.1 (s, 1H), 7.58 (t, J=7.3 Hz, 1H), 7.09 (d, J=8.7 Hz, 1H), 7.03 (d, J=8.7 Hz, 1H), 6.54 (m, 1H), 5.05 (m, 1H), 3.29 (m, 2H), 2.85 (m, 1H), 2.79 (m, 2H), 2.55 (m, 2H), 2.03 (m, 1H), 1.58 (m, 4H), 1.39 (m, 2H).

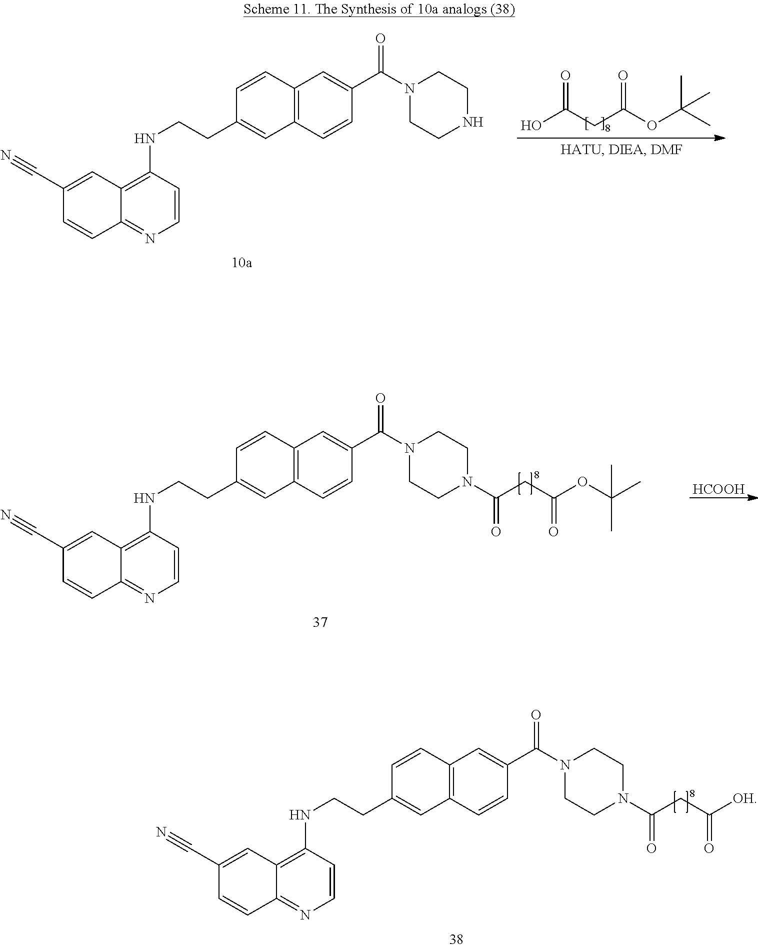

tert-butyl 10-[4-[6-[2-[(6-cyano-4-quinolyl)amino]ethyl]naphthalene-2-carbonyl]piperazin-1-yl]-10-oxo-decanoate (37): To a 10 mL round-bottle flask was added a solution of 10-tert-butoxy-10-oxo-decanoic acid (0.47 mmol, 121 mg) in DCM (5 mL). Then HATU (0.70 mmol, 266 mg), DIEA (1.40 mmol, 240 μL) were added and the mixture was stirred at room temperature for 15 min, then 4-[2-[6-(piperazine-1-carbonyl)-2-naphthyl]ethylamino]quinoline-6-carbonitrile (0.47 mmol, 203 mg) was added and the mixture was stirred for 2 h. Upon completion, water was added, the mixture was extracted with DCM, the organic layers were combined, washed with sat NaHCO₃ aq, dried by Na₂SO₄, condensed and purified by flash column chromatography using a gradient of 0-5% MeOH/DCM to give tert-butyl 10-[4-[6-[2-[(6-cyano-4-quinolyl)amino]ethyl]naphthalene-2-carbonyl]piperazin-1-yl]-10-oxo-decanoate (73 mg, 23% yield). ESI-MS m/z: 676 ([M+H]⁺). ¹H-NMR (300 MHz, CD₂Cl₂): 8.54 (d, J=5.7 Hz, 1H), 8.27 (s, 1H), 7.95 (d, J=9.0 Hz, 1H), 7.88 (s, 1H), 7.83 (t, J=7.9 Hz, 2H), 7.75 (dd, J=8.8, 1.7 Hz, 1H), 7.67 (s, 1H), 7.48 (dd, J=8.8, 1.7 Hz, 1H), 7.42 (dd, J=8.8, 1.7 Hz, 1H), 6.61 (d, J=5.7 Hz, 1H), 6.11 (m, 1H), 4.60 (m, 1H), 3.68 (m, 2H), 3.57 (m, 8H), 3.20 (t, J=7.6 Hz, 2H), 2.33 (m, 2H), 2.17 (t, J=8.4 Hz, 2H), 1.58 (m, 4H), 1.42 (s, 9H), 1.30 (m, 8H).

10-[4-[6-[2-[(6-cyano-4-quinolyl)amino]ethyl]naphthalene-2-carbonyl]piperazin-1-yl]-10-oxo-decanoic acid (38): To a 10 mL round-bottle flask was added a solution of tert-butyl 10-[4-[6-[2-[(6-cyano-4-quinolyl)amino]ethyl]naphthalene-2-carbonyl]piperazin-1-yl]-10-oxo-decanoate (0.11 mmol, 73 mg) in formic acid (2 mL). Then the mixture was stirred at room temperature for overnight. Upon completion, the mixture was condensed to give 10-[4-[6-[2-[(6-cyano-4-quinolyl)amino]ethyl]naphthalene-2-carbonyl]piperazin-1-yl]-10-oxo-decanoic acid (66 mg. 98% yield) and used without further purification. ESI-MS m/z: 620 ([M+H]⁺).

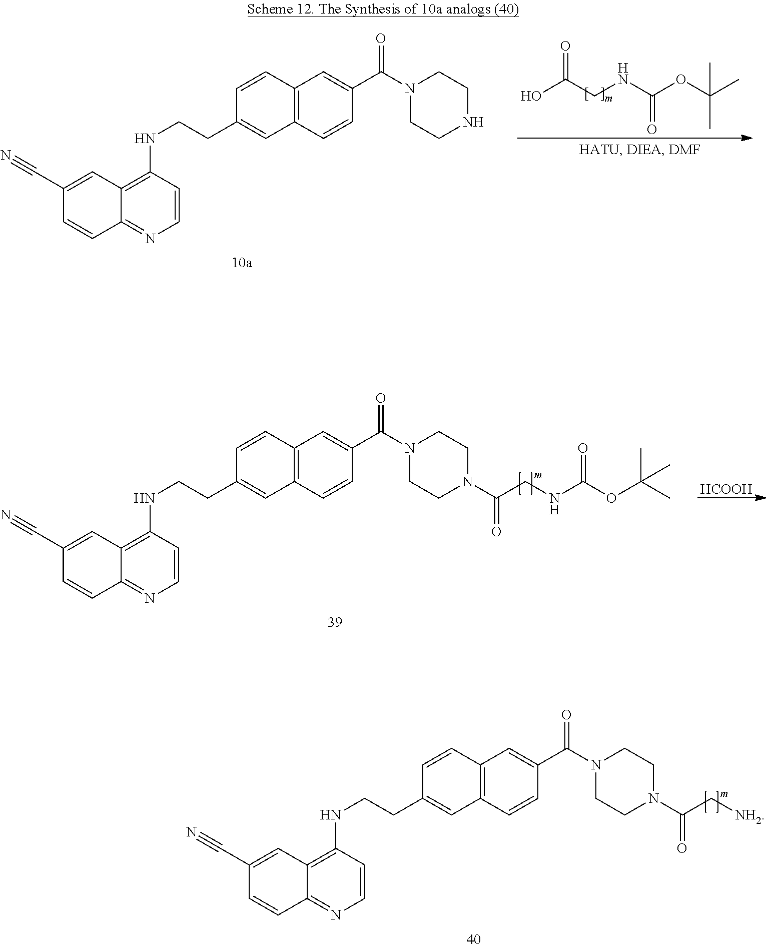

General method for 39: To a 10 mL round-bottle flask was added a solution of acid analogs (1eq) in DCM. Then HATU (1.5 eq), DIEA (3 eq) were added and the mixture was stirred at room temperature for 15 min, then 10a (1eq) was added and the mixture was stirred for 4 h. Upon completion, water was added, the mixture was extracted with DCM, the organic layers were combined, washed with sat NaHCO₃ aq, dried by Na₂SO₄, condensed and purified by flash column chromatography using a gradient of 0-5% MeOH/DCM to give target compounds 39.

tert-butyl N-[9-[4-[6-[2-[(6-cyano-4-quinolyl)amino]ethyl]naphthalene-2-carbonyl]piperazin-1-yl]-9-oxo-nonyl]carbamate (39a (m=8)): A light yellow solid (48 mg, 15% yield). ESI-MS m/z: 691 ([M+H]⁺). ¹H-NMR (300 MHz, CD₂Cl₂): 8.60 (d, J=5.7 Hz, 1H), 8.17 (s, 1H), 7.97 (d, J=9.3 Hz, 1H), 7.89 (s, 2H), 7.83 (t, J=9.3 Hz, 1H), 7.73 (dd, J=8.8, 1.7 Hz, 1H), 7.71 (s, 1H), 7.49 (dd, J=8.8, 1.7 Hz, 1H), 7.45 (dd, J=8.8, 1.7 Hz, 1H), 6.60 (d, J=5.7 Hz, 1H), 5.63 (m, 1H), 4.60 (m, 1H), 3.70 (q, J=6.6 Hz, 2H), 3.57 (m, 8H), 3.22 (t, J=7.4 Hz, 2H), 3.05 (q, J=6.6 Hz, 2H), 2.32 (m, 2H), 1.59 (m, 2H), 1.41 (s, 9H), 1.30 (m, 10H).

tert-butyl N-[10-[4-[6-[2-[(6-cyano-4-quinolyl)amino]ethyl]naphthalene-2-carbonyl]piperazin-1-yl]-10-oxo-decyl]carbamate (39b (m=9)): A light yellow solid (89 mg, 37% yield). ESI-MS m/z: 705 ([M+H]⁺). ¹H-NMR (300 MHz, CD₂Cl₂): 8.56 (d, J=5.6 Hz, 1H), 8.26 (s, 1H), 7.95 (d, J=8.6 Hz, 1H), 7.88 (s, 1H), 7.83 (d, J=7.7 Hz, 2H), 7.73 (dd, J=8.8, 1.7 Hz, 1H), 7.68 (s, 1H), 7.48 (dd, J=8.8, 1.7 Hz, 1H), 7.42 (dd, J=8.8, 1.7 Hz, 1H), 6.60 (d, J=5.6 Hz, 1H), 6.02 (m, 1H), 4.62 (m, 1H), 3.67 (q, J=7.3 Hz, 2H), 3.56 (m, 8H), 3.19 (t, J=7.6 Hz, 2H), 3.05 (q, J=6.8 Hz, 2H), 2.32 (m, 2H), 1.59 (m, 2H), 1.41 (s, 12H), 1.27 (m, 9H).

tert-butyl N-[12-[4-[6-[2-[(6-cyano-4-quinolyl)amino]ethyl]naphthalene-2-carbonyl]piperazin-1-yl]-12-oxo-dodecyl]carbamate (39c (m=10)): A light yellow solid (44 mg, 13% yield). ESI-MS m/z: 733 ([M+H]⁺). ¹H-NMR (300 MHz, CD₂Cl₂): 8.60 (d, J=5.6 Hz, 1H), 8.16 (s, 1H), 7.97 (d, J=8.6 Hz, 1H), 7.89 (s, 2H), 7.84 (d, J=8.6 Hz, 1H), 7.73 (dd, J=8.8, 1.7 Hz, 1H), 7.71 (s, 1H), 7.49 (dd, J=8.8, 1.7 Hz, 1H), 7.45 (dd, J=8.8, 1.7 Hz, 1H), 6.60 (d, J=5.6 Hz, 1H), 5.63 (m, 1H), 4.60 (m, 1H), 3.71 (q, J=7.3 Hz, 2H), 3.57 (m, 8H), 3.22 (t, J=7.6 Hz, 2H), 3.05 (q, J=6.8 Hz, 2H), 2.32 (m, 2H), 2.00 (m, 2H), 1.59 (m, 2H), 1.41 (s, 9H), 1.27 (m, 12H).

tert-butyl N-[12-[4-[6-[2-[(6-cyano-4-quinolyl)amino]ethyl]naphthalene-2-carbonyl]piperazin-1-yl]-12-oxo-dodecyl]carbamate (39d (m=11)): A light yellow solid (36 mg, 11% yield). ESI-MS m/z: 733 ([M+H]⁺). ¹H-NMR (300 MHz, CD₂Cl₂): 8.60 (d, J=5.6 Hz, 1H), 8.17 (s, 1H), 7.97 (d, J=8.6 Hz, 1H), 7.88 (s, 2H), 7.83 (d, J=8.6 Hz, 1H), 7.73 (dd, J=8.8, 1.7 Hz, 1H), 7.72 (s, 1H), 7.49 (dd, J=8.8, 1.7 Hz, 1H), 7.45 (dd, J=8.8, 1.7 Hz, 1H), 6.60 (d, J=5.6 Hz, 1H), 5.65 (m, 1H), 4.59 (m, 1H), 3.71 (q, J=7.3 Hz, 2H), 3.58 (m, 8H), 3.22 (t, J=7.6 Hz, 2H), 3.05 (q, J=6.8 Hz, 2H), 2.32 (m, 2H), 1.86 (m, 4H), 1.59 (m, 2H), 1.41 (s, 9H), 1.27 (m, 12H).

General method for 40: To a 10 mL round-bottle flask was added a solution of 39 (1eq) in formic acid. The mixture was stirred at room temperature for overnight. Upon completion, the mixture was condensed to give target compounds 40 and used without further purification.

4-[2-[6-[4-(9-aminononanoyl) piperazine-1-carbonyl]-2-naphthyl]ethylamino]quinoline-6-carbonitrile (40a (m=8)): A white solid (40 mg, 98% yield) and used without further purification. ESI-MS m/z: 591 ([M+H]⁺).

4-[2-[6-[4-(10-aminodecanoyl) piperazine-1-carbonyl]-2-naphthyl]ethylamino]quinoline-6-carbonitrile (40b (m=9)): A white solid (67 mg, 98% yield) and used without further purification. ESI-MS m/z: 605 ([M+H]⁺).

4-[2-[6-[4-(12-aminododecanoyl) piperazine-1-carbonyl]-2-naphthyl]ethylamino]quinoline-6-carbonitrile (40c (m=10)): A white solid (38 mg, 98% yield) and used without further purification. ESI-MS m/z: 633 ([M+H]⁺).

4-[2-[6-[4-(12-aminododecanoyl) piperazine-1-carbonyl]-2-naphthyl]ethylamino]quinoline-6-carbonitrile (40d (m=11)): A white solid (31 mg, 98% yield) and used without further purification. ESI-MS m/z: 633 ([M+H]⁺).

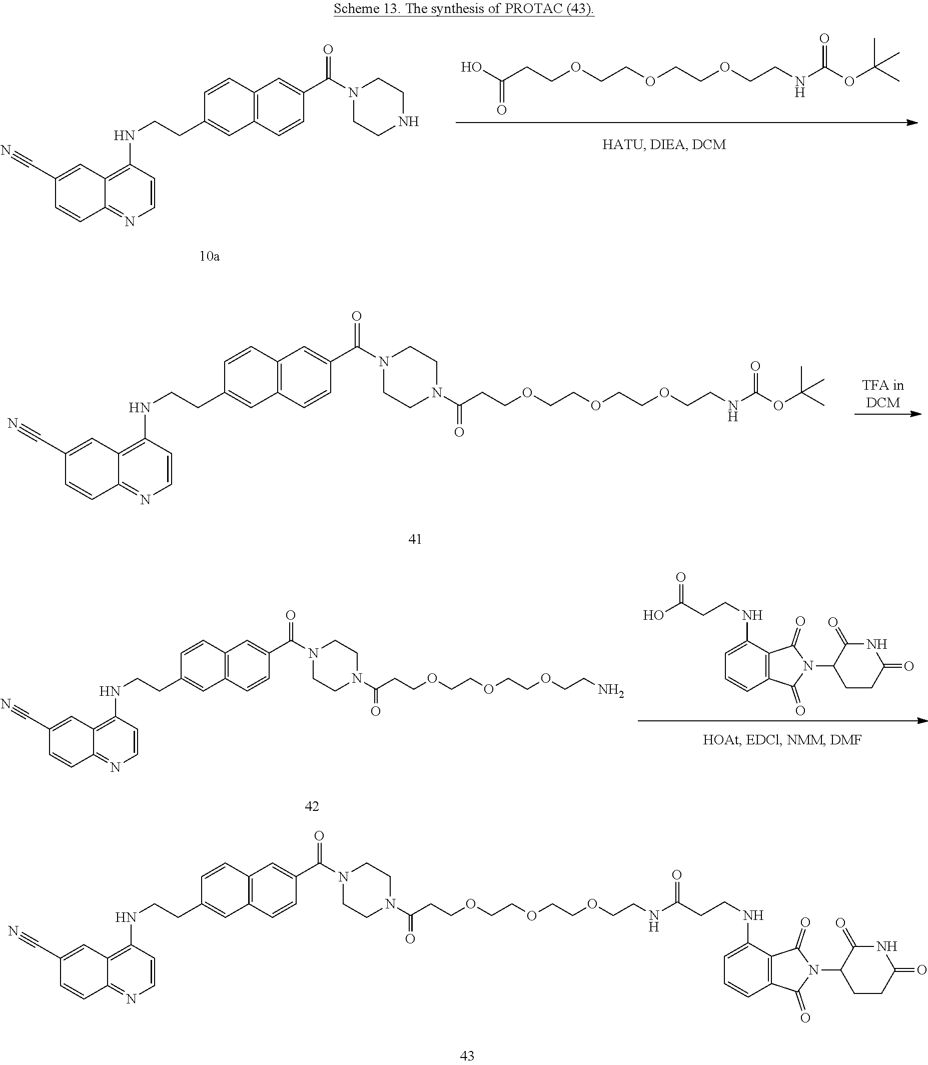 [SMILES: N#Cc1ccc2nccc(NCCc3ccc4cc(C(=O)N5CCNCC5)ccc4c3)c2c1 ; CC(C)(C)OC(=O)NCCOCCOCCOCCC(=O)O ; CC(C)(C)OC(=O)NCCOCCOCCOCCC(=O)N1CCN(C(=O)c2ccc3cc(CCNc4ccnc5ccc(C#N)cc45)ccc3c2)CC1 ; N#Cc1ccc2nccc(NCCc3ccc4cc(C(=O)N5CCN(C(=O)CCOCCOCCOCCN)CC5)ccc4c3)c2c1 ; O=C(O)CCNc1cccc2c1C(=O)N(C1CCC(=O)NC1=O)C2=O ; N#Cc1ccc2nccc(NCCc3ccc4cc(C(=O)N5CCN(C(=O)CCOCCOCCOCCNC(=O)CCNc6cccc7c6C(=O)N(C6CCC(=O)NC6=O)C7=O)CC5)ccc4c3)c2c1]

tert-butyl N-[2-[2-[2-[3-[4-[6-[2-[(6-cyano-4-quinolyl)amino]ethyl]naphthalene-2-carbonyl]piperazin-1-yl]-3-oxo-propoxy]ethoxy]ethoxy]ethyl]carbamate (41): To a 10 mL round-bottle flask was added a solution of 3-[2-[2-[2-(tert-butoxycarbonylamino) ethoxy]ethoxy]ethoxy]propanoic acid (0.47 mmol, 150 mg) in DCM (5 mL). Then HATU (0.61 mmol, 230 mg), DIEA (1.40 mmol, 240 μL) were added and the mixture was stirred at room temperature for 15 min, then 4-[2-[6-(piperazine-1-carbonyl)-2-naphthyl]ethylamino]quinoline-6-carbonitrile (0.47 mmol, 203 mg) was added and the mixture was stirred for 2 h. Upon completion, water was added, the mixture was extracted with DCM, the organic layers were combined, washed with sat NaHCO₃ aq, dried by Na₂SO₄, condensed and purified by flash column chromatography using a gradient of 0-5% MeOH/DCM to give tert-butyl N-[2-[2-[2-[3-[4-[6-[2-[(6-cyano-4-quinolyl)amino]ethyl]naphthalene-2-carbonyl]piperazin-1-yl]-3-oxo-propoxy]ethoxy]ethoxy]ethyl]carbamate (82 mg, 24% yield). ESI-MS m/z: 739 ([M+H]⁺). ¹H-NMR (300 MHz, CD₂Cl₂): 8.61 (d, J=5.7 Hz, 1H), 8.17 (s, 1H), 7.99 (d, J=9.3 Hz, 1H), 7.89 (s, 2H), 7.83 (d, J=9.3 Hz, 1H), 7.77 (d, J=7.5 Hz, 2H), 7.74 (dd, J=8.8, 1.7 Hz, 1H), 7.71 (s, 1H), 7.49 (dd, J=8.8, 1.7 Hz, 1H), 7.45 (dd, J=8.8, 1.7 Hz, 1H), 6.61 (d, J=5.7 Hz, 1H), 5.69 (m, 1H), 5.11 (m, 1H), 3.73 (m, 8H), 3.57 (m, 12H), 3.47 (m, 2H), 3.22 (m, 4H), 2.63 (m, 2H), 1.40 (s, 9H).

tert-butyl N-[2-[2-[2-[3-[4-[6-[2-[(6-cyano-4-quinolyl)amino]ethyl]naphthalene-2-carbonyl]piperazin-1-yl]-3-oxo-propoxy]ethoxy]ethoxy]ethyl]carbamate (42): To a 10 mL round-bottle flask was added a solution of tert-butyl N-[2-[2-[2-[3-[4-[6-[2-[(6-cyano-4-quinolyl)amino]ethyl]naphthalene-2-carbonyl]piperazin-1-yl]-3-oxo-propoxy]ethoxy]ethoxy]ethyl]carbamate (0.11 mmol, 82 mg) in DCM (5 mL). Then TFA (1 mL) was added and the mixture was stirred at room temperature for 4 h. Upon completion, the mixture was condensed to give 4-[2-[6-[4-[3-[2-[2-(2-aminoethoxy) ethoxy]ethoxy]propanoyl]piperazine-1-carbonyl]-2-naphthyl]ethylamino]quinoline-6-carbonitrile (70 mg, 99% yield) and used without further purification. ESI-MS m/z: 639 ([M+H]⁺).

N-[2-[2-[2-[3-[4-[6-[2-[(6-cyano-4-quinolyl)amino]ethyl]naphthalene-2-carbonyl]piperazin-1-yl]-3-oxo-propoxy]ethoxy]ethoxy]ethyl]-3-[[2-(2,6-dioxo-3-piperidyl)-1,3-dioxo-isoindolin-4-yl]amino]propanamide (43): To a 10 mL round-bottle flask was added a solution of 4-[2-[6-[4-[3-[2-[2-(2-aminoethoxy) ethoxy]ethoxy]propanoyl]piperazine-1-carbonyl]-2-naphthyl]ethylamino]quinoline-6-carbonitrile (0.11 mmol, 70 mg) and 3-[[2-(2,6-dioxo-3-piperidyl)-1,3-dioxo-isoindolin-4-yl]amino]propanoic acid (0.11 mmol, 38 mg) in DMF (2 mL). Then EDC-HCl (0.22 mmol, 42 mg), HOAt (0.22 mmol, 30 mg), and N-methylmorpholine (0.55 mmol, 55 mg) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give N-[2-[2-[2-[3-[4-[6-[2-[(6-cyano-4-quinolyl)amino]ethyl]naphthalene-2-carbonyl]piperazin-1-yl]-3-oxo-propoxy]ethoxy]ethoxy]ethyl]-3-[[2-(2,6-dioxo-3-piperidyl)-1,3-dioxo-isoindolin-4-yl]amino]propanamide (62 mg, 59% yield). ESI-MS m/z: 967 ([M+H]⁺). ¹H-NMR (300 MHz, CD₂Cl₂): 9.00 (m, 1H), 8.60 (d, J=5.7 Hz, 1H), 8.17 (s, 1H), 7.98 (d, J=8.9 Hz, 1H), 7.88 (s, 1H), 7.83 (t, J=8.3 Hz, 2H), 7.71 (d, J=8.9 Hz, 1H), 7.69 (s, 1H), 7.47 (d, J=8.4 Hz, 2H), 7.42 (t, J=8.3 Hz, 2H), 6.98 (d, J=7.2 Hz, 1H), 6.90 (d, J=7.2 Hz, 1H), 6.71 (m, 1H), 6.58 (d, J=5.6 Hz, 1H), 6.52 (t, J=6.1 Hz, 1H), 5.75 (m, 1H), 4.89 (m, 1H), 3.75 (t, J=6.7 Hz, 2H), 3.68 (t, J=5.9 Hz, 2H), 3.55 (m, 18H), 3.37 (m, 2H), 3.20 (t, J=7.5 Hz, 2H), 2.80 (m, 1H), 2.73 (m, 2H), 2.60 (m, 2H), 2.48 (t, J=6.4 Hz, 2H), 2.08 (m, 1H), 1.94 (m, 2H).

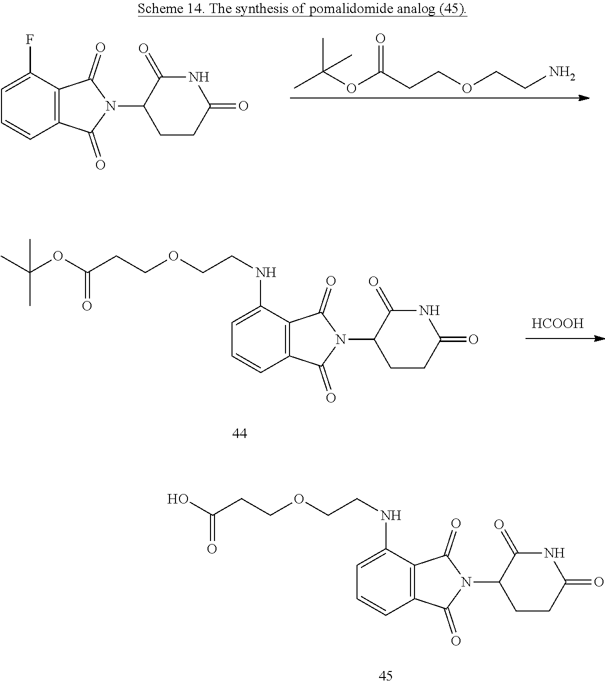 [SMILES: O=C1CCC(N2C(=O)c3cccc(F)c3C2=O)C(=O)N1 ; CC(C)(C)OC(=O)CCOCCN ; CC(C)(C)OC(=O)CCOCCNc1cccc2c1C(=O)N(C1CCC(=O)NC1=O)C2=O ; "HCOOH" ; O=C(O)CCOCCNc1cccc2c1C(=O)N(C1CCC(=O)NC1=O)C2=O]

tert-butyl 3-[2-[[2-(2,6-dioxo-3-piperidyl)-1,3-dioxo-isoindolin-4-yl]amino]ethoxy]propanoate (44): To a 10 mL round-bottle flask was added a solution of 2-(2,6-dioxo-3-piperidyl)-4-fluoro-isoindoline-1,3-dione (0.72 mmol, 200 mg) and tert-butyl 3-(2-aminoethoxy) propanoate (0.72 mmol, 137 mg) in DMF (2 mL). Then DIEA (1.1 mmol, 186 μL) was added and the mixture was microwaved at 100° C. for 2 h. Upon completion, the mixture was condensed and purified by flash column chromatography using a gradient of 0-2% MeOH/DCM to give tert-butyl 3-[2-[[2-(2,6-dioxo-3-piperidyl)-1,3-dioxo-isoindolin-4-yl]amino]ethoxy]propanoate (200 mg, 62% yield). ESI-MS m/z: 446 ([M+H]⁺).

3-[2-[[2-(2,6-dioxo-3-piperidyl)-1,3-dioxo-isoindolin-4-yl]amino]ethoxy]propanoic acid (45): To a 10 mL round-bottle flask was added a solution of tert-butyl 3-[2-[[2-(2,6-dioxo-3-piperidyl)-1,3-dioxo-isoindolin-4-yl]amino]ethoxy]propanoate (0.45 mmol, 200 mg) in formic acid (2 mL). Then the mixture was stirred at room temperature for overnight. Upon completion, the mixture was condensed and purified by flash column chromatography using a gradient of 0-4% MeOH/DCM to give 3-[2-[[2-(2,6-dioxo-3-piperidyl)-1,3-dioxo-isoindolin-4-yl]amino]ethoxy]propanoic acid (127 mg, 73% yield). ESI-MS m/z: 390 ([M+H]⁺). ¹H-NMR (300 MHz, CD₂Cl₂): 8.72 (m, 1H), 7.50 (t, J=8.1 Hz, 1H), 7.07 (d, J=7.3 Hz, 1H), 6.95 (d, J=8.1 Hz, 1H), 6.46 (m, 1H), 4.94 (m, 1H), 3.74 (t, J=6.1 Hz, 2H), 3.69 (t, J=5.6 Hz, 2H), 3.46 (m, 2H), 2.78 (m, 1H), 2.76 (t, J=10.7 Hz, 2H), 2.59 (t, J=6.1 Hz, 2H), 2.11 (m, 1H).

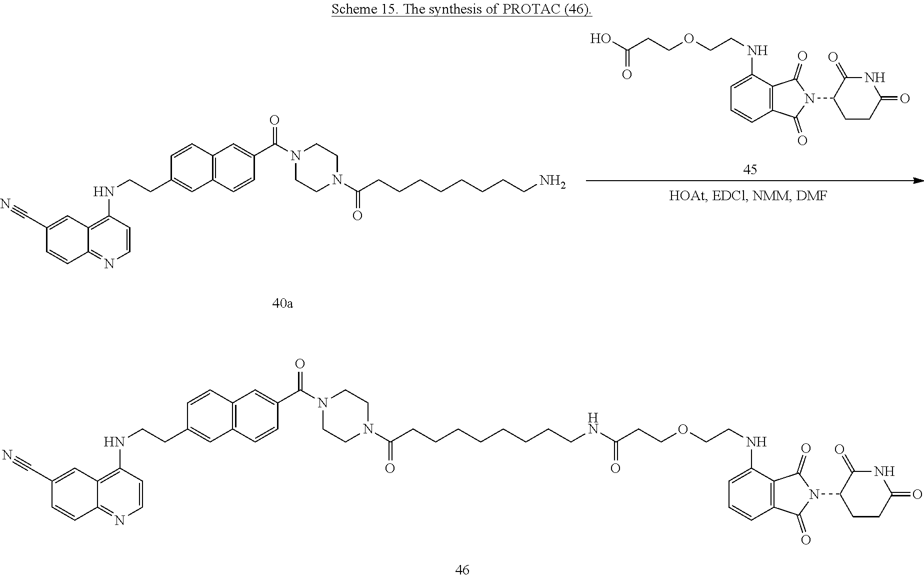

N-[9-[4-[6-[2-[(6-cyano-4-quinolyl)amino]ethyl]naphthalene-2-carbonyl]piperazin-1-yl]-9-oxo-nonyl]-3-[2-[[2-(2,6-dioxo-3-piperidyl)-1,3-dioxo-isoindolin-4-yl]amino]ethoxy]propanamide (46): To a 10 mL round-bottle flask was added a solution of 4-[2-[6-[4-(9-aminononanoyl) piperazine-1-carbonyl]-2-naphthyl]ethylamino]quinoline-6-carbonitrile (0.07 mmol, 40 mg) and 3-[2-[[2-(2,6-dioxo-3-piperidyl)-1,3-dioxo-isoindolin-4-yl]amino]ethoxy]propanoic acid (0.07 mmol, 26 mg) in DMF (2 mL). Then EDC-HCl (0.14 mmol, 26 mg), HOAt (0.14 mmol, 18 mg), and N-methylmorpholine (0.33 mmol, 34 mg) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give N-[9-[4-[6-[2-[(6-cyano-4-quinolyl)amino]ethyl]naphthalene-2-carbonyl]piperazin-1-yl]-9-oxo-nonyl]-3-[2-[[2-(2,6-dioxo-3-piperidyl)-1,3-dioxo-isoindolin-4-yl]amino]ethoxy]propanamide (52 mg, 80% yield). ESI-MS m/z: 963 ([M+H]⁺). ¹H-NMR (300 MHz, CD₂Cl₂): 9.22 (m, 1H), 8.59 (d, J=5.1 Hz, 1H), 8.24 (s, 1H), 7.99 (d, J=9.2 Hz, 1H), 7.87 (s, 1H), 7.84 (d, J=8.3 Hz, 1H), 7.81 (d, J=8.3 Hz, 1H), 7.70 (d, J=9.1 Hz, 1H), 7.69 (s, 1H), 7.49 (t, J=8.3 Hz, 2H), 7.42 (d, J=8.3 Hz, 1H), 7.05 (d, J=6.8 Hz, 1H), 6.92 (d, J=6.8 Hz, 1H), 6.57 (d, J=5.4 Hz, 1H), 6.47 (m, 1H), 6.15 (m, 1H), 6.00 (m, 1H), 4.91 (m, 1H), 3.72 (t, J=6.0 Hz, 2H), 3.67 (t, J=5.2 Hz, 2H), 3.56 (m, 6H), 3.44 (q, J=5.2 Hz, 2H), 3.19 (t, J=7.5 Hz, 2H), 3.12 (q, J=6.7 Hz, 2H), 2.77 (m, 3H), 2.40 (t, J=6.6 Hz, 2H), 2.30 (m, 2H), 2.24 (m, 2H), 2.10 (m, 1H), 1.55 (m, 2H), 1.38 (m, 2H), 1.22 (m, 10H).

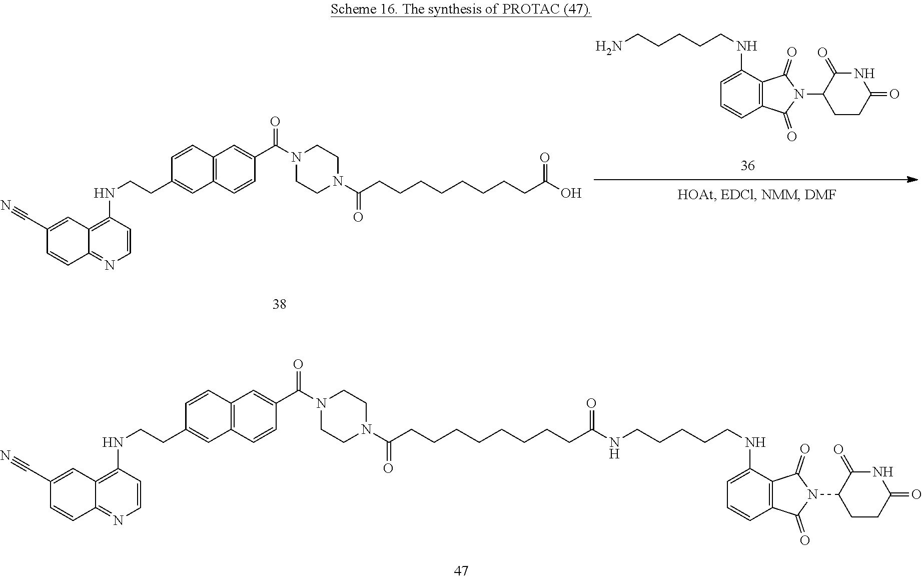

10-[4-[6-[2-[(6-cyano-4-quinolyl)amino]ethyl]naphthalene-2-carbonyl]piperazin-1-yl]-N-[5-[[2-(2,6-dioxo-3-piperidyl)-1,3-dioxo-isoindolin-4-yl]amino]pentyl]-10-oxo-decanamide (47): To a 10 mL round-bottle flask was added a solution of 10-[4-[6-[2-[(6-cyano-4-quinolyl)amino]ethyl]naphthalene-2-carbonyl]piperazin-1-yl]-10-oxo-decanoic acid (0.11 mmol, 66 mg) and 6-[[2-(2,6-dioxo-3-piperidyl)-1,3-dioxo-isoindolin-4-yl]amino]hexanoic acid (0.11 mmol, 42 mg) in DMF (2 mL). Then EDC-HCl (0.21 mmol, 41 mg), HOAt (0.21 mmol, 29 mg), and N-methylmorpholine (0.53 mmol, 54 mg) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give 10-[4-[6-[2-[(6-cyano-4-quinolyl)amino]ethyl]naphthalene-2-carbonyl]piperazin-1-yl]-N-[5-[[2-(2,6-dioxo-3-piperidyl)-1,3-dioxo-isoindolin-4-yl]amino]pentyl]-10-oxo-decanamide (35 mg, 34% yield). ESI-MS m/z: 961 ([M+H]⁺). ¹H-NMR (300 MHz, CD₂Cl₂): 8.99 (m, 1H), 8.60 (d, J=5.7 Hz, 1H), 8.21 (s, 1H), 7.98 (d, J=8.9 Hz, 1H), 7.88 (s, 1H), 7.83 (t, J=8.3 Hz, 2H), 7.71 (d, J=8.9 Hz, 1H), 7.69 (s, 1H), 7.48 (d, J=8.4 Hz, 2H), 7.45 (t, J=8.3 Hz, 2H), 7.02 (d, J=7.2 Hz, 1H), 6.88 (d, J=8.2 Hz, 1H), 6.57 (d, J=5.5 Hz, 1H), 6.22 (t, J=5.5 Hz, 1H), 5.86 (t, J=5.0 Hz, 1H), 5.72 (t, J=5.5 Hz, 1H), 4.89 (m, 1H), 3.59 (m, 8H), 3.21 (m, 6H), 2.80 (m, 1H), 2.74 (m, 2H), 2.31 (m, 2H), 2.11 (m, 1H), 2.10 (t, J=7.4 Hz, 2H), 1.88 (m, 2H), 1.55 (m, 8H), 1.27 (s, 10H).

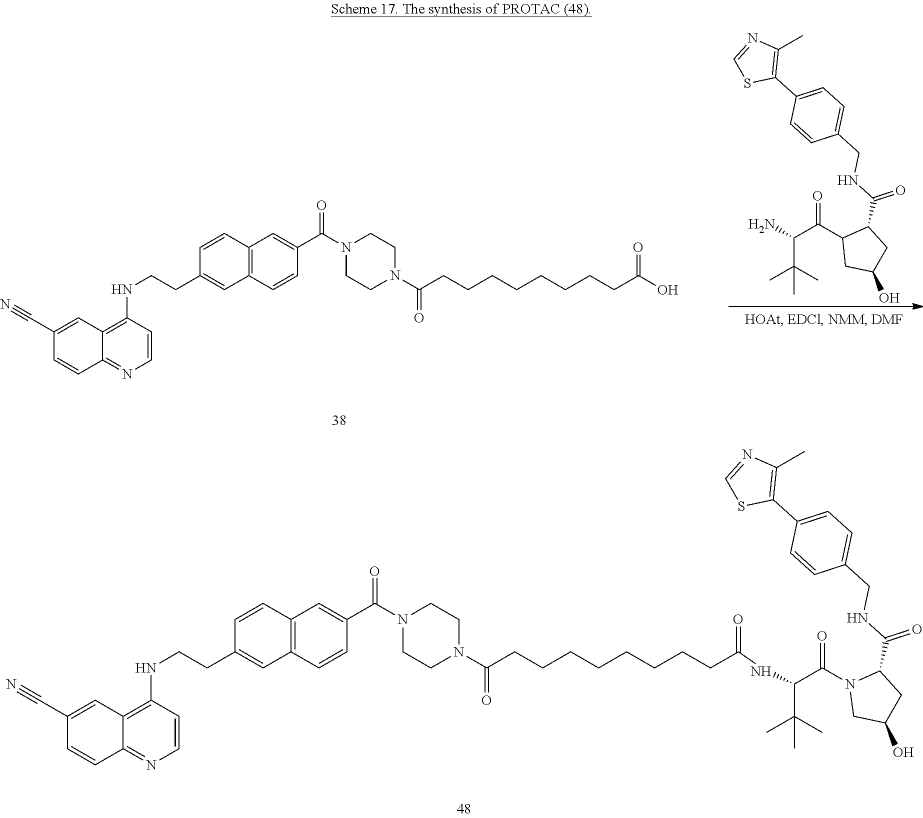 [SMILES: N#Cc1ccc2nccc(NCCc3ccc4cc(C(=O)N5CCN(C(=O)CCCCCCCCC(=O)O)CC5)ccc4c3)c2c1 ; Cc1ncsc1-c1ccc(CNC(=O)[C@@H]2C[C@@H](O)CC2C(=O)[C@@H](N)C(C)(C)C)cc1 ; Cc1ncsc1-c1ccc(CNC(=O)[C@@H]2C[C@@H](O)CN2C(=O)[C@@H](NC(=O)CCCCCCCCC(=O)N2CCN(C(=O)c3ccc4cc(CCNc5ccnc6ccc(C#N)cc56)ccc4c3)CC2)C(C)(C)C)cc1]

rac-(2S,4R)-4-hydroxy-N-[[4-(4-methylthiazol-5-yl)phenyl]methyl]-1-[rac-(2S)-2-[[10-[4-[6-[2-[(6-cyano-4-quinolyl)amino]ethyl]naphthalene-2-carbonyl]piperazin-1-yl]-10-oxo-decanoyl]amino]-3,3-dimethyl-butanoyl]pyrrolidine-2-carboxamide (48): To a 10 mL round-bottle flask was added a solution of 10-[4-[6-[2-[(6-cyano-4-quinolyl)amino]ethyl]naphthalene-2-carbonyl]piperazin-1-yl]-10-oxo-decanoic acid (0.13 mmol, 80 mg) and 5-[[2-(2,6-dioxo-3-piperidyl)-1,3-dioxo-isoindolin-4-yl]amino]pentanoic acid (0.13 mmol, 49 mg) in DMF (2 mL). Then EDC-HCl (0.27 mmol, 51 mg), HOAt (0.27 mmol, 36 mg), and N-methylmorpholine (0.27 mmol, 67 mg) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give N-[10-[4-[6-[2-[(6-cyano-4-quinolyl)amino]ethyl]naphthalene-2-carbonyl]piperazin-1-yl]-10-oxo-decyl]-5-[[2-(2,6-dioxo-3-piperidyl)-1,3-dioxo-isoindolin-4-yl]amino]pentanamide (73 mg, 61% yield). ESI-MS m/z: 1033 ([M+H]⁺). ¹H-NMR (300 MHz, CD₂Cl₂): 8.63 (s, 1H), 8.57 (d, J=5.5 Hz, 1H), 8.30 (s, 1H), 7.96 (d, J=9.3 Hz, 1H), 7.86 (s, 1H), 7.81 (t, J=8.8 Hz, 2H), 7.71 (dd, J=8.8, 1.5 Hz, 1H), 7.67 (s, 1H), 7.43 (m, 3H), 7.34 (m, 5H), 6.59 (d, J=6.0 Hz, 1H), 6.32 (d, J=9.3 Hz, 1H), 6.28 (m, 1H), 4.64 (m, 1H), 4.51 (m, 2H), 4.28 (m, 1H), 3.97 (m, 1H), 3.70-3.53 (m, 12H), 3.19 (t, J=6.8 Hz, 2H), 2.44 (m, 2H), 2.32 (m, 2H), 2.15 (m, 2H), 2.12 (s, 3H), 1.56 (m, 4H), 1.25 (m, 8H), 0.94 (s, 9H).

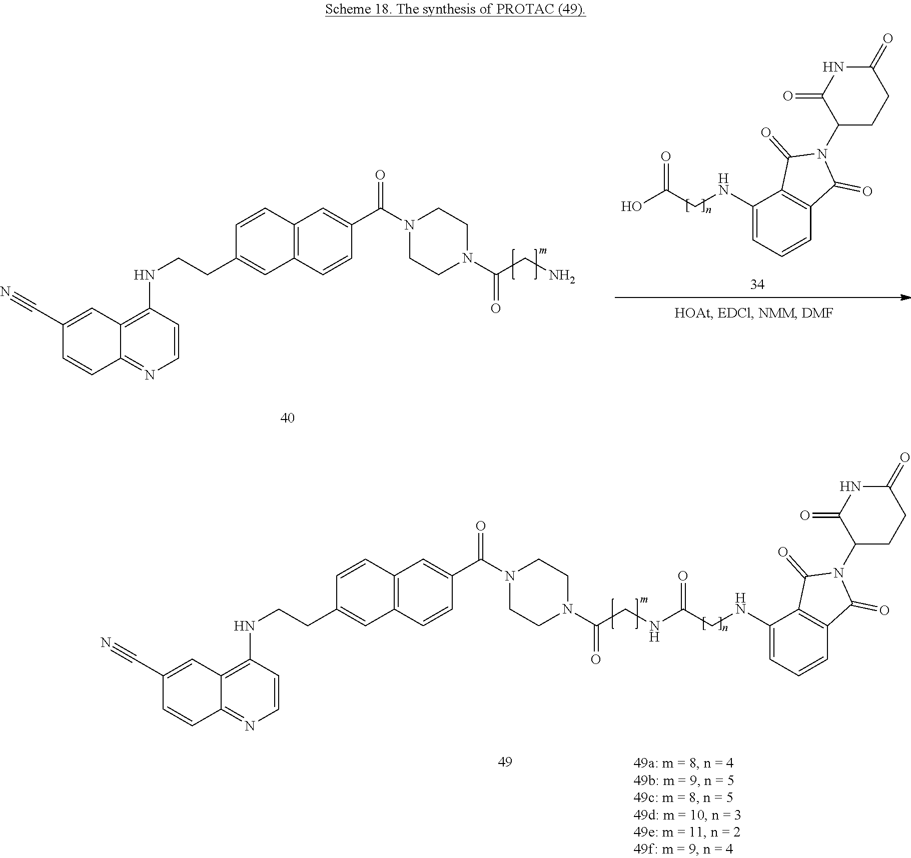

General method for 49: To a 10 mL round-bottle flask was added a solution of 40 (1eq) and 34 (1eq) in DMF. Then EDC-HCl (2 eq), HOAt (2 eq), and N-methylmorpholine (5 eq) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give target compounds 49.

N-[9-[4-[6-[2-[(6-cyano-4-quinolyl)amino]ethyl]naphthalene-2-carbonyl]piperazin-1-yl]-9-oxo-nonyl]-5-[[2-(2,6-dioxo-3-piperidyl)-1,3-dioxo-isoindolin-4-yl]amino]pentanamide (49a (m=8, n=4)): A yellow solid (33 mg, 25% yield). ESI-MS m/z: 947 ([M+H]⁺). ¹H-NMR (300 MHz, DMSO-d6): 11.10 (s, 1H), 8.86 (s, 1H), 8.52 (d, J=5.3 Hz, 1H), 7.97 (s, 1H), 7.93 (d, J=5.3 Hz, 1H), 7.89 (d, J=5.3 Hz, 1H), 7.88 (s, 3H), 7.76 (t, J=5.7 Hz, 1H), 7.70 (t, J=5.7 Hz, 1H), 7.54 (m, 3H), 7.07 (d, J=8.7 Hz, 1H), 7.01 (d, J=7.0 Hz, 1H), 6.71 (d, J=5.7 Hz, 1H), 6.56 (t, J=5.4 Hz, 1H), 5.05 (m, 1H), 3.65 (q, J=7.3 Hz, 2H), 3.50 (m, 6H), 3.28 (m, 2H), 3.18 (t, J=7.3 Hz, 2H), 3.00 (q, J=6.4 Hz, 2H), 2.88 (m, 1H), 2.57 (m, 2H), 2.29 (m, 2H), 2.08 (m, 2H), 2.00 (m, 1H), 1.54 (m, 4H), 1.46 (m, 2H), 1.35 (m, 2H), 1.22 (s, 10H).

N-[10-[4-[6-[2-[(6-cyano-4-quinolyl)amino]ethyl]naphthalene-2-carbonyl]piperazin-1-yl]-10-oxo-decyl]-6-[[2-(2,6-dioxo-3-piperidyl)-1,3-dioxo-isoindolin-4-yl]amino]hexanamide (49b (m=9, n=5)): A yellow solid (80 mg, 74% yield). ESI-MS m/z: 975 ([M+H]⁺). ¹H-NMR (300 MHz, DMSO-d6): 11.11 (s, 1H), 8.86 (s, 1H), 8.52 (d, J=5.3 Hz, 1H), 7.97 (s, 1H), 7.93 (d, J=5.3 Hz, 1H), 7.89 (d, J=5.3 Hz, 1H), 7.88 (s, 3H), 7.71 (m, 2H), 7.54 (m, 3H), 7.06 (d, J=8.7 Hz, 1H), 7.00 (d, J=7.0 Hz, 1H), 6.71 (d, J=5.7 Hz, 1H), 6.51 (t, J=5.4 Hz, 1H), 5.05 (m, 1H), 3.65 (q, J=6.4 Hz, 2H), 3.51 (m, 6H), 3.27 (q, J=6.4 Hz, 2H), 3.18 (t, J=7.3 Hz, 2H), 2.99 (q, J=6.4 Hz, 2H), 2.88 (m, 1H), 2.57 (m, 2H), 2.29 (m, 2H), 2.04 (m, 2H), 2.00 (m, 1H), 1.51 (m, 6H), 1.32 (m, 4H), 1.22 (s, 12H).

N-[9-[4-[6-[2-[(6-cyano-4-quinolyl)amino]ethyl]naphthalene-2-carbonyl]piperazin-1-yl]-9-oxo-nonyl]-6-[[2-(2,6-dioxo-3-piperidyl)-1,3-dioxo-isoindolin-4-yl]amino]hexanamide (49c (m=8, n=5)): A yellow solid (51 mg, 31% yield). ESI-MS m/z: 961 ([M+H]⁺). ¹H-NMR (300 MHz, DMSO-d6): 11.11 (s, 1H), 8.86 (s, 1H), 8.52 (d, J=5.7 Hz, 1H), 7.97 (s, 1H), 7.93 (d. J=5.3 Hz, 1H), 7.89 (d, J=5.3 Hz, 1H), 7.88 (s, 3H), 7.71 (m, 2H), 7.54 (m, 3H), 7.06 (d, J=8.7 Hz, 1H), 7.00 (d, J=7.0 Hz, 1H), 6.71 (d, J=5.7 Hz, 1H), 6.51 (t, J=5.4 Hz, 1H), 5.05 (m, 1H), 3.65 (q, J=6.4 Hz, 2H), 3.51 (m, 6H), 3.26 (q, J=6.4 Hz, 2H), 3.18 (t, J=7.3 Hz, 2H), 2.99 (q, J=6.4 Hz, 2H), 2.86 (m, 1H), 2.57 (m, 2H), 2.29 (m, 2H), 2.05 (m, 2H), 1.51 (m, 6H), 1.32 (m, 4H), 1.22 (s, 10H).

N-[12-[4-[6-[2-[(6-cyano-4-quinolyl)amino]ethyl]naphthalene-2-carbonyl]piperazin-1-yl]-12-oxo-dodecyl]-3-[[2-(2,6-dioxo-3-piperidyl)-1,3-dioxo-isoindolin-4-yl]amino]propanamide (49d (m=10, n=3)): A yellow solid (44 mg, 75% yield). ESI-MS m/z: 961 ([M+H]⁺). ¹H-NMR (300 MHz, DMSO-d6): 11.10 (s, 1H), 8.93 (s, 1H), 8.53 (d, J=5.9 Hz, 1H), 8.10 (m, 1H), 7.97 (s, 2H), 7.94 (s, 1H), 7.92 (s, 1H), 7.89 (s, 2H), 7.81 (t, J=5.4 Hz, 1H), 7.58 (d, J=7.5 Hz, 2H), 7.52 (t, J=7.0 Hz, 1H), 7.10 (d, J=8.6 Hz, 1H), 7.01 (d, J=7.0 Hz, 1H), 6.79 (d, J=5.9 Hz, 1H), 6.62 (t, J=5.9 Hz, 1H), 5.05 (m, 1H), 3.71 (q, J=7.3 Hz, 2H), 3.51 (m, 6H), 3.34 (m, 2H), 3.29 (m, 2H), 3.19 (t, J=7.3 Hz, 2H), 3.02 (q, J=6.0 Hz, 2H), 2.87 (m, 1H), 2.56 (m, 2H), 2.30 (m, 2H), 2.14 (t. J=7.4 Hz, 2H), 2.02 (m, 1H), 1.77 (m, 2H), 1.47 (m, 2H), 1.36 (m, 2H), 1.22 (s, 12H).

N-[12-[4-[6-[2-[(6-cyano-4-quinolyl)amino]ethyl]naphthalene-2-carbonyl]piperazin-1-yl]-12-oxo-dodecyl]-3-[[2-(2,6-dioxo-3-piperidyl)-1,3-dioxo-isoindolin-4-yl]amino]propanamide (49e (m=11, n=2)): A yellow solid (34 mg, 72% yield). ESI-MS m/z: 961 ([M+H]⁺). ¹H-NMR (300 MHz, DMSO-d6): 11.10 (s, 1H), 8.87 (s, 1H), 8.52 (d, J=5.9 Hz, 1H), 8.10 (m, 1H), 7.97 (s, 1H), 7.94 (d, J=4.2 Hz, 2H), 7.89 (d, J=4.2 Hz, 2H), 7.88 (s, 2H), 7.73 (t, J=5.4 Hz, 1H), 7.59 (d, J=7.5 Hz, 2H), 7.52 (t, J=7.0 Hz, 1H), 7.12 (d, J=8.6 Hz, 1H), 7.02 (d, J=7.0 Hz, 1H), 6.72 (d, J=5.9 Hz, 1H), 6.69 (t, J=5.9 Hz, 1H), 5.04 (m, 1H), 3.66 (q, J=7.3 Hz, 2H), 3.50 (m, 10H), 3.18 (t, J=7.3 Hz, 2H), 3.02 (q, J=6.0 Hz, 2H), 2.88 (m, 1H), 2.55 (m, 2H), 2.39 (t, J=6.2 Hz, 2H), 2.31 (m, 2H), 2.00 (m, 1H), 1.47 (m, 2H), 1.34 (m, 2H), 1.19 (s, 14H).

N-[10-[4-[6-[2-[(6-cyano-4-quinolyl)amino]ethyl]naphthalene-2-carbonyl]piperazin-1-yl]-10-oxo-decyl]-5-[[2-(2,6-dioxo-3-piperidyl)-1,3-dioxo-isoindolin-4-yl]amino]pentanamide (49f (m=9, n=4)): A yellow solid (28 mg, 22% yield) ESI-MS m/z: 961 ([M+H]⁺). ¹H-NMR (300 MHz, CD₂Cl₂): 8.95 (m, 1H), 8.59 (d, J=5.7 Hz, 1H), 8.21 (s, 1H), 8.00 (d, J=8.9 Hz, 1H), 7.88 (s, 1H), 7.83 (t, J=8.3 Hz, 2H), 7.71 (d, J=8.9 Hz, 1H), 7.69 (s, 1H), 7.48 (d, J=8.4 Hz, 2H), 7.45 (t, J=8.3 Hz, 2H), 7.02 (d, J=7.2 Hz, 1H), 6.87 (d, J=8.2 Hz, 1H), 6.57 (d, J=5.7 Hz, 1H), 6.24 (t, J=5.5 Hz, 1H), 5.93 (m, 1H), 5.74 (t, J=5.5 Hz, 1H), 4.90 (m, 1H), 3.68 (q. J=5.5 Hz, 2H), 3.54 (m, 6H), 3.21 (m, 6H), 2.78 (m, 2H), 2.71 (m, 1H), 2.31 (m, 2H), 2.17 (t, J=6.5 Hz, 2H), 2.10 (m, 1H), 1.69 (m, 4H), 1.58 (m, 2H), 1.44 (m, 2H), 1.26 (s, 12H).

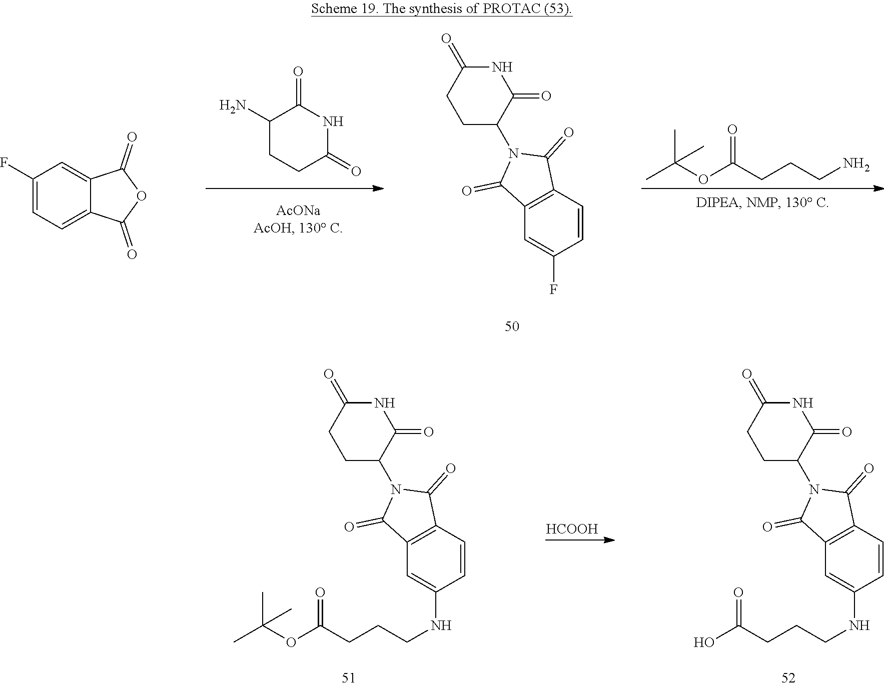

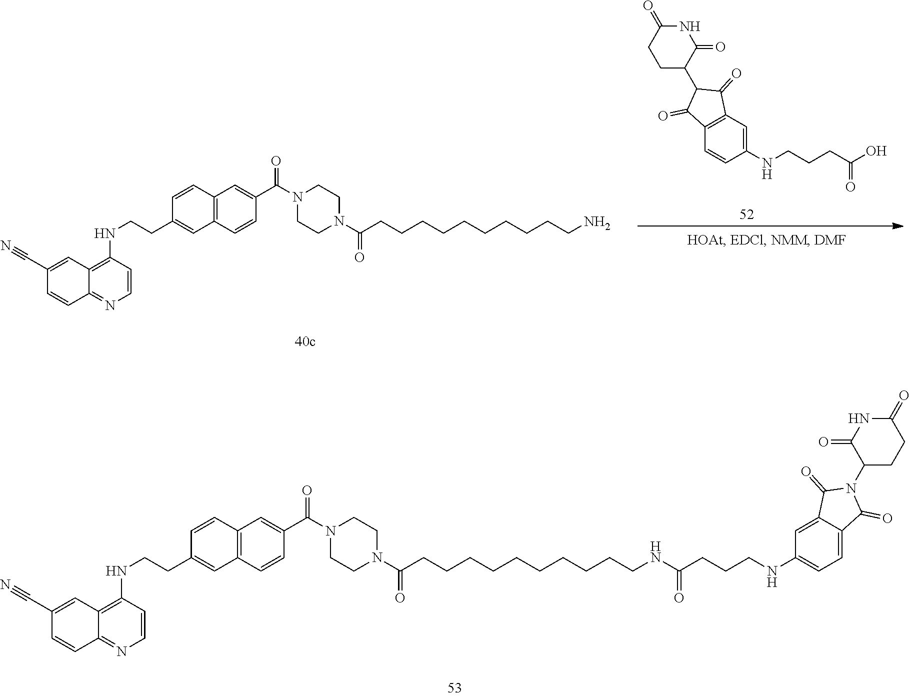 [SMILES: N#Cc1ccc2nccc(NCCc3ccc4cc(C(=O)N5CCN(C(=O)CCCCCCCCCCN)CC5)ccc4c3)c2c1 ; O=C(O)CCCNc1ccc2c(c1)C(=O)C(C1CCC(=O)NC1=O)C2=O ; N#Cc1ccc2nccc(NCCc3ccc4cc(C(=O)N5CCN(C(=O)CCCCCCCCCCNC(=O)CCCNc6ccc7c(c6)C(=O)N(C6CCC(=O)NC6=O)C7=O)CC5)ccc4c3)c2c1]

2-(2,6-dioxo-3-piperidyl)-5-fluoro-isoindoline-1,3-dione (50): To a 100 mL round-bottle flask was added a solution of 5-fluoroisobenzofuran-1,3-dione (6.02 mmol, 1 g) and 3-aminopiperidine-2,6-dione (6.02 mmol, 771 mg) in acetic acid (10 mL). Then sodium acetate (12 mmol, 988 mg) was added and the mixture was stirred at 130° C. for overnight. Upon completion, the mixture was cooled to room temperature, condensed. The residue was added with ice water, the precipitation was filtered and washed with water and EtOH, dried to give 2-(2,6-dioxo-3-piperidyl)-5-fluoro-isoindoline-1,3-dione (1.35 g, 81% yield) and used for the next step without further purification. ESI-MS m/z: 277 ([M+H]⁺).

tert-butyl 4-((2-(2,6-dioxopiperidin-3-yl)-1,3-dioxoisoindolin-S-yl)amino) butanoate (51): To a 20 mL microwave flask was added a solution of 2-(2,6-dioxo-3-piperidyl)-5-fluoro-isoindoline-1,3-dione (1.67 mmol, 266 mg), tert-butyl 4-aminobutanoate (1.67 mmol, 266 mg), and DIEA (4.55 mmol, 588 mg) in NMP (5 mL). Then the mixture was microwaved at 130° C. for 1 h. Upon completion, the mixture was poured in EtOAc, and washed with water and brine. The organic phase was dried by Na₂SO₄, condensed to give tert-butyl 4-((2-(2,6-dioxopiperidin-3-yl)-1,3-dioxoisoindolin-5-yl)amino) butanoate and used for the next step without further purification. ESI-MS m/z: 416 ([M+H]⁺).

4-[[2-(2,6-dioxo-3-piperidyl)-1,3-dioxo-isoindolin-5-yl]amino]butanoic acid (52): To a 10 mL round bottle flask was added a solution of tert-butyl 4-((2-(2,6-dioxopiperidin-3-yl)-1,3-dioxoisoindolin-5-yl)amino) butanoate in formic acid. The mixture was then stirred at room temperature for overnight. Upon completion, the mixture was condensed to give 4-[[2-(2,6-dioxo-3-piperidyl)-1,3-dioxo-isoindolin-5-yl]amino]butanoic acid (170 mg, 31% yield) and used for the next step without further purification. ESI-MS m/z: 360 ([M+H]⁺).

N-[11-[4-[6-[2-[(6-cyano-4-quinolyl)amino]ethyl]naphthalene-2-carbonyl]piperazin-1-yl]-11-oxo-undecyl]-4-[[2-(2,6-dioxo-3-piperidyl)-1,3-dioxo-isoindolin-5-yl]amino]butanamide (53): To a 10 mL round-bottle flask was added a solution of 4-[2-[6-[4-(11-aminoundecanoyl) piperazine-1-carbonyl]-2-naphthyl]ethylamino]quinoline-6-carbonitrile (0.08 mmol, 50 mg) and 4-[[2-(2,6-dioxo-3-piperidyl)-1,3-dioxo-isoindolin-5-yl]amino]butanoic acid (0.08 mmol, 29 mg) in DMF (2 mL). Then EDC-HCl (0.16 mmol, 31 mg), HOAt (0.16 mmol, 22 mg), and N-methylmorpholine (0.40 mmol, 41 mg) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give N-[11-[4-[6-[2-[(6-cyano-4-quinolyl)amino]ethyl]naphthalene-2-carbonyl]piperazin-1-yl]-11-oxo-undecyl]-4-[[2-(2,6-dioxo-3-piperidyl)-1,3-dioxo-isoindolin-5-yl]amino]butanamide (46 mg, 59% yield). ESI-MS m/z: 961 ([M+H]⁺). ¹H-NMR (300 MHz, DMSO-d6): 11.10 (s, 1H), 8.93 (s, 1H), 8.53 (d, J=5.9 Hz, 1H), 8.10 (m, 1H), 7.97 (s, 2H), 7.94 (s, 1H), 7.92 (s, 1H), 7.89 (s, 2H), 7.81 (t, J=5.4 Hz, 1H), 7.58 (d, J=7.5 Hz, 2H), 7.52 (t, J=7.0 Hz, 1H), 7.10 (d, J=8.6 Hz, 1H), 7.01 (d, J=7.0 Hz, 1H), 6.79 (d, J=5.9 Hz, 1H), 6.62 (t, J=5.9 Hz, 1H), 5.05 (m, 1H), 3.71 (q, J=7.3 Hz, 2H), 3.51 (m, 6H), 3.34 (m, 2H), 3.29 (m, 2H), 3.19 (t, J=7.3 Hz, 2H), 3.02 (q, J=6.0 Hz, 2H), 2.87 (m, 1H), 2.56 (m, 2H), 2.30 (m, 2H), 2.14 (t, J=7.4 Hz, 2H), 2.02 (m, 1H), 1.77 (m, 2H), 1.47 (m, 2H), 1.36 (m, 2H), 1.22 (s, 12H).

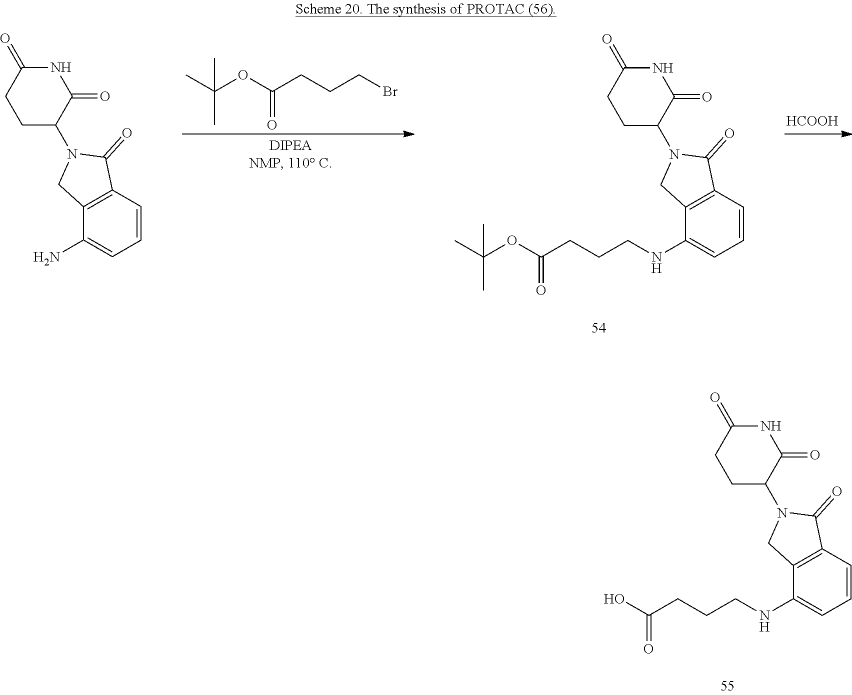 [SMILES: Nc1cccc2c1CN(C1CCC(=O)NC1=O)C2=O ; CC(C)(C)OC(=O)CCCBr ; CC(C)(C)OC(=O)CCCNc1cccc2c1CN(C1CCC(=O)NC1=O)C2=O ; "HCOOH" ; O=C(O)CCCNc1cccc2c1CN(C1CCC(=O)NC1=O)C2=O]

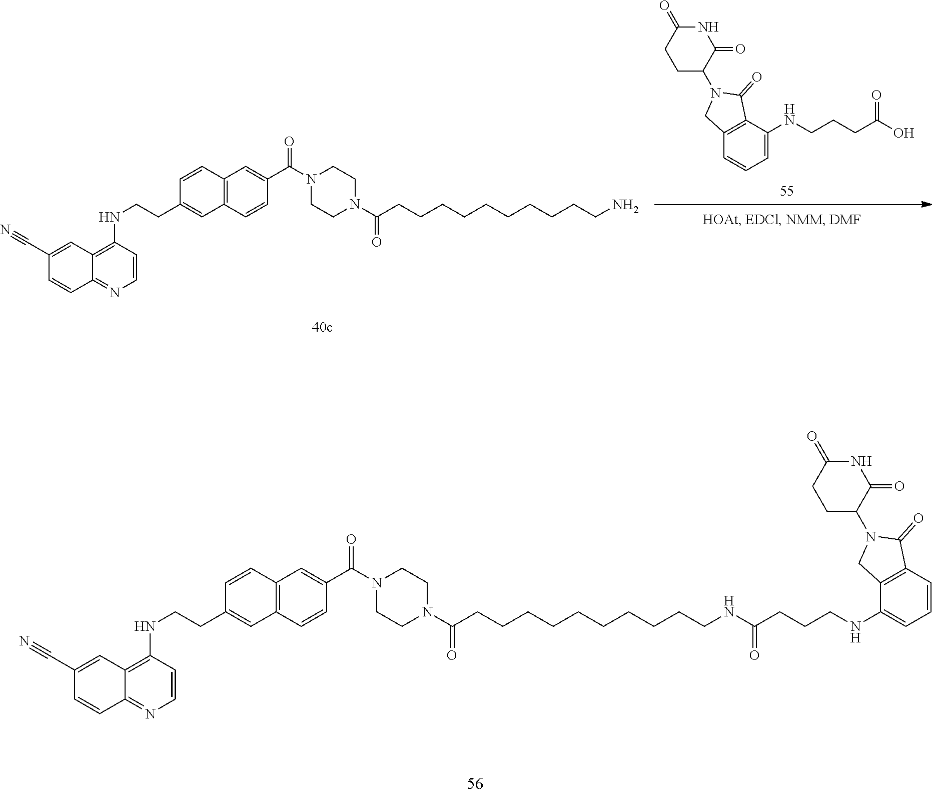 [SMILES: N#Cc1ccc2nccc(NCCc3ccc4cc(C(=O)N5CCN(C(=O)CCCCCCCCCCN)CC5)ccc4c3)c2c1 ; O=C(O)CCCNc1cccc2c1C(=O)N(C1CCC(=O)NC1=O)C2 ; N#Cc1ccc2nccc(NCCc3ccc4cc(C(=O)N5CCN(C(=O)CCCCCCCCCCNC(=O)CCCNc6cccc7c6CN(C6CCC(=O)NC6=O)C7=O)CC5)ccc4c3)c2c1]

tert-butyl 4-[[2-(2,6-dioxo-3-piperidyl)-1-oxo-isoindolin-4-yl]amino]butanoate (54): To a 100 mL round-bottom flask was added a solution of 3-(4-amino-1-oxo-isoindolin-2-yl) piperidine-2,6-dione (3.86 mmol, 1 g), tert-butyl 4-bromobutanoate (4.24 mmol, 947 mg), and DIEA (11.6 mmol, 1.5 g) in NMP (5 mL). Then the mixture was stirred at 130° C. for overnight. Upon completion, the mixture was cooled to room temperature and poured into EtOAc, washed with water and brine. The organic phase was dried by Na₂SO₄, condensed and purified by flash column chromatography using a gradient of 0-5% MeOH/DCM to give tert-butyl 4-[[2-(2,6-dioxo-3-piperidyl)-1-oxo-isoindolin-4-yl]amino]butanoate (538 mg, 35% yield). ESI-MS m/z: 360 ([M+H]⁺). ¹H-NMR (300 MHz, DMSO-d6): 11.02 (s, 1H), 7.28 (t, J=7.7 Hz, 1H), 6.93 (d, J=7.5 Hz, 1H), 6.75 (d, J=7.7 Hz, 1H), 5.65 (t, J=5.7 Hz, 1H), 5.12 (dd, J=13.8, 5.2 Hz, 1H), 4.17 (q, J=17.2 Hz, 2H), 3.13 (q, J=7.3 Hz, 2H), 2.93 (m, 1H), 2.62 (m, 1H), 2.33 (t, J=7.9 Hz, 2H), 2.28 (m, 1H), 2.03 (m, 1H), 1.79 (m, 2H), 1.39 (s, 9H).

tert-butyl 4-[[2-(2,6-dioxo-3-piperidyl)-1-oxo-isoindolin-4-yl]amino]butanoate (55): To a 10 mL round-bottom flask was added a solution of tert-butyl 4-[[2-(2,6-dioxo-3-piperidyl)-1-oxo-isoindolin-4-yl]amino]butanoate (0.4 mmol, 160 mg) in formic acid (3 mL). Then the mixture was stirred at room temperature for overnight. Upon completion, the mixture was condensed and purified by flash column chromatography using a gradient of 0-7% MeOH/DCM to give 4-[[2-(2,6-dioxo-3-piperidyl)-1-oxo-isoindolin-4-yl]amino]butanoic acid (126 mg, 92% yield). ESI-MS m/z: 346 ([M+H]⁺). ¹H-NMR (300 MHz, DMSO-d6): 12.23 (s, 1H), 11.02 (s, 1H), 7.28 (t, J=7.7 Hz, 1H), 6.93 (d, J=7.5 Hz, 1H), 6.76 (d, J=7.7 Hz, 1H), 5.65 (m, 1H), 5.12 (dd, J=12.8, 5.2 Hz, 1H), 4.18 (q, J=17.2 Hz, 2H), 3.14 (m, 2H), 2.93 (m, 1H), 2.62 (m, 1H), 2.34 (t, J=7.9 Hz, 2H), 2.24 (m, 1H), 2.04 (m, 1H), 1.80 (m, 2H).

N-[11-[4-[6-[2-[(6-cyano-4-quinolyl)amino]ethyl]naphthalene-2-carbonyl]piperazin-1-yl]-11-oxo-undecyl]-4-[[2-(2,6-dioxo-3-piperidyl)-1-oxo-isoindolin-4-yl]amino]butanamide (56): To a 10 mL round-bottle flask was added a solution of 4-[2-[6-[4-(11-aminoundecanoyl) piperazine-1-carbonyl]-2-naphthyl]ethylamino]quinoline-6-carbonitrile (0.08 mmol, 50 mg) and 4-[[2-(2,6-dioxo-3-piperidyl)-1-oxo-isoindolin-4-yl]amino]butanoic acid (0.08 mmol, 28 mg) in DMF (2 mL). Then EDC-HCl (0.16 mmol, 31 mg), HOAt (0.16 mmol, 22 mg), and N-methylmorpholine (0.40 mmol, 41 mg) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give N-[11-[4-[6-[2-[(6-cyano-4-quinolyl)amino]ethyl]naphthalene-2-carbonyl]piperazin-1-yl]-11-oxo-undecyl]-4-[[2-(2,6-dioxo-3-piperidyl)-1-oxo-isoindolin-4-yl]amino]butanamide (36 mg, 47% yield). ESI-MS m/z: 947 ([M+H]⁺). ¹H-NMR (300 MHz, DMSO-d6): 11.02 (s, 1H), 8.86 (s, 1H), 8.52 (d, J=5.9 Hz, 1H), 7.97 (s, 1H), 7.93 (d, J=4.9 Hz, 1H), 7.89 (s, 1H), 7.88 (s, 3H), 7.78 (t, J=5.4 Hz, 1H), 7.71 (t, J=5.4 Hz, 1H), 7.58 (d, J=7.8 Hz, 1H), 7.50 (d, J=7.8 Hz, 1H), 7.27 (t, J=7.8 Hz, 1H), 6.92 (d, J=7.4 Hz, 1H), 6.73 (d, J=7.8 Hz, 1H), 6.71 (d, J=5.4 Hz, 1H), 5.64 (m, 1H), 5.11 (m, 1H), 4.16 (q, J=17.0 Hz, 2H), 3.65 (q, J=6.9 Hz, 2H), 3.51 (m, 6H), 3.19 (t, J=7.6 Hz, 2H), 3.00 (m, 4H), 2.61 (m, 1H), 2.30 (m, 3H), 2.17 (t, J=7.6 Hz, 1H), 2.03 (m, 2H), 1.79 (m, 1H), 1.47 (m, 2H), 1.34 (m, 2H), 1.22 (s, 12H).

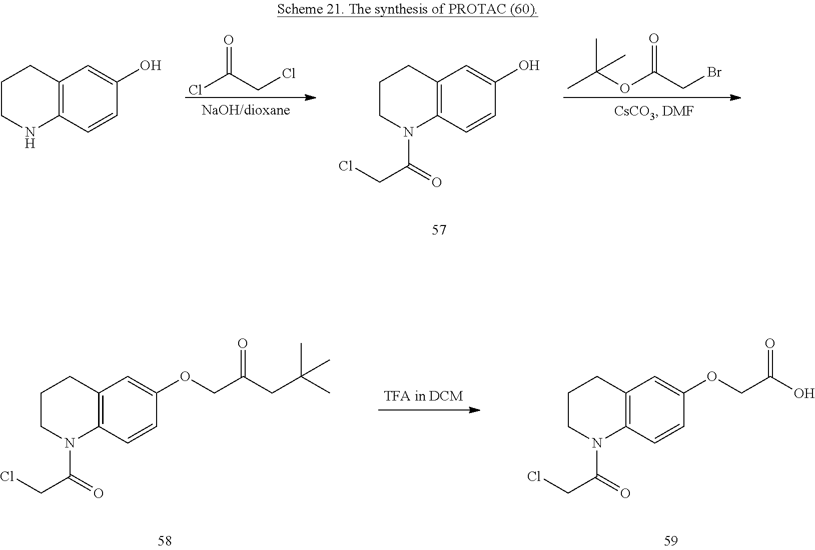

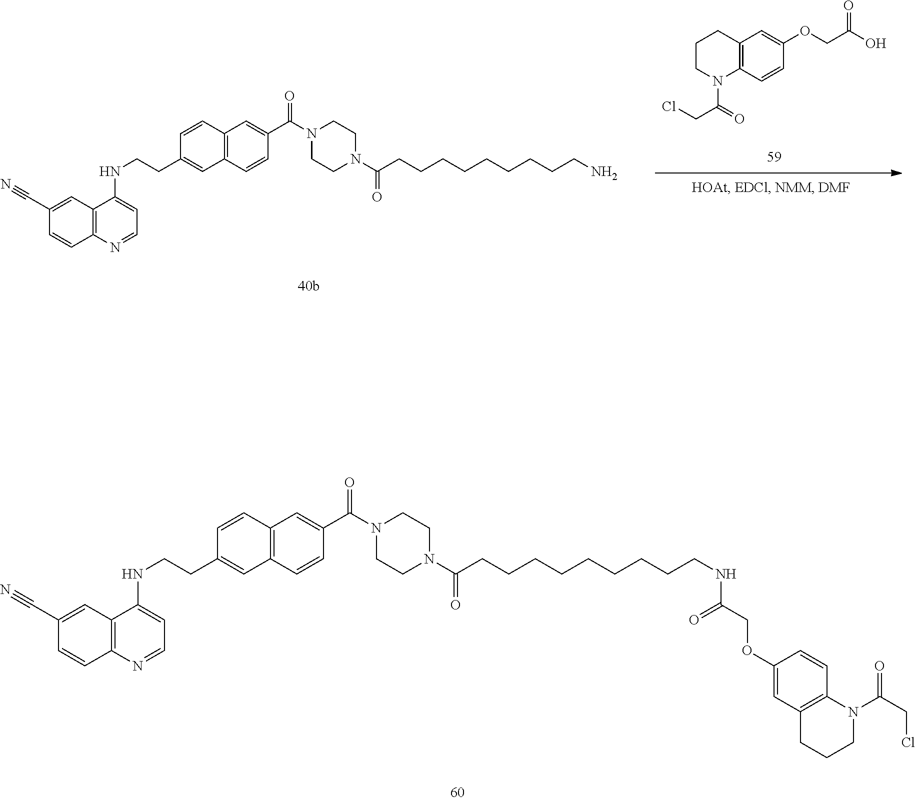 [SMILES: N#Cc1ccc2nccc(NCCc3ccc4cc(C(=O)N5CCN(C(=O)CCCCCCCCCN)CC5)ccc4c3)c2c1 ; O=C(O)COc1ccc2c(c1)CCCN2C(=O)CCl ; N#Cc1ccc2nccc(NCCc3ccc4cc(C(=O)N5CCN(C(=O)CCCCCCCCCNC(=O)COc6ccc7c(c6)CCCN7C(=O)CCl)CC5)ccc4c3)c2c1]

2-chloro-1-(6-hydroxy-3,4-dihydro-2H-quinolin-1-yl)ethanone (57): To a 100 mL round-bottle flask was added a solution of 1,2,3,4-tetrahydroquinolin-6-ol (6.70 mmol, 1 g) and NaOH (8.0 mmol, 320 mg) in water/dioxane (1:1, 20 mL) at 0° C. Then chloroacetyl chloride (7.40 mmol, 0.59 mL) was added dropwise over 5 minutes and then the reaction was stirred at room temperature for 4 h. Upon completion, the reaction mixture was acidified with 1n HCl to pH 4, extracted with ethyl acetate. The organic layers were combined and washed with brine, dried over anhydrous Na₂SO₄, condensed and purified by flash column chromatography using a gradient of 0-4% MeOH/DCM to give 2-chloro-1-(6-hydroxy-3,4-dihydro-2H-quinolin-1-yl)ethanone (1.4 g, 93% yield). ESI-MS m/z: 226 ([M+H]⁺). ¹H-NMR (300 MHz, DMSO-d6): 9.43 (s, 1H), 7.33 (m, 1H), 6.59 (s, 2H), 4.38 (s, 2H), 3.64 (t, J=5.9 Hz, 2H), 2.61 (m, 2H), 1.84 (m, 2H).

tert-butyl 2-[[1-(2-chloroacetyl)-3,4-dihydro-2H-quinolin-6-yl]oxy]acetate (58): To a 100 mL round-bottle flask was added a solution of 2-chloro-1-(6-hydroxy-3,4-dihydro-2H-quinolin-1-yl)ethanone (2.22 mmol, 500 mg) in DMF (5 mL). Then Cs₂CO₃ (3.32 mmol, 1.08 g) and tert-butyl 2-bromoacetate (2.77 mmol, 540 mg) were added and the reaction was stirred at room temperature for 3 h. Upon completion, the reaction mixture was diluted with ethyl acetate, acidified with 1n HCl to pH 4, extracted with ethyl acetate. The organic layers were combined and washed with brine, dried over anhydrous Na₂SO₄, condensed and to give tert-butyl 2-[[1-(2-chloroacetyl)-3,4-dihydro-2H-quinolin-6-yl]oxy]acetate (550 mg, 73% yield) and used without further purification. ESI-MS m/z: 340 ([M+H]⁺). ¹H-NMR (300 MHz, DMSO-d6): 7.47 (m, 1H), 6.74 (m, 2H), 4.62 (s, 2H), 4.47 (s, 2H), 3.66 (t, J=5.9 Hz, 2H), 2.67 (m, 2H), 1.87 (m, 2H), 1.43 (s, 9H).

4-[[1-(2-chloroacetyl)-3,4-dihydro-2H-quinolin-6-yl]oxy]butanoic acid (59): To a 10 mL round-bottle flask was added a solution of tert-butyl 2-[[1-(2-chloroacetyl)-3,4-dihydro-2H-quinolin-6-yl]oxy]acetate (0.88 mmol, 300 mg) in DCM (5 mL). Then TFA (1 mL) was added and the reaction was stirred at room temperature for 4 h. Upon completion, the reaction mixture was condensed and to give 2-[[1-(2-chloroacetyl)-3,4-dihydro-2H-quinolin-6-yl]oxy]acetic acid (220 mg, 88% yield) and used without further purification. ESI-MS m/z: 284 ([M+H]⁺). ¹H-NMR (300 MHz, DMSO-d6): 12.99 (s, 1H), 7.47 (m, 1H), 6.75 (m, 2H), 4.65 (s, 2H), 4.47 (m, 2H), 3.66 (t, J=5.9 Hz, 2H), 3.40 (m, 2H), 2.68 (m, 2H), 1.87 (m, 2H).

2-[[1-(2-chloroacetyl)-3,4-dihydro-2H-quinolin-6-yl]oxy]-N-[10-[4-[6-[2-[(6-cyano-4-quinolyl)amino]ethyl]naphthalene-2-carbonyl]piperazin-1-yl]-10-oxo-decyl]acetamide (60): To a 10 mL round-bottle flask was added a solution of 4-[2-[6-[4-(10-aminodecanoyl) piperazine-1-carbonyl]-2-naphthyl]ethylamino]quinoline-6-carbonitrile (0.057 mmol, 34 mg) and 2-[[1-(2-chloroacetyl)-3,4-dihydro-2H-quinolin-6-yl]oxy]acetic acid (0.057 mmol, 16 mg) in DMF (2 mL). Then EDC-HCl (0.11 mmol, 22 mg), HOAt (0.11 mmol, 15 mg), and N-methylmorpholine (0.28 mmol, 29 mg) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give 2-[[1-(2-chloroacetyl)-3,4-dihydro-2H-quinolin-6-yl]oxy]-N-[10-[4-[6-[2-[(6-cyano-4-quinolyl)amino]ethyl]naphthalene-2-carbonyl]piperazin-1-yl]-10-oxo-decyl]acetamide (15 mg, 31% yield). ESI-MS m/z: 871 ([M+H]⁺). ¹H-NMR (300 MHz, CD₂Cl₂): 8.58 (d, J=4.9 Hz, 1H), 8.27 (s, 1H), 7.96 (d, J=8.2 Hz, 1H), 7.88 (s, 1H), 7.83 (d, J=8.9 Hz, 2H), 7.72 (d, J=9.7 Hz, 1H), 7.69 (s, 1H), 7.48 (d, J=8.2 Hz, 1H), 7.43 (d, J=8.2 Hz, 1H), 7.11 (m, 1H), 6.77 (m, 2H), 6.59 (d, J=5.8 Hz, 2H), 5.97 (m, 1H), 4.43 (s, 2H), 4.20 (m, 2H), 3.74 (m, 2H), 3.69 (q, J=5.6 Hz, 2H), 3.58 (m, 6H), 3.29 (q, J=6.6 Hz, 2H), 3.20 (t, J=6.6 Hz, 2H), 2.70 (m, 2H), 2.31 (m, 2H), 1.95 (m, 2H), 1.58 (m, 2H), 1.51 (m, 2H), 1.28 (s, 12H).

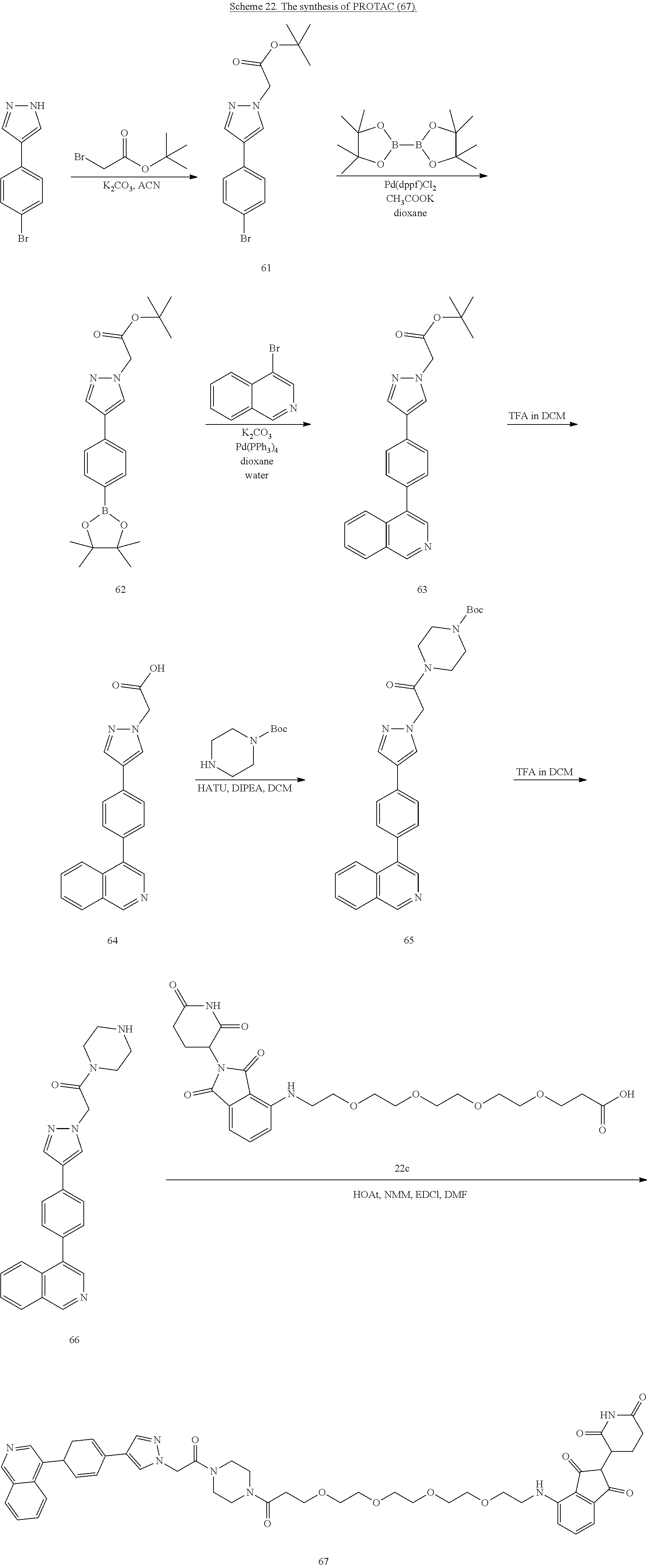

tert-butyl 2-[4-(4-bromophenyl) pyrazol-1-yl]acetate (61): To a 100 mL round-bottle flask was added a solution of 4-(4-bromophenyl)-1H-pyrazole (5.38 mmol, 1.00 g) and tert-butyl 2-bromoacetate (5.38 mmol, 1.05 g) in acetonitrile (10 mL). Then K₂CO₃ (4.30 mmol, 1.45 g) was added and the mixture was stirred at room temperature for overnight. Upon completion, the mixture was condensed, and sat NH₄Cl aq was added, the mixture was extracted with DCM, the organic layers were combined, dried by Na₂SO₄, condensed and purified by flash column chromatography using a gradient of 0-2% MeOH/DCM to give tert-butyl 2-[4-(4-bromophenyl) pyrazol-1-yl]acetate (1.45 g, 96% yield). ESI-MS m/z: 338 ([M+H]⁺). ¹H-NMR (300 MHz, DMSO-d6): 8.20 (s, 1H), 7.95 (s, 1H), 7.54 (s, 4H), 4.96 (s, 2H), 1.43 (s, 9H).

tert-butyl 2-[4-[4-(4,4,5,5-tetramethyl-1,3,2-dioxaborolan-2-yl)phenyl]pyrazol-1-yl]acetate (62): To a 100 mL round-bottle flask was added a solution of tert-butyl 2-[4-(4-bromophenyl) pyrazol-1-yl]acetate (1.48 mmol, 500 mg) and 4,4,5,5-tetramethyl-2-(4,4,5,5-tetramethyl-1,3,2-dioxaborolan-2-yl)-1,3,2-dioxaborolane (2.22 mmol, 565 mg) in dioxane (10 mL). Then CH₃COOK (4.45 mmol, 437 mg) and Pd(dppf)Cl₂ (0.148 mmol, 108 mg) were added and the mixture was degassed and stirred at 95° C. for overnight. Upon completion, the mixture was cooled to room temperature, then sat NaHCO₃ aq was added, the mixture was extracted with DCM, the organic layers were combined, dried by Na₂SO₄, condensed and purified by flash column chromatography using a gradient of 0-2% MeOH/DCM to give tert-butyl 2-[4-[4-(4,4,5,5-tetramethyl-1,3,2-dioxaborolan-2-yl)phenyl]pyrazol-1-yl]acetate (550 mg, 97% yield). ESI-MS m/z: 385 ([M+H]⁺). ¹H-NMR (300 MHz, DMSO-d6): 8.21 (s, 1H), 7.96 (s, 1H), 7.63 (q, J=8.1 Hz, 4H), 4.96 (s, 2H), 1.43 (s, 9H), 1.29 (s, 12H).

tert-butyl 2-(4-(4-(isoquinolin-4-yl)phenyl)-1H-pyrazol-1-yl) acetate (63): To a 100 mL round-bottle flask was added a solution of tert-butyl 2-[4-[4-(4,4,5,5-tetramethyl-1,3,2-dioxaborolan-2-yl)phenyl]pyrazol-1-yl]acetate (1.41 mmol, 540 mg) and 4-bromoisoquinoline (1.41 mmol, 292 mg) in dioxane (10 mL). Then K₂CO₃ (4.22 mmol, 583 mg) and Tetrakis (triphenylphosphine) palladium (O) (0.14 mmol, 162 mg) were added and the mixture was degassed and stirred at 100° C. for overnight. Upon completion, the mixture was cooled to room temperature, then sat NaHCO₃ aq was added, the mixture was extracted with DCM, the organic layers were combined, dried by Na₂SO₄, condensed to give tert-butyl 2-(4-(4-(isoquinolin-4-yl)phenyl)-1H-pyrazol-1-yl) acetate (500 mg, 92%) and used for the next step without further purification. ESI-MS m/z: 386 ([M+H]⁺).

2-(4-(4-(isoquinolin-4-yl)phenyl)-1H-pyrazol-1-yl) acetic acid (64): To a 10 mL round-bottle flash was added a solution of tert-butyl 2-(4-(4-(isoquinolin-4-yl)phenyl)-1H-pyrazol-1-yl) acetate (500 mg) dissolved in DCM (5 mL). Then TFA (2 mL) was added and the mixture was stirred at room temperature for 4 h. Upon completion, the mixture was condensed to give 2-(4-(4-(isoquinolin-4-yl)phenyl)-1H-pyrazol-1-yl) acetic acid (400 mg, 94%) and used for the next step without further purification. ESI-MS m/z: 330 ([M+H]⁺).

tert-butyl 2-[4-[4-(4,4,5,5-tetramethyl-1,3,2-dioxaborolan-2-yl)phenyl]pyrazol-1-yl]acetate (65): To a 10 mL round-bottle flash was added a solution of 2-(4-(4-(isoquinolin-4-yl)phenyl)-1H-pyrazol-1-yl) acetic acid (400 mg, 1.21 mmol) HATU (1.82 mmol, 691 mg) and DIEA (3.64 mmol, 624 μL) were added and the mixture was stirred at room temperature for 15 min, then tert-butyl piperazine-1-carboxylate (1.21 mmol, 226 mg) was added and the mixture was stirred at room temperature for 4 h. Upon completion, sat NaHCO₃ aq was added and the mixture was extracted with DCM, the organic layers were combined and dried over Na₂SO₄, condensed and purified by flash column chromatography using a gradient of 0-5% MeOH/DCM to give tert-butyl 4-[2-[4-[4-(4-isoquinolyl)phenyl]pyrazol-1-yl]acetyl]piperazine-1-carboxylate (247 mg, 35% yield). ESI-MS m/z: 498 ([M+H]⁺). ¹H-NMR (300 MHz, DMSO-d6): 9.24 (s, 1H), 8.48 (s, 1H), 8.06 (d, J=7.9 Hz, 1H), 7.97 (d, J=7.9 Hz, 1H), 7.88 (s, 2H), 7.68 (m, 4H), 7.54 (d, J=7.9 Hz, 2H), 5.06 (s, 2H), 3.59 (m, 2H), 3.52 (m, 2H), 3.44 (m, 4H), 1.45 (s, 9H).

2-[4-[4-(4-isoquinolyl)phenyl]pyrazol-1-yl]-1-piperazin-1-yl-ethanone (66): To a 10 mL round-bottle flask was added a solution of tert-butyl 4-[2-[4-[4-(4-isoquinolyl)phenyl]pyrazol-1-yl]acetyl]piperazine-1-carboxylate (0.47 mmol, 233 mg) in DCM (5 mL). Then TFA (1 mL) was added and the reaction was stirred at room temperature for 4 h. Upon completion, the reaction mixture was condensed and to give 2-[4-[4-(4-isoquinolyl)phenyl]pyrazol-1-yl]-1-piperazin-1-yl-ethanone (186 mg, 99% yield) and used without further purification. ESI-MS m/z: 398 ([M+H]⁺).

2-(2,6-dioxo-3-piperidyl)-4-[2-[2-[2-[2-[3-[4-[2-[4-[4-(4-isoquinolyl)phenyl]pyrazol-1-yl]acetyl]piperazin-1-yl]-3-oxo-propoxy]ethoxy]ethoxy]ethoxy]ethylamino]isoindoline-1,3-dione (67): To a 10 mL round-bottle flask was added a solution of 2-[4-[4-(4-isoquinolyl)phenyl]pyrazol-1-yl]-1-piperazin-1-yl-ethanone (0.091 mmol, 36 mg) and 3-[2-[2-[2-[2-[[2-(2,6-dioxo-3-piperidyl)-1,3-dioxo-isoindolin-4-yl]amino]ethoxy]ethoxy]ethoxy]ethoxy]propanoic acid (0.091 mmol, 47 mg) in DMF (2 mL). Then EDC-HCl (0.18 mmol, 35 mg), HOAt (0.18 mmol, 25 mg), and N-methylmorpholine (0.45 mmol, 46 mg) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give 2-(2,6-dioxo-3-piperidyl)-4-[2-[2-[2-[2-[3-[4-[2-[4-[4-(4-isoquinolyl)phenyl]pyrazol-1-yl]acetyl]piperazin-1-yl]-3-oxo-propoxy]ethoxy]ethoxy]ethoxy]ethylamino]isoindoline-1,3-dione (15 mg, 18% yield). ESI-MS m/z: 901 ([M+H]⁺). ¹H-NMR (300 MHz, CD₂Cl₂): 9.24 (s, 1H), 8.48 (s, 1H), 8.06 (d, J=7.9 Hz, 1H), 7.97 (d, J=7.9 Hz, 1H), 7.89 (s, 2H), 7.67 (m, 5H), 7.51 (m, 3H), 7.06 (d, J=7.5 Hz, 1H), 6.95 (d, J=8.2 Hz, 1H), 6.50 (t, J=5.3 Hz, 1H), 5.09 (s, 2H), 4.91 (m, 1H), 3.77-3.46 (m, 26H), 2.77 (m, 3H), 2.62 (m, 2H), 2.10 (m, 1H).

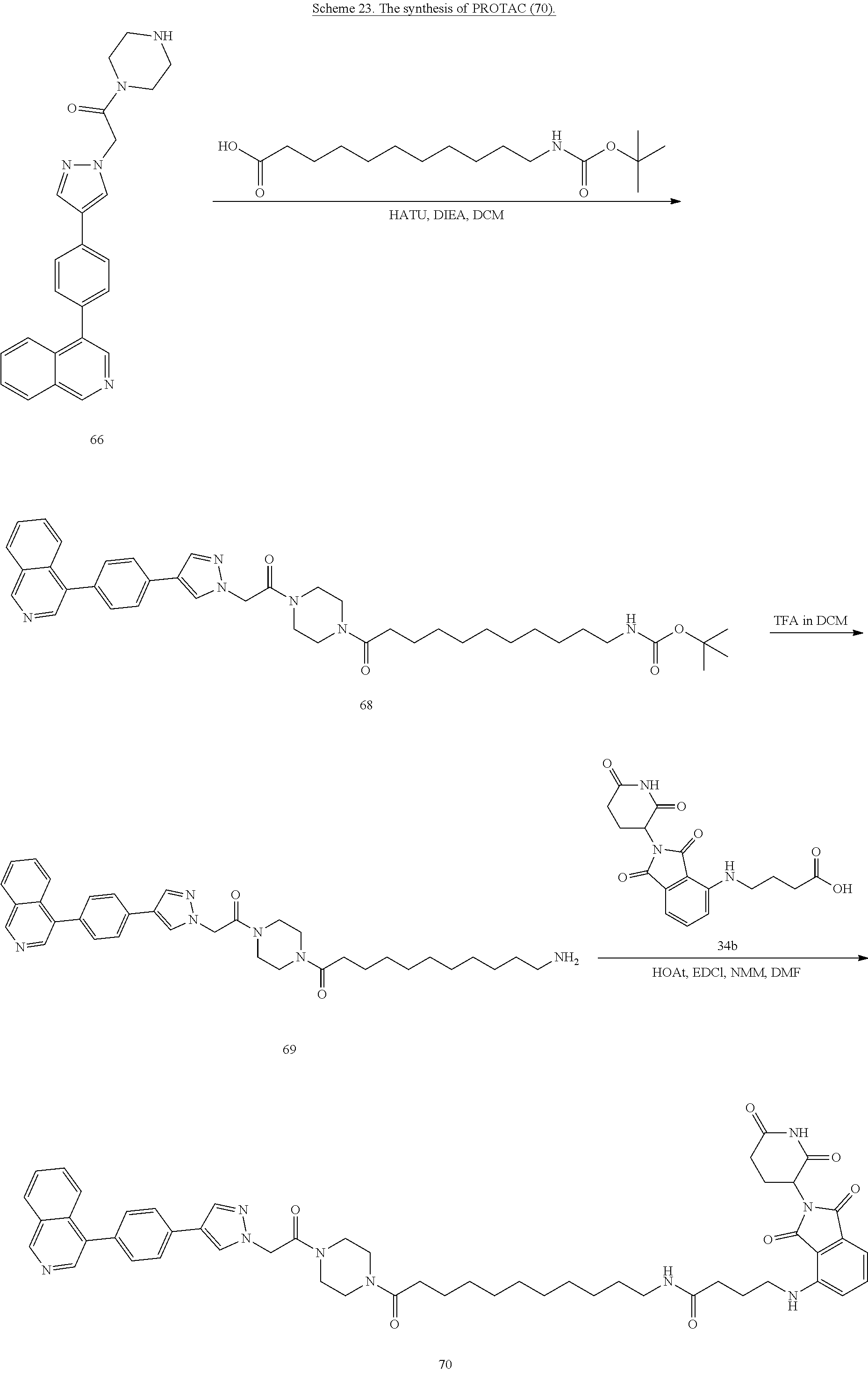 [SMILES: O=C(Cn1cc(-c2ccc(-c3cncc4ccccc34)cc2)cn1)N1CCNCC1 ; CC(C)(C)OC(=O)NCCCCCCCCCCC(=O)O ; CC(C)(C)OC(=O)NCCCCCCCCCCC(=O)N1CCN(C(=O)Cn2cc(-c3ccc(-c4cncc5ccccc45)cc3)cn2)CC1 ; NCCCCCCCCCCC(=O)N1CCN(C(=O)Cn2cc(-c3ccc(-c4cncc5ccccc45)cc3)cn2)CC1 ; O=C(O)CCCNc1cccc2c1C(=O)N(C1CCC(=O)NC1=O)C2=O ; O=C(CCCNc1cccc2c1C(=O)N(C1CCC(=O)NC1=O)C2=O)NCCCCCCCCCCC(=O)N1CCN(C(=O)Cn2cc(-c3ccc(-c4cncc5ccccc45)cc3)cn2)CC1]

tert-butyl N-[11-[4-[2-[4-[4-(4-isoquinolyl)phenyl]pyrazol-1-yl]acetyl]piperazin-1-yl]-11-oxo-undecyl]carbamate (68): To a 10 mL round-bottle flask was added a solution of 2-[4-[4-(4-isoquinolyl)phenyl]pyrazol-1-yl]-1-piperazin-1-yl-ethanone (0.81 mmol, 322 mg) in DCM (5 mL), HATU (1.22 mmol, 461 mg) and DIEA (2.43 mmol, 416 μL) were added and the mixture was stirred at room temperature for 15 min, then 11-(tert-butoxycarbonylamino) undecanoic acid (0.81 mmol, 244 mg) was added and the mixture was stirred at room temperature for 4 h. Upon completion, sat NaHCO₃ aq was added and the mixture was extracted with DCM, the organic layers were combined and dried over Na₂SO₄, condensed and purified by flash column chromatography using a gradient of 0-5% MeOH/DCM to give tert-butyl N-[11-[4-[2-[4-[4-(4-isoquinolyl)phenyl]pyrazol-1-yl]acetyl]piperazin-1-yl]-11-oxo-undecyl]carbamate (280 mg, 51% yield). ESI-MS m/z: 681 ([M+H]⁺). ¹H-NMR (300 MHz, CD₂Cl₂): 9.24 (s, 1H), 8.47 (s, 1H), 8.08 (d, J=7.3 Hz, 1H), 7.98 (d, J=7.3 Hz, 1H), 7.88 (s, 2H), 7.70 (s, 2H), 7.69 (m, 4H), 7.54 (d, J=8.3 Hz, 2H), 5.09 (s, 2H), 4.62 (m, 1H), 3.69-3.49 (m, 8H), 3.06 (q, J=6.4 Hz, 2H), 2.32 (t, J=7.7 Hz, m, 2H), 1.60 (m, 2H), 1.41 (m, 11H), 1.29 (s, 12H).

11-amino-1-[4-[2-[4-[4-(4-isoquinolyl) phenyl]pyrazol-1-yl]acetyl]piperazin-1-yl]undecan-1-one (69): To a 10 mL round-bottle flask was added a solution of tert-butyl tert-butyl N-[11-[4-[2-[4-[4-(4-isoquinolyl)phenyl]pyrazol-1-yl]acetyl]piperazin-1-yl]-11-oxo-undecyl]carbamate (0.41 mmol, 280 mg) in DCM (5 mL). Then TFA (1 mL) was added and the reaction was stirred at room temperature for 4 h. Upon completion, the reaction mixture was condensed and to give 11-amino-1-[4-[2-[4-[4-(4-isoquinolyl)phenyl]pyrazol-1-yl]acetyl]piperazin-1-yl]undecan-1-one (220 mg, 92% yield) and used without further purification. ESI-MS m/z: 581 ([M+H]⁺).

4-[[2-(2,6-dioxo-3-piperidyl)-1,3-dioxo-isoindolin-4-yl]amino]-N-[11-[4-[2-[4-[4-(4-isoquinolyl)phenyl]pyrazol-1-yl]acetyl]piperazin-1-yl]-11-oxo-undecyl]butanamide (70): To a 10 mL round-bottle flask was added a solution of 11-amino-1-[4-[2-[4-[4-(4-isoquinolyl)phenyl]pyrazol-1-yl]acetyl]piperazin-1-yl]undecan-1-one (0.11 mmol, 65 mg) and 4-[[2-(2,6-dioxo-3-piperidyl)-1,3-dioxo-isoindolin-4-yl]amino]butanoic acid (0.11 mmol, 40 mg) in DMF (2 mL). Then EDC-HCl (0.18 mmol, 35 mg), HOAt (0.22 mmol, 31 mg), and N-methylmorpholine (0.47 mmol, 103 mg) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give 4-[[2-(2,6-dioxo-3-piperidyl)-1,3-dioxo-isoindolin-4-yl]amino]-N-[11-[4-[2-[4-[4-(4-isoquinolyl)phenyl]pyrazol-1-yl]acetyl]piperazin-1-yl]-11-oxo-undecyl]butanamide (43 mg, 42% yield). ESI-MS m/z: 901 ([M+H]⁺). ¹H-NMR (300 MHz, CD₂Cl₂): 9.24 (s, 1H), 8.97 (m, 1H), 8.47 (s, 1H), 8.07 (d, J=7.9 Hz, 1H), 7.97 (d, J=7.9 Hz, 1H), 7.90 (s, 2H), 7.68 (m, 4H), 7.51 (m, 3H), 7.05 (d, J=7.3 Hz, 1H), 6.95 (d, J=8.7 Hz, 1H), 6.32 (t, J=6.0 Hz, 1H), 5.80 (m, 1H), 5.10 (s, 2H), 4.91 (m, 1H), 3.67-3.46 (m, 8H), 3.33 (q, J=7.2 Hz, 2H), 3.19 (q, J=6.5 Hz, 2H), 2.77 (m, 3H), 2.30 (m, 2H), 2.25 (t, J=7.9 Hz, 2H), 2.10 (m, 1H), 1.95 (m, 2H), 1.58 (m, 2H), 1.45 (m, 2H), 1.27 (s, 12H).

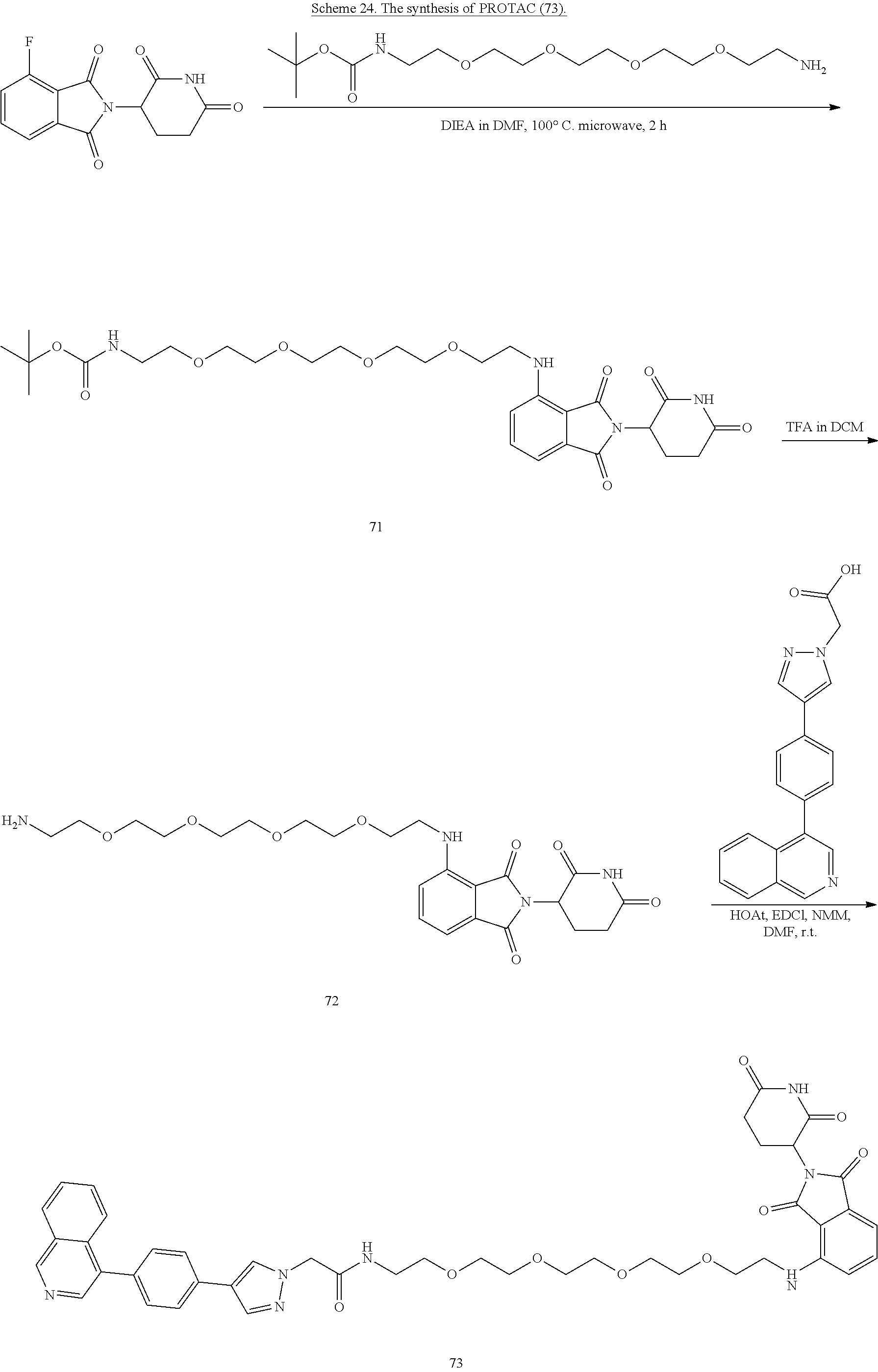 [SMILES: O=C1CCC(N2C(=O)c3cccc(F)c3C2=O)C(=O)N1 ; CC(C)(C)OC(=O)NCCOCCOCCOCCOCCN ; CC(C)(C)OC(=O)NCCOCCOCCOCCOCCNc1cccc2c1C(=O)N(C1CCC(=O)NC1=O)C2=O ; NCCOCCOCCOCCOCCNc1cccc2c1C(=O)N(C1CCC(=O)NC1=O)C2=O ; O=C(O)Cn1cc(-c2ccc(-c3cncc4ccccc34)cc2)cn1 ; O=C(Cn1cc(-c2ccc(-c3cncc4ccccc34)cc2)cn1)NCCOCCOCCOCCOCCNc1cccc2c1C(=O)N(C1CCC(=O)NC1=O)C2=O]

tert-butyl N-[2-[2-[2-[2-[2-[[2-(2,6-dioxo-3-piperidyl)-1,3-dioxo-isoindolin-4-yl]amino]ethoxy]ethoxy]ethoxy]ethoxy]ethyl]carbamate (71): To a 10 mL round-bottle flask was added a solution of 2-(2,6-dioxo-3-piperidyl)-4-fluoro-isoindoline-1,3-dione (0.72 mmol, 200 mg) and tert-butyl N-[2-[2-[2-[2-(2-aminoethoxy) ethoxy]ethoxy]ethoxy]ethyl]carbamate (0.87 mmol, 292 mg) in DMF (2 mL) Then DIEA (2.2 mmol, 372 μL) was added and the mixture was microwaved at 100° C. for 2 h. Upon completion, the mixture was condensed and purified by flash column chromatography using a gradient of 0-2% MeOH/DCM to give tert-butyl 4-[[2-(2,6-dioxo-3-piperidyl)-1,3-dioxo-isoindolin-4-yl]amino]butanoate (209 mg, 49% yield). ESI-MS m/z: 593 ([M+H]⁺). ¹H-NMR (300 MHz, CD₂Cl₂): 8.47 (s, 1H), 7.51 (t, J=8.1 Hz, 1H), 7.07 (d, J=7.3 Hz, 1H), 6.96 (d, J=7.4 Hz, 1H), 6.49 (m, 1H), 5.11 (m, 1H), 4.91 (m, 1H), 3.71 (t, J=5.5 Hz, 2H), 3.58 (m, 14H), 3.48 (m, 2H), 3.25 (q, J=5.0 Hz, 2H), 2.78 (m, 3H), 2.12 (m, 1H), 1.41 (s, 9H).

4-[2-[2-[2-[2-(2-aminoethoxy) ethoxy]ethoxy]ethoxy]ethylamino]-2-(2,6-dioxo-3-piperidyl) isoindoline-1,3-dione (72): To a 10 mL round-bottle flask was added a solution of tert-butyl N-[2-[2-[2-[2-[2-[[2-(2,6-dioxo-3-piperidyl)-1,3-dioxo-isoindolin-4-yl]amino]ethoxy]ethoxy]ethoxy]ethoxy]ethyl]carbamate (0.35 mmol, 209 mg) in formic acid (2 mL). Then the mixture was stirred at room temperature for overnight. Upon completion, the mixture was condensed and purified by flash column chromatography using a gradient of 0-4% MeOH/DCM to give 4-[2-[2-[2-[2-(2-aminoethoxy) ethoxy]ethoxy]ethoxy]ethylamino]-2-(2,6-dioxo-3-piperidyl) isoindoline-1,3-dione (160 mg, 92% yield). ESI-MS m/z: 493 ([M+H]⁺).

N-[2-[2-[2-[2-[2-[[2-(2,6-dioxo-3-piperidyl)-1,3-dioxo-isoindolin-4-yl]amino]ethoxy]ethoxy]ethoxy]ethoxy]ethyl]-2-[4-[4-(4-isoquinolyl)phenyl]pyrazol-1-yl]acetamide (73): To a 10 mL round-bottle flask was added a solution of 2-[4-[4-(4-isoquinolyl)phenyl]pyrazol-1-yl]acetic acid (0.12 mmol, 40 mg) and 4-[2-[2-[2-[2-(2-aminoethoxy) ethoxy]ethoxy]ethoxy]ethylamino]-2-(2,6-dioxo-3-piperidyl) isoindoline-1,3-dione (0.12 mmol, 60 mg) in DMF (2 mL). Then EDC-HCl (0.24 mmol, 47 mg), HOAt (0.24 mmol, 33 mg), and N-methylmorpholine (0.61 mmol, 62 mg) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give N-[2-[2-[2-[2-[2-[[2-(2,6-dioxo-3-piperidyl)-1,3-dioxo-isoindolin-4-yl]amino]ethoxy]ethoxy]ethoxy]ethoxy]ethyl]-2-[4-[4-(4-isoquinolyl)phenyl]pyrazol-1-yl]acetamide (26 mg, 27% yield). ESI-MS m/z: 804 ([M+H]⁺). ¹H-NMR (300 MHz, CD₂Cl₂): 9.25 (s, 1H), 9.12 (s, 1H), 8.48 (s, 1H), 8.07 (d, J=8.0 Hz, 1H), 7.97 (q, J=8.5 Hz, 1H), 7.95 (s, 1H), 7.88 (s, 1H), 7.69 (m, 2H), 7.66 (d, J=8.0 Hz, 2H), 7.53 (d, J=8.0 Hz, 2H), 7.48 (t, J=8.0 Hz, 1H), 7.05 (d, J=7.0 Hz, 1H), 6.91 (d, J=7.0 Hz, 1H), 6.86 (m, 1H), 6.49 (m, 1H), 4.91 (m, 1H), 4.86 (s, 2H), 3.69 (t, J=5.0 Hz, 2H), 3.63-3.53 (m, 14H), 3.43 (q, J=4.8 Hz, 4H), 2.77 (m, 3H), 2.10 (m, 1H).

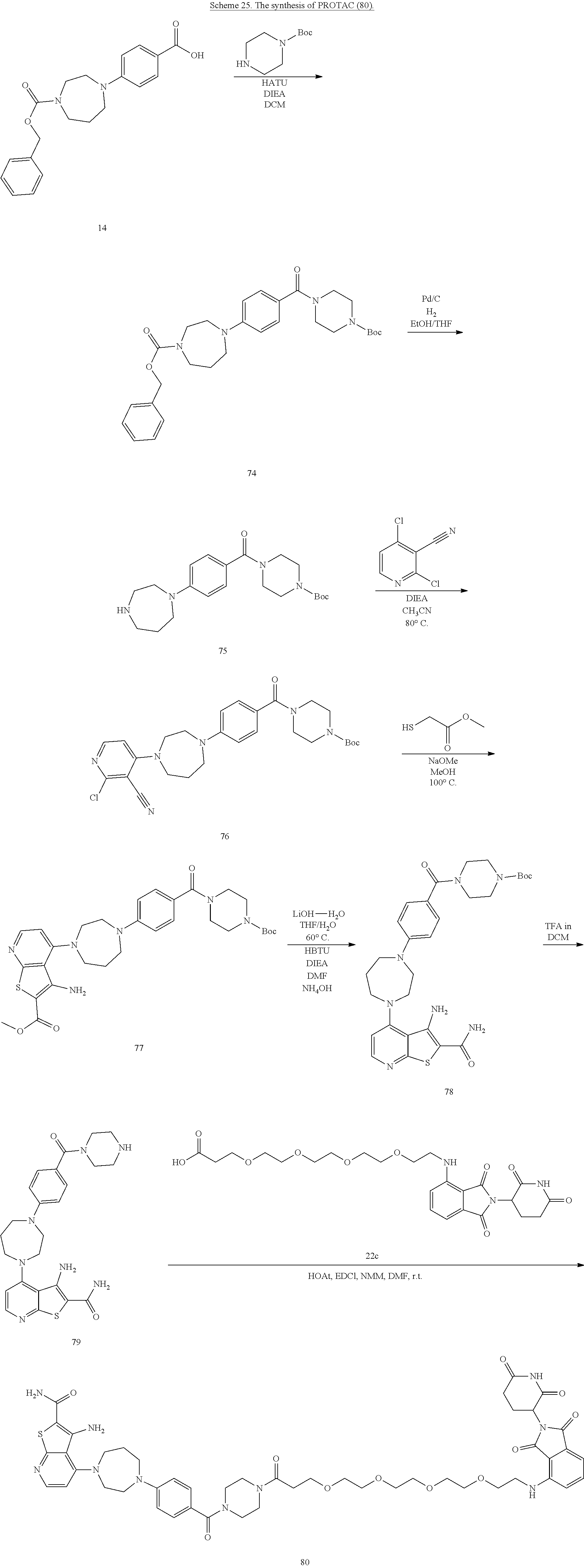

benzyl 4-(4-(4-(tert-butoxycarbonyl) piperazine-1-carbonyl)phenyl)-1,4-diazepane-1-carboxylate (74): To a 100 mL round-bottom flask was added a solution of 4-(4-((benzyloxy) carbonyl)-1,4-diazepan-1-yl) benzoic acid (0.49 mmol, 173 mg) in DMF (5 mL). Then HATU (0.73 mmol, 279 mg), DIEA (0.98 mmol, 126 mg) were added and the mixture was stirred at room temperature for 15 min. Then tert-butyl piperazine-1-carboxylate (0.49 mmol, 91 mg) was added and the mixture was stirred for another 2 h. Upon completion, water was added, extracted with DCM, the organic layers were combined, dried by Na₂SO₄, condensed, and purified flash column by using a gradient of 0-5% MeOH/DCM to give benzyl 4-(4-(4-(tert-butoxycarbonyl) piperazine-1-carbonyl)phenyl)-1,4-diazepane-1-carboxylate (125 mg, 49%). ESI-MS m/z: 523 ([M+H]⁺).

tert-butyl 4-[4-(1,4-diazepan-1-yl) benzoyl]piperazine-1-carboxylate (75): To a 25 ml round-bottom flask was added a solution of benzyl 4-[4-(4-tert-butoxycarbonylpiperazine-1-carbonyl)phenyl]-1,4-diazepane-1-carboxylate (1.2 mmol, 650 mg) in EtOH (4 mL) and ethyl acetate (4 mL). Then Pd/C (0.63 mmol, 67 mg) was added, the mixture was saturated with H₂, and stirred at room temperature for overnight. After that, the mixture was diluted with hexane, filtered, and the residue was washed with hexane, the filtration was condensed to give tert-butyl 4-[4-(1,4-diazepan-1-yl) benzoyl]piperazine-1-carboxylate (430 mg, 89%) and used without further purification ESI-MS m/z: 389 ([M+H]⁺) 1H-NMR (300 MHz, CDCl₃). 7.33 (d, J=8.8 Hz, 2H), 6.66 (d, J=8.8 Hz, 2H), 3.60 (m, 8H), 3.44 (m, 4H), 3.05 (m, 2H), 2.85 (m, 2H), 1.94 (m, 2H), 1.46 (s, 9H).

tert-butyl 4-[4-[4-(2-chloro-3-cyano-4-pyridyl)-1,4-diazepan-1-yl]benzoyl]piperazine-1-carboxylate (76). To a 100 mL round-bottom flask was added a solution of tert-butyl 4-[4-(1,4-diazepan-1-yl) benzoyl]piperazine-1-carboxylate (1.11 mmol, 430 mg) in acetonitrile (10 mL), then 2,4-dichloropyridine-3-carbonitrile (1.11 mmol, 191 mg) and DIPEA (2.21 mmol, 286 mg) were added. The mixture was stirred at 80° C. for overnight. Upon completion, the mixture was cooled to room temperature, condensed, diluted with water, extracted with DCM, the organic layers were combined, dried by Na₂SO₄, condensed, and purified by flash column chromatography using a gradient of 0-7% MeOH/DCM to give tert-butyl 4-[4-[4-(2-chloro-3-cyano-4-pyridyl)-1,4-diazepan-1-yl]benzoyl]piperazine-1-carboxylate (318 mg, 55%). ESI-MS m/z: 526 ([M+H]⁺).

methyl 3-amino-4-[4-[4-(4-tert-butoxycarbonylpiperazine-1-carbonyl)phenyl]-1,4-diazepan-1-yl]thieno[2,3-b]pyridine-2-carboxylate (77): To a 50 mL round-bottom flask was added a solution of tert-butyl 4-[4-[4-(2-chloro-3-cyano-4-pyridyl)-1,4-diazepan-1-yl]benzoyl]piperazine-1-carboxylate (0.57 mmol, 300 mg) in MeOH (5 mL), then methyl 2-sulfanylacetate (1.71 mmol, 182 mg) and MeONa (0.57 mmol, 31 mg) were added. The mixture was stirred at 100° C. for overnight. Upon completion, the mixture was condensed and purified by flash column chromatography using a gradient of 0-7% MeOH/DCM to give methyl 3-amino-4-[4-[4-(4-tert-butoxycarbonylpiperazine-1-carbonyl)phenyl]-1,4-diazepan-1-yl]thieno[2,3-b]pyridine-2-carboxylate (288 mg, 85%). ESI-MS m/z: 595 ([M+H]⁺). ¹H-NMR (300 MHz, CDCl₃): 8.48 (d, J=5.2 Hz, 1H), 7.37 (d, J=8.9 Hz, 2H), 6.91 (d, J=5.2 Hz, 1H), 6.77 (m, 2H), 6.72 (d, J=8.9 Hz, 2H), 3.86 (s, 3H), 3.80 (t, J=6.1 Hz, 2H), 3.64 (m, 6H), 3.47 (m, 4H), 3.70 (m, 2H), 3.27 (m, 2H), 2.20 (m, 2H), 1.47 (s, 9H).

tert-butyl 4-[4-[4-(3-amino-2-carbamoyl-thieno[2,3-b]pyridin-4-yl)-1,4-diazepan-1-yl]benzoyl]piperazine-1-carboxylate (78): To a 25 mL round-bottom flask was added a solution of methyl 3-amino-4-[4-[4-(4-tert-butoxycarbonylpiperazine-1-carbonyl)phenyl]-1,4-diazepan-1-yl]thieno[2,3-b]pyridine-2-carboxylate (0.21 mmol, 120 mg) in THF (3 mL) and water (3 mL), then LiOH—H₂O (0.42 mmol, 17 mg) was added. The mixture was stirred at 50° C. for overnight. Upon completion, the mixture was condensed and dissolved in DMF (5 mL). Then HATU (0.3 mmol, 114 mg), DIPEA (0.4 mmol, 52 mg) were added and stirred for 15 min. After that, NH₄OH (2.1 mmol, 74 mg) was added and the mixture was stirred for 2 h. Upon completion, the mixture was diluted with water, extracted with DCM, the organic layers were combined, dried by Na₂SO₄, condensed and purified by flash column chromatography using a gradient of 0-7% MeOH/DCM to give tert-butyl 4-[4-[4-(3-amino-2-carbamoyl-thieno[2,3-b]pyridin-4-yl)-1,4-diazepan-1-yl]benzoyl]piperazine-1-carboxylate (87 mg, 75%). ESI-MS m/z: 580 ([M+H]⁺). ¹H-NMR (300 MHz, CDCl₃): 8.47 (d, J=5.6 Hz, 1H), 7.37 (d, J=8.9 Hz, 2H), 6.98 (s, 2H), 6.93 (d, J=5.6 Hz, 1H), 6.72 (d, J=8.9 Hz, 2H), 5.39 (s, 2H), 3.79 (m, 2H), 3.64 (m, 6H), 3.46 (m, 4H), 3.37 (s, 2H), 3.27 (s, 2H), 2.21 (m, 2H), 1.47 (s, 9H).

3-amino-4-[4-[4-(piperazine-1-carbonyl)phenyl]-1,4-diazepan-1-yl]thieno[2,3-b]pyridine-2-carboxamide (79): To a 25 mL round-bottom flask was added a solution of tert-butyl 4-[4-[4-(3-amino-2-carbamoyl-thieno[2,3-b]pyridin-4-yl)-1,4-diazepan-1-yl]benzoyl]piperazine-1-carboxylate (0.11 mmol, 62 mg) in DCM (3 mL), then TFA (0.6 mL) was added. The mixture was stirred at room temperature for 4 h. Upon completion, the mixture was condensed to give 3-amino-4-[4-[4-(piperazine-1-carbonyl)phenyl]-1,4-diazepan-1-yl]thieno[2,3-b]pyridine-2-carboxamide (33 mg, 64%). ESI-MS m/z: 480 ([M+H]⁺). ¹H-NMR (300 MHz, CDCl₃): 8.47 (d, J=5.4 Hz, 1H), 7.37 (d, J=8.6 Hz, 2H), 6.99 (s, 2H), 6.93 (d, J=5.4 Hz, 1H), 6.72 (d, J=8.6 Hz, 2H), 5.40 (s, 2H), 3.79 (m, 2H), 3.65 (m, 6H), 3.38 (m, 2H), 3.27 (s, 2H), 2.91 (m, 4H), 2.21 (m, 2H), 1.98 (s, 1H).

3-amino-4-[4-[4-[4-[3-[2-[2-[2-[2-[[2-(2,6-dioxo-3-piperidyl)-1,3-dioxo-isoindolin-4-yl]amino]ethoxy]ethoxy]ethoxy]ethoxy]propanoyl]piperazine-1-carbonyl]phenyl]-1,4-diazepan-1-yl]thieno[2,3-b]pyridine-2-carboxamide (80): To a 10 mL round-bottle flask was added a solution of 3-amino-4-[4-[4-(piperazine-1-carbonyl)phenyl]-1,4-diazepan-1-yl]thieno[2,3-b]pyridine-2-carboxamide (0.083 mmol, 40 mg) and 3-[2-[2-[2-[2-[[2-(2,6-dioxo-3-piperidyl)-1,3-dioxo-isoindolin-4-yl]amino]ethoxy]ethoxy]ethoxy]ethoxy]propanoic acid (0.083 mmol, 44 mg) in DMF (2 mL). Then EDC-HCl (0.17 mmol, 32 mg), HOAt (0.17 mmol, 23 mg), and N-methylmorpholine (0.42 mmol, 42 mg) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give 3-amino-4-[4-[4-[4-[3-[2-[2-[2-[2-[[2-(2,6-dioxo-3-piperidyl)-1,3-dioxo-isoindolin-4-yl]amino]ethoxy]ethoxy]ethoxy]ethoxy]propanoyl]piperazine-1-carbonyl]phenyl]-1,4-diazepan-1-yl]thieno[2,3-b]pyridine-2-carboxamide (18 mg, 22% yield). ESI-MS m/z: 984 ([M+H]⁺). ¹H-NMR (300 MHz, CD₂Cl₂): 9.11 (s, 1H), 8.45 (d, J=5.1 Hz, 1H), 7.49 (t, J=7.4 Hz, 1H), 7.35 (m, 3H), 7.06 (d, J=7.4 Hz, 1H), 6.96 (m, 2H), 6.87 (s, 1H), 6.75 (d, J=9.6 Hz, 2H), 6.49 (m, 1H), 5.65 (s, 2H), 4.91 (m, 1H), 3.80 (m, 2H), 3.76 (t, J=6.5 Hz, 2H), 3.70 (t, J=6.5 Hz, 2H), 3.64-3.54 (m, 18H), 3.46 (q, J=5.4 Hz, 2H), 3.38 (m, 2H), 3.29 (m, 2H), 3.05 (m, 2H), 2.96 (m, 2H), 2.79 (m, 3H), 2.67 (t, J=7.2 Hz, 2H), 2.15 (m, 1H), 1.86 (m, 2H).

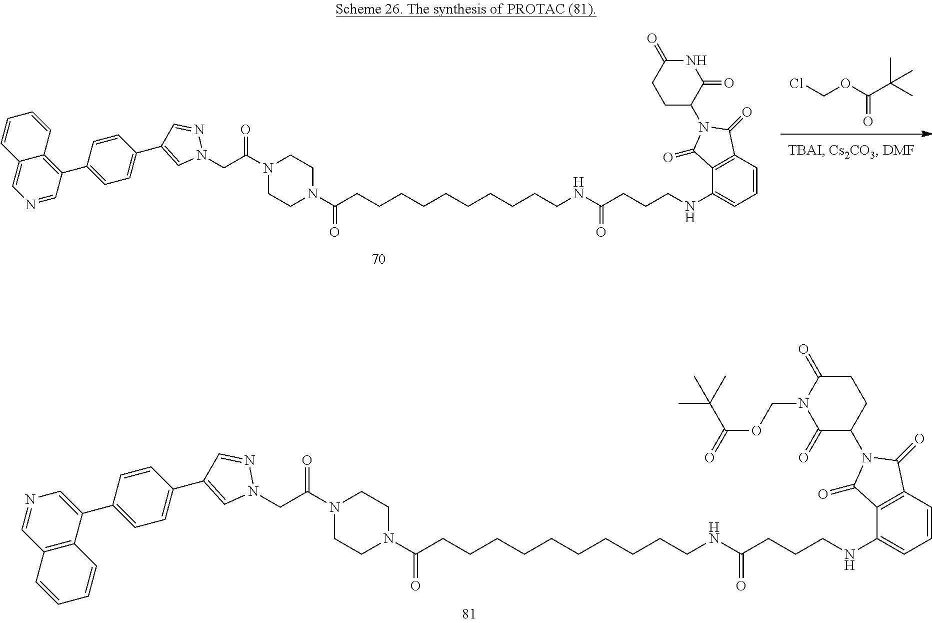

(3-(4-((4-((11-(4-(2-(4-(4-(isoquinolin-4-yl)phenyl)-1H-pyrazol-1-yl) acetyl) piperazin-1-yl)-11-oxoundecyl)amino)-4-oxobutyl)amino)-1,3-dioxoisoindolin-2-yl)-2,6-dioxopiperidin-1-yl)methyl pivalate (81): To a 10 mL round-bottle flask was added a solution of 4-((2-(2,6-dioxopiperidin-3-yl)-1,3-dioxoisoindolin-4-yl)amino)-N-(11-(4-(2-(4-(4-(isoquinolin-4-yl)phenyl)-1H-pyrazol-1-yl) acetyl) piperazin-1-yl)-11-oxoundecyl) butanamide (0.054 mmol, 50 mg) in DMF (2 mL), then chloromethyl pivalate (0.081 mmol, 12 mg), cesium carbonate (0.108 mmol, 35 mg), TBAI (cat.) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give 3-amino-4-[4-[4-[4-[3-[2-[2-[2-[2-[[2-(2,6-dioxo-3-piperidyl)-1,3-dioxo-isoindolin-4-yl]amino]ethoxy]ethoxy]ethoxy]ethoxy]propanoyl]piperazine-1-carbonyl]phenyl]-1,4-diazepan-1-yl]thieno[2,3-b]pyridine-2-carboxamide (26 mg, 47% yield). ESI-MS m/z: 1036 ([M+H]⁺). ¹H-NMR (300 MHz, CD₂Cl₂): 9.24 (s, 1H), 8.48 (s, 1H), 8.07 (d, J=7.9 Hz, 1H), 7.97 (d, J=7.9 Hz, 1H), 7.88 (s, 2H), 7.68 (m, 4H), 7.51 (m, 3H), 7.05 (d, J=7.3 Hz, 1H), 6.98 (d, J=8.7 Hz, 1H), 6.32 (t, J=6.0 Hz, 1H), 5.80 (s, 2H), 5.63 (m, 1H), 5.07 (s, 2H), 4.97 (m, 1H), 3.67-3.52 (m, 6H), 3.48 (m, 2H), 3.33 (q, J=7.2 Hz, 2H), 3.19 (q, J=6.5 Hz, 2H), 2.86 (m, 3H), 2.31 (t, J=7.9 Hz, 2H), 2.25 (t, J=7.9 Hz, 2H), 2.10 (m, 1H), 1.95 (m, 2H), 1.58 (m, 2H), 1.45 (m, 2H), 1.28 (s, 12H), 1.16 (S, 9H).

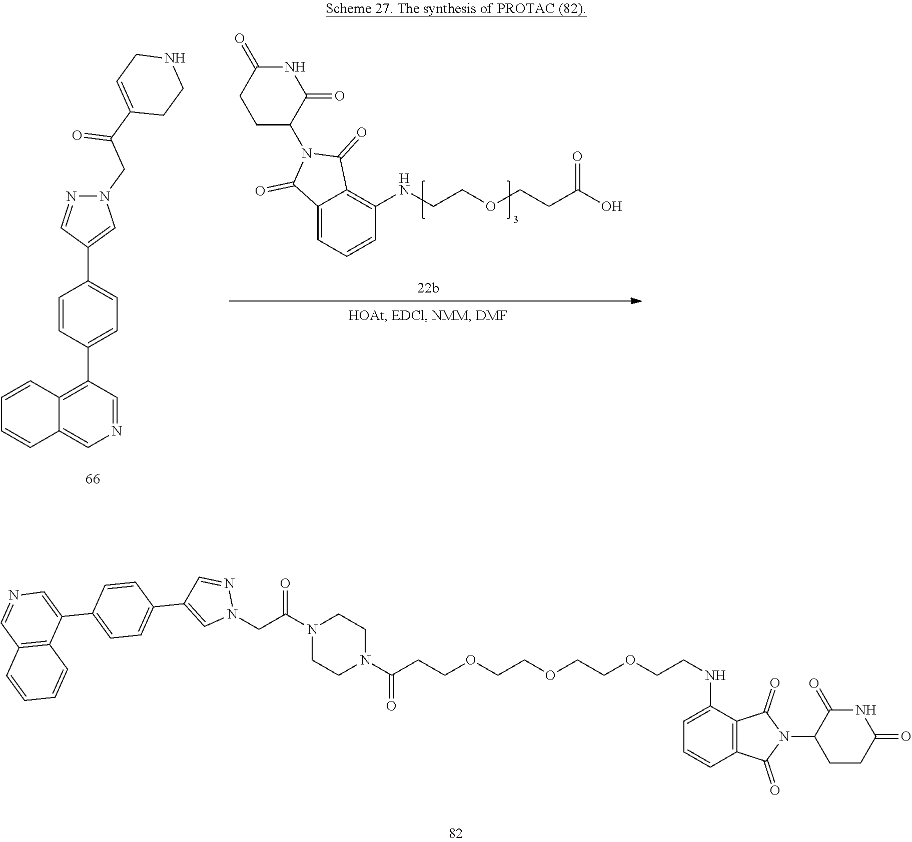

2-(2,6-dioxopiperidin-3-yl)-4-((2-(2-(2-(3-(4-(2-(4-(4-(isoquinolin-4-yl)phenyl)-1H-pyrazol-1-yl) acetyl) piperazin-1-yl)-3-oxopropoxy) ethoxy) ethoxy)ethyl)amino) isoindoline-1,3-dione (82): To a 10 mL round-bottle flask was added a solution of 2-(4-(4-(isoquinolin-4-yl)phenyl)-1H-pyrazol-1-yl)-1-(piperazin-1-yl)ethan-1-one (0.10 mmol, 40 mg) and 3-(2-(2-(2-((2-(2,6-dioxopiperidin-3-yl)-1,3-dioxoisoindolin-4-yl)amino) ethoxy) ethoxy) ethoxy) propanoic acid (0.083 mmol, 48 mg) in DMF (2 mL). Then EDC-HCl (0.20 mmol, 38 mg), HOAt (0.20 mmol, 44 mg), and N-methylmorpholine (0.50 mmol, 50 mg) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give 2-(2,6-dioxopiperidin-3-yl)-4-((2-(2-(2-(3-(4-(2-(4-(4-(isoquinolin-4-yl)phenyl)-1H-pyrazol-1-yl) acetyl) piperazin-1-yl)-3-oxopropoxy) ethoxy) ethoxy)ethyl)amino) isoindoline-1,3-dione (21 mg, 25% yield). ESI-MS m/z: 857 ([M+H]⁺). ¹H-NMR (300 MHz, CD₂Cl₂): 9.24 (s, 1H), 9.09 (s, 1H), 8.48 (s, 1H), 8.06 (d, J=8.2 Hz, 1H), 7.97 (d, J=8.2 Hz, 1H), 7.89 (s, 2H), 7.67 (m, 4H), 7.51 (m, 3H), 7.07 (d, J=7.2 Hz, 1H), 6.95 (d, J=8.4 Hz, 1H), 6.50 (t, J=5.0 Hz, 1H), 5.08 (s, 2H), 4.92 (m, 1H), 3.76-3.46 (m, 22H), 2.77 (m, 3H), 2.61 (m, 2H), 2.11 (m, 1H).

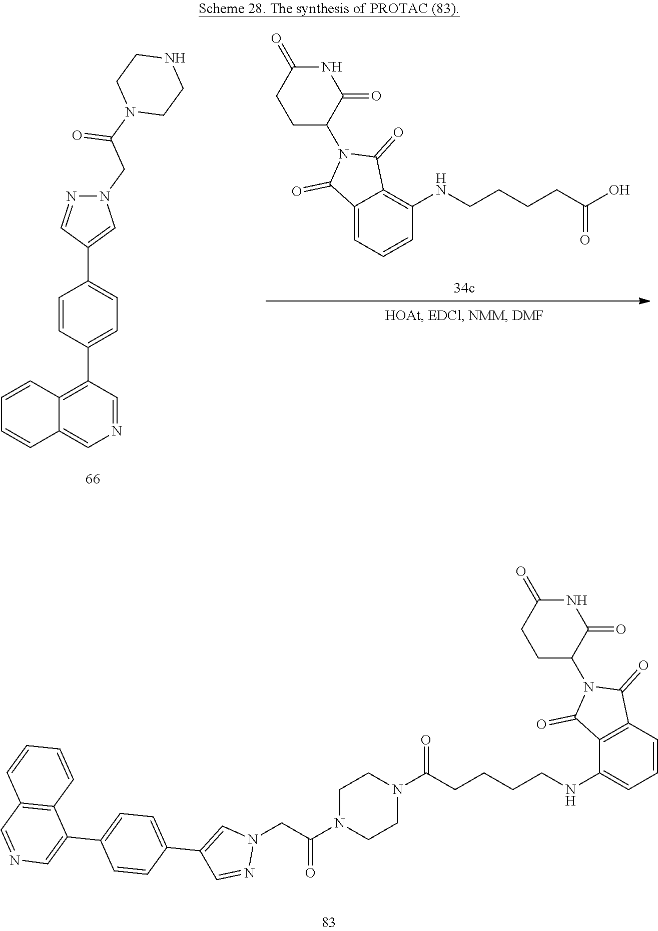 [SMILES: O=C(Cn1cc(-c2ccc(-c3cncc4ccccc34)cc2)cn1)N1CCNCC1 ; O=C(O)CCCCNc1cccc2c1C(=O)N(C1CCC(=O)NC1=O)C2=O ; O=C1CCC(N2C(=O)c3cccc(NCCCCC(=O)N4CCN(C(=O)Cn5cc(-c6ccc(-c7cncc8ccccc78)cc6)cn5)CC4)c3C2=O)C(=O)N1]

2-(2,6-dioxopiperidin-3-yl)-4-((5-(4-(2-(4-(4-(isoquinolin-4-yl)phenyl)-1H-pyrazol-1-yl) acetyl) piperazin-1-yl)-5-oxopentyl)amino) isoindoline-1,3-dione (83): To a 10 mL round-bottle flask was added a solution of 2-(4-(4-(isoquinolin-4-yl)phenyl)-1H-pyrazol-1-yl)-1-(piperazin-1-yl)ethan-1-one (0.10 mmol, 40 mg) and 5-((2-(2,6-dioxopiperidin-3-yl)-1,3-dioxoisoindolin-4-yl)amino) pentanoic acid (0.10 mmol, 37 mg) in DMF (2 mL). Then EDC-HCl (0.20 mmol, 38 mg), HOAt (0.20 mmol, 44 mg), and N-methylmorpholine (0.50 mmol, 50 mg) were added and the mixture was stirred at room temperature for overnight. Upon completion, the mixture was diluted with DCM, added with water, extracted with DCM, the organic layers were combined, dried by Na₂SO₄, condensed and purified by flash column chromatography using a gradient of 0-10% MeOH/DCM to give 2-(2,6-dioxopiperidin-3-yl)-4-((5-(4-(2-(4-(4-(isoquinolin-4-yl)phenyl)-1H-pyrazol-1-yl) acetyl) piperazin-1-yl)-5-oxopentyl)amino) isoindoline-1,3-dione (26 mg, 35% yield). ESI-MS m/z: 753 ([M+H]⁺). ¹H-NMR (300 MHz, CD₂Cl₂): 10.06 (s, 1H), 9.25 (s, 1H), 8.48 (s, 1H), 8.07 (d, J=8.3 Hz, 1H), 7.97 (d, J=8.3 Hz, 1H), 7.95 (d, J=6.4 Hz, 1H), 7.90 (d, J=6.4 Hz, 1H), 7.68 (m, 4H), 7.53 (d, J=8.0 Hz, 2H), 7.50 (m, 1H), 7.07 (m, 1H), 6.93 (m, 1H), 6.29 (t, J=5.3 Hz, 1H), 5.10 (s, 2H), 4.92 (m, 1H), 3.63-3.34 (m, 10H), 2.79 (m, 3H), 2.39 (m, 2H), 2.11 (m, 1H), 1.74 (s, 4H).

### REFERENCES

Adler, A. S., McCleland, M. L., Truong, T., Lau, S., Modrusan, Z., Soukup, T. M., Roose-Girma, M., Blackwood, E. M., and Firestein, R. (2012). CDK8 maintains tumor dedifferentiation and embryonic stem cell pluripotency. Cancer Res 72, 2129-2139. 0008-5472.CAN-11-3886 [pii]; 10.1158/0008-5472.CAN-11-3886 [doi].
Al-Sanea, M. M. (2020). Synthesis and biological evaluation of small molecule modulators of CDK8/Cyclin C complex with phenylaminoquinoline scaffold. PeerJ 8, e8649. 10.7717/peerj.8649.
Amirhosseini, M., Bernhardsson, M., Lang, P., Andersson, G., Flygare, J., and Fahlgren, A. (2019). Cyclin-dependent kinase 8/19 inhibition suppresses osteoclastogenesis by downregulating RANK and promotes osteoblast mineralization and cancellous bone healing. Journal of cellular physiology. 10.1002/jcp.28321.
Audetat, K. A., Galbraith, M. D., Odell, A. T., Lee, T., Pandey, A., Espinosa, J. M., Dowell, R. D., and Taatjes, D. J. (2017). A Kinase-Independent Role for Cyclin-Dependent Kinase 19 in p53 Response. Mol Cell Biol 37. 10.1128/MCB.00626-16.
Barankiewicz, J., Salomon-Perzyński, A., Misiewicz-Krzemińska, I., and Lech-Maranda, E. (2022). CRL4 (CRBN) E3 Ligase Complex as a Therapeutic Target in Multiple Myeloma. Cancers 14. 10.3390/cancers 14184492.
Barette, C., Jariel-Encontre, I., Piechaczyk, M., and Piette, J. (2001). Human cyclin C protein is stabilized by its associated kinase cdk8, independently of its catalytic activity. Oncogene 20, 551-562. 10.1038/sj.onc. 1204129 [doi].
Butler, M., Chotiwan, N., Brewster, C. D., DiLisio, J. E., Ackart, D. F., Podell, B. K., Basaraba, R. J., Perera, R., Quackenbush, S. L., and Rovnak, J. (2020). Cyclin-Dependent Kinases 8 and 19 Regulate Host Cell Metabolism during Dengue Virus Serotype 2 Infection. Viruses 12. 10.3390/v12060654.
Chen, M., Li, J., Liang, J., Thompson, Z. S., Kathrein, K., Broude, E. V., and Roninson, I. B. (2019). Systemic Toxicity Reported for CDK8/19 Inhibitors CCT251921 and MSC2530818 Is Not Due to Target Inhibition. Cells 8. 10.3390/cells8111413.
Clarke, P. A., Ortiz-Ruiz, M. J., TePoele, R., Adeniji-Popoola, O., Box, G., Court, W., Czasch, S., El Bawab, S., Esdar, C., Ewan, K., et al. (2016). Assessing the mechanism and therapeutic potential of modulators of the human Mediator complex-associated protein kinases. Elife 5. 10.7554/eLife.20722.
Grandjean, J. M., Jiu, A. Y., West, J. W., Aoyagi, A., Droege, D. G., Elepano, M., Hirasawa, M., Hirouchi, M., Murakami, R., Lee, J, et al. (2020) Discovery of 4-Piperazine Isoquinoline Derivatives as Potent and Brain-Permeable Tau Prion Inhibitors with CDK8 Activity. ACS Med Chem Lett 11, 127-132. 10.1021/acsmedchemlett.9b00480.
Hatcher, J. M., Wang, E. S., Johannessen, L., Kwiatkowski, N., Sim, T., and Gray, N. S. (2018). Development of Highly Potent and Selective Steroidal Inhibitors and Degraders of CDK8. ACS Med Chem Lett 9, 540-545. 10.1021/acsmedchemlett.8b00011.
Hofmann, M. H., Mani, R., Engelhardt, H., Impagnatiello, M. A., Carotta, S., Kerenyi, M., Lorenzo-Herrero, S., Böttcher, J., Scharn, D., Arnhof, H., et al. (2020). Selective and Potent CDK8/19 Inhibitors Enhance NK-Cell Activity and Promote Tumor Surveillance. Mol Cancer Ther 19, 1018-1030. 10.1158/1535-7163.Mct-19-0789.
Johannessen, L., Sundberg, T. B., O'Connell, D. J., Kolde, R., Berstler, J., Billings, K. J., Khor, B., Seashore-Ludlow, B., Fassl, A., Russell, C. N., et al. (2017). Small-molecule studies identify CDK8 as a regulator of IL-10 in myeloid cells. Nat Chem Biol 13, 1102-+. 10.1038/Nchembio.2458.
Kapoor, A., Goldberg, M. S., Cumberland, L. K., Ratnakumar, K., Segura, M. F., Emanuel, P. O., Menendez, S., Vardabasso, C., Leroy, G., Vidal, C. I., et al. (2010). The histone variant macroH2A suppresses melanoma progression through regulation of CDK8. Nature 468, 1105-1109. nature09590 [pii]; 10.1038/nature09590 [doi].
Li, Q., Feng, K., Liu, J., and Ren, Y. (2020). Molecular modeling studies of novel naphthyridine and isoquinoline derivatives as CDK8 inhibitors. J Biomol Struct Dyn, 1-15. 10.1080/07391102.2020.1797537.
Liang, J., Chen, M., Hughes, D., Chumanevich, A. A., Altilia, S., Kaza, V., Lim, C. U., Kiaris, H., Mythreye, K., Pena, M. M., et al. (2018). CDK8 Selectively Promotes the Growth of Colon Cancer Metastases in the Liver by Regulating Gene Expression of TIMP3 and Matrix Metalloproteinases. Cancer Res 78, 6594-6606. 10.1158/0008-5472.Can-18-1583.
Lynch, C. J., Bernad, R., Martínez-Val, A., Shahbazi, M. N., Nóbrega-Pereira, S., Calvo, I., Blanco-Aparicio, C., Tarantino, C., Garreta, E., Richart-Ginés, L., et al. (2020). Global hyperactivation of enhancers stabilizes human and mouse naive pluripotency through inhibition of CDK8/19 Mediator kinases. Nat Cell Biol 22, 1223-1238. 10.1038/s41556-020-0573-1.
Ma, D., Chen, X., Shen, X. B., Sheng, L. Q., and Liu, X. H. (2020). Binding patterns and structure-activity relationship of CDK8 inhibitors. Bioorg Chem 96, 103624. 10.1016/j.bioorg.2020.103624.
Martínez-González, S., Garcia, A. B., Albarrán, M. I., Cebriá, A., Amezquita-Alves, A., García-Campos, F. J., Martinez-Gago, J., Martínez-Torrecuadrada, J., Muñoz, I., Blanco-Aparicio, C., and Pastor, J. (2020). Pyrido[2,3-b][1,5]benzoxazepin-5 (6H)-one derivatives as CDK8 inhibitors. Eur J Med Chem 201, 112443. 10.1016/j.ejmech.2020.112443.
McDermott, M. S., Chumanevich, A. A., Lim, C. U., Liang, J., Chen, M., Altilia, S., Oliver, D., Rae, J. M., Shtutman, M., Kiaris, H., et al. (2017). Inhibition of CDK8 mediator kinase suppresses estrogen dependent transcription and the growth of estrogen receptor positive breast cancer. Oncotarget 8, 12558-12575. 10.18632/oncotarget. 14894.
Menzl, I., Zhang, T., Berger-Becvar, A., Grausenburger, R., Heller, G., Prchal-Murphy, M., Edlinger, L., Knab, V. M., Uras, I. Z., Grundschober, E., et al. (2019). A kinase-independent role for CDK8 in BCR-ABL1(+) leukemia. Nat Commun 10, 4741. 10.1038/s41467-019-12656-x.
Pelish, H. E., Liau, B. B., Nitulescu, I. I., Tangpeerachaikul, A., Poss, Z. C., Da Silva, D. H., Caruso, B. T., Arefolov, A., Fadeyi, O., Christie, A. L., et al. (2015). Mediator kinase inhibition further activates super-enhancer-associated genes in AML. Nature 526, 273-276. nature14904 [pii]; 10.1038/nature14904 [doi].
Philip, S., Kumarasiri, M., Teo, T., Yu, M., and Wang, S. (2018). Cyclin-Dependent Kinase 8: A New Hope in Targeted Cancer Therapy? J Med Chem. 10.1021/acs.jmedchem. 7b00901.
Porter, D. C., Farmaki, E., Altilia, S., Schools, G. P., West, D. K., Chen, M., Chang, B. D., Puzyrev, A. T., Lim, C. U., Rokow-Kittell, R., et al. (2012). Cyclin-dependent kinase 8 mediates chemotherapy-induced tumor-promoting paracrine activities. Proc. Natl. Acad. Sci. U.S.A 109, 13799-13804. 1206906109 [pii]; 10.1073/pnas. 1206906109 [doi].
Roninson, I. B., Györffy, B., Mack, Z. T., Shtil, A. A., Shtutman, M. S., Chen, M., and Broude, E. V. (2019). Identifying Cancers Impacted by CDK8/19. Cells 8. 10.3390/cells8080821.
Samarasinghe, K. T. G., and Crews, C. M. (2021). Targeted protein degradation: a promise for undruggable proteins. Cell Chem Biol. 10.1016/j.chembiol.2021.04.011.
Solum, E., Hansen, T. V., Aesoy, R., and Herfindal, L. (2020). New CDK8 inhibitors as potential anti-leukemic agents-Design, synthesis and biological evaluation. Bioorganic & medicinal chemistry 28, 115461. 10.1016/j.bmc.2020.115461.
Steinparzer, I., Sedlyarov, V., Rubin, J. D., Eislmayr, K., Galbraith, M. D., Levandowski, C. B., Vcelkova, T., Sneezum, L., Wascher, F., Amman, F., et al. (2019). Transcriptional Responses to IFN-γ Require Mediator Kinase-Dependent Pause Release and Mechanistically Distinct CDK8 and CDK19 Functions. Mol Cell 76, 485-499.e488. 10.1016/j.molcel.2019.07.034.
Walz, A., Ugolkov, A., Chandra, S., Kozikowski, A., Carneiro, B. A., O'Halloran, T. V., Giles, F. J., Billadeau, D. D., and Mazar, A. P. (2017). Molecular Pathways: Revisiting Glycogen Synthase Kinase-3beta as a Target for the Treatment of Cancer. Clin Cancer Res 23, 1891-1897. 10.1158/1078-0432.Ccr-15-2240.
Wei, M., Zhao, R., Cao, Y., Wei, Y., Li, M., Dong, Z., Liu, Y., Ruan, H., Li, Y., Cao, S., et al. (2021). First orally bioavailable prodrug of proteolysis targeting chimera (PROTAC) degrades cyclin-dependent kinases 2/4/6 in vivo. Eur J Med Chem 209, 112903. 10.1016/j.ejmech.2020.112903.
Westerling, T., Kuuluvainen, E., and Makela, T. P. (2007). Cdk8 is essential for preimplantation mouse development. Mol. Cell Biol 27, 6177-6182.
Xi, M., Chen, T., Wu, C., Gao, X., Wu, Y., Luo, X., Du, K., Yu, L., Cai, T., Shen, R., and Sun, H. (2019). CDK8 as a therapeutic target for cancers and recent developments in discovery of CDK8 inhibitors. Eur J Med Chem 164, 77-91. 10.1016/j.ejmech.2018.11.076.
Yu, M., Long, Y., Yang, Y., Li, M., Teo, T., Noll, B., Philip, S., and Wang, S. (2021a). Discovery of a potent, highly selective, and orally bioavailable inhibitor of CDK8 through a structure-based optimisation. Eur J Med Chem 218, 113391. 10.1016/j.ejmech.2021.113391.
Yu, M., Teo, T., Yang, Y., Li, M., Long, Y., Philip, S., Noll, B., Heinemann, G. K., Diab, S., Eldi, P., et al. (2021b). Potent and orally bioavailable CDK8 inhibitors: Design, synthesis, structure-activity relationship analysis and biological evaluation. Eur J Med Chem 214, 113248. 10.1016/j.ejmech.2021.113248.

1. A heterobifunctional compound, a prodrug thereof, or a pharmaceutically acceptable salt thereof, comprising a CDK8/19 targeting moiety, an E3 ubiquitin ligase ligand moiety, and a linker linking the CDK8/19 targeting moiety and the ubiquitin ligase ligand moiety, wherein the CDK8/19 targeting moiety comprises 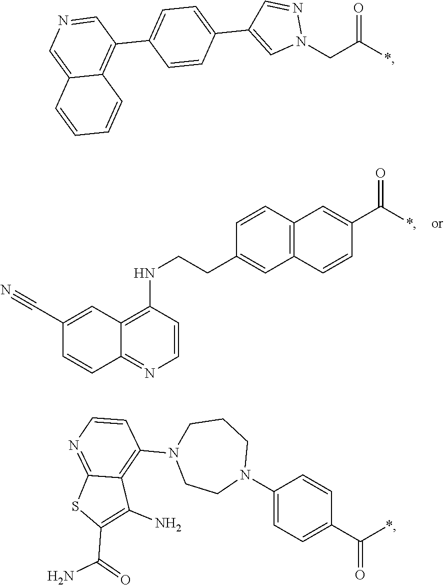
wherein * indicates a point of attachment of the CDK8/19 targeting moiety for the compound.

2. The compound of claim 1, or the pharmaceutically acceptable salt thereof, wherein the compound is IA7882, IA7843, IA7861, IA7886, HP8580, HV5957, IA7858, HV5958, IA7813, HP8579, IA7812, IA7848, HP8581, IA7868, IA7892, IA7869, IA7835, IA7840, IA7814, IA7822, IA7830, IA7819, IA7862, HP8553, HP8575, IA7859, HP8578, IA7860, IA7807, IA7803, IA7875, IA7891, IA7893, IA7894, or IA7895.

3. The compound of claim 1, wherein the CDK8/19 targeting moiety comprises

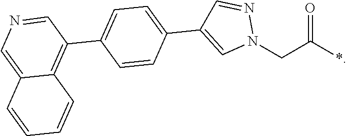

4. The compound of claim 3, wherein the compound is IA7893, IA7882, IA7886, or IA7893.

5. The compound of claim 3, wherein the CDK8/19 ligand comprises a piperazine between the CDK8/19 targeting moiety and the linker and/or wherein the ubiquitin ligase ligand moiety comprises a pomalidomide moiety or VH032 moiety.

6.-9. (canceled)

10. The compound of claim 1, wherein the CDK8/19 targeting moiety comprises

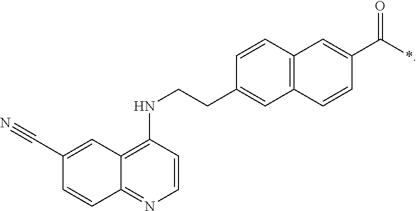

11. The compound of claim 10, wherein the compound is HP8580 or IA7843.

12. The compound of claim 10, wherein the CDK8/19 ligand comprises a piperazine between the CDK8/19 targeting moiety and the linker and/or wherein the ubiquitin ligase ligand moiety comprises a pomalidomide moiety or VH032 moiety.

13.-16. (canceled)

17. The compound of claim 1, wherein the CDK8/19 targeting moiety comprises

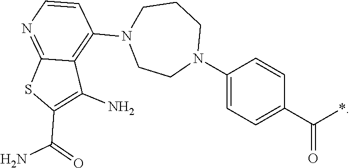

18. The compound of claim 17, wherein the compound is IA7892.

19. The compound of claim 17, wherein the CDK8/19 ligand comprises a piperazine between the CDK8/19 targeting moiety and the linker and/or wherein the ubiquitin ligase ligand moiety comprises a pomalidomide moiety or VH032 moiety.

20. (canceled)

21. (canceled)

22. A prodrug of the compound of claim 1, or a pharmaceutically acceptable salt thereof, wherein the prodrug comprises a methyl pivalate group.

23. (canceled)

24. A pharmaceutical composition comprising the compound of claim 1, a prodrug thereof, or a pharmaceutically acceptable salt thereof, and a pharmaceutically acceptable excipient, carrier, or diluent.

25. The pharmaceutical composition of claim 24, wherein the pharmaceutical composition comprises the prodrug and the prodrug comprises a methyl pivalate group.

26. A method for treating a subject, the method comprising administering to the subject the compound of claim 1, a prodrug thereof, or a pharmaceutically acceptable salt thereof.

27. The method of claim 26, wherein the subject is in need of a treatment for a cancer and/or an agent to reduce drug resistance to an anticancer agent.

28. The method of claim 27, wherein the cancer is a blood, prostate, breast, colon, nervous system, or osteo cancer.

29. The method of claim 27, wherein the cancer is:
(a) a leukemia;
(b) multiple myeloma;
(c) CDK8 and/or CDK19 dependent; or
(d) Cyclin C (CCNC) dependent.

30.-33. (canceled)

34. The method of claim 26, wherein the subject has a viral disease, an inflammation-associated disease, a ribosomopathy, a condition characterized by a reduced number of hematopoietic stem cells and/or progenitor cells, or a bone anabolic disorder.

35.-38. (canceled)

39. The method of claim 26, wherein the method comprises administering the prodrug and wherein the prodrug comprises a methyl pivalate group.

40.-53. (canceled)
